(19) United States
(12) Patent Application Publication
(10) Pub. No.: US 2026/0209728 A1
(43) Pub. Date: Jul. 23, 2026

(54) CARBOHYDRATE ESTERASE FAMILY 1 (CE1) POLYPEPTIDES HAVING FERULIC ACID ESTERASE AND/OR ACETYL XYLAN ESTERASE ACTIVITY AND POLYNUCLEOTIDES ENCODING SAME

(71) Applicant: Novozymes A/S, Bagsvaerd (DK)

(72) Inventors: Chee-Leong SOONG, Raleigh, NC (US); Ye LIU, Beijing (CN); Xiangyu CAI, Beijing (CN); Hong Zhi HUANG, Beijing (CN)

(73) Assignee: Novozymes A/S, Bagsvaerd (DK)

(21) Appl. No.: 19/138,069

(22) PCT Filed: Dec. 11, 2023
(86) PCT No.: PCT/US2023/083340

§ 371 Date: Jun. 11, 2025

(30) Foreign Application Priority Data
Dec. 19, 2022 (WO) PCT/CN2022/140028
Jun. 16, 2023 (WO) PCT/CN2023/100824

(51) Int. Cl. C12N 9/18; C12N 9/24; C12P 19/14

(52) U.S. Cl. CPC C12N 9/18; C12N 9/2402; C12P 19/14; C12Y 301/01072; C12Y 301/01073; C12Y 302/01037; C12Y 302/01055; C12Y 302/0108

(57) ABSTRACT

The present invention relates to carbohydrate esterase family 1 (CE1) polypeptides having ferulic acid esterase and/or acetyl xylan esterase activity and polynucleotides encoding the polypeptides. The invention also relates to nucleic acid constructs, vectors, and host cells comprising the polynucleotides as well as methods of producing and using the polypeptides. The invention also relates to compositions comprising a CE1 family polypeptide having ferulic acid esterase and/or acetyl xylan esterase activity and use of the compositions for solubilizing hemicellulosic fiber.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to carbohydrate esterase family 1 (CE1) polypeptides having ferulic acid esterase and/or acetyl xylan esterase activity, polynucleotides encoding the polypeptides, nucleic acid constructs, vectors, and host cells comprising the polynucleotides as well as methods of producing and using the polypeptides. The present invention also relates to compositions comprising the CE1 family polypeptides and use of the compositions for solubilizing hemicellulosic fiber.

### Description of the Related Art

Conversion of cellulosic feedstocks into biofuels is challenging due to their high recalcitrance, typically involved combination of thermochemical pretreatment followed by adding cellulase and hemicellulase enzymes to release soluble carbohydrates. With government sustainability initiatives, the biofuels industry is incentivized to produce ethanol from corn fiber at existing corn ethanol facilities. Corn fiber comprises 10% of the weight of corn kernels and consists of cellulose and hemicellulose from the aleurone and pericarp layers. In ethanol facilities, corn fiber ends up in the Distillers Dried Grains with Solubles (DDGS). Enzymatic hydrolysis of the hemicellulose portion of the corn fiber to monomeric C5 sugars such as xylose and arabinose simultaneously with fermentation of the C5 sugars to ethanol by C5 fermenting yeast, and leveraging existing infrastructure, would allow ethanol plants to produce additional cellulosic ethanol yield from the same amount of corn. Additional benefits from corn fiber degradation include better DDGS feed quality from enriched protein content for animal feed and the lower fiber content of DDGS would potentially qualify for access to the monogastric and aquaculture animal feed market.

The arabinoxylan backbone in corn fiber is composed of a xylan backbone of β-(1,4)-linked D-xylopyranosyl residues that highly substituted with arabinose side chains and to a lesser extent with glucuronic acid residues. The main substitutions of arabinose residues linked to the O-2 or O-3 position on monosubstituted xylopyranosyls or to both O-2 and O-3 on doubly substituted xylopyranosyl units. In addition to arabinose, the xylan backbone can be substituted with D-galactopyranosyl and D-glucuronyl residues, and/or with acetyl groups. Acetic acid is esterified directly to the xylan backbone in position O-2 or O-3, whereas hydroxycinnamic acids such as ferulic acid, p-coumaric acid, and dehydrodimers of ferulic acid are esterified to arabinofuranosyls in position O-5. It has also been reported that xylan is further substituted with xylopyranosyls by a (1-3)-linkage and that the arabinofuranosyls can be further decorated with xylopyranosyls or even L-galactopyranosyls. Because of the highly branched substitution by different moieties, enzymatic degradation of corn fiber arabinoxylan to monomeric C5 sugars requires concerted action of a mixture of debranching and depolymerizing activities. Debranching activities mainly include α-L-arabinofuranosidases (EC 3.2.1.55) (α-AraFs), feruloyl esterases (EC 3.1.1.73), α-glucuronidases (EC 3.2.1.139), and/or acetyl xylan esterases (EC 3.1.1.72), while depolymerization relies on endo-1,4-β-xylanase (EC 3.2.1.8) and β-xylosidase (EC 3.2.1.37) (BX) activities.

WO 2006/114095 “D1” describes a process and composition for hydrolyzing arabinoxylan, which includes contacting an arabinoxylan containing substrate with an enzyme having activity toward di-substituted arabinoses, e.g., such as a Glycoside Hydrolyase Family 43 (GH43) alpha-L-arabinofuranosidase, and an enzyme having activity towards C2- or C3-position mono-substituted arabinoses, e.g., such as a GH Family 51, 54 or 62 alpha-L-arabinofuranosidase. D1 teaches that when the two arabinofuranosidases are added to an arabinoxylan solution the resulting products will be high molecular weight linear xylose polymers and arabinose molecules that allow for an easy separation of the linear xylose polymer by known techniques from arabinose, which may be further partially digested with enzyme activities, such as beta-xylosidase (preferably GH3), and/or endo-1, 4-beta-xylanase (preferably GH10 or GH11), to yield xylo-oligosaccharides. D1 further teaches that when both endo-1,4-beta-xylanase and a beta-xylosidase are added to purified linear xylose polymers the resulting product will be xylose that is essentially free of arabinose substituents, and that for degradation of even more complex substrates, or where a more complete degradation is required, the presence of even further enzyme activities may be desired, such as acetyl xylan esterase (EC 3.1.1.72) and/or feruloyl esterase (EC 3.1.1.73) and/or alpha-glucuronidase (EC. 3.2.1.139).

However, supply chain disruptions and inflation have driven up the cost of raw material inputs for producing the enzymes needed for completely hydrolyzing complex arabinoxylan substrates, diminishing financial incentives for ethanol facilities to purchase additional enzymes for producing cellulosic ethanol from corn. Because conventional wisdom suggests all seven enzymatic activities are required to maximize cellulosic ethanol yields from corn, there exists a need for improved processes, and compositions capable of increasing cellulosic ethanol yields by releasing more monomeric arabinose and xylose with less enzymatic activities, and at a lower cost that is more profitable for corn ethanol facilities to maximize cellulosic ethanol yields from their existing corn inputs.

### SUMMARY OF THE INVENTION

The present invention provides CE1 family polypeptides having ferulic acid esterase and/or acetyl xylan esterase activity and polynucleotides encoding the polypeptides. The CE1 family polypeptides of the present invention release more monomeric arabinose and/or xylose when used in combination with polypeptides having arabinofuranosidase on di- and mono-substituted arabinose, polypeptides having xylanase activity, and polypeptides having beta-xylosidase activity. The addition of an alpha-xylosidase, for example a GH31 alpha-xylosidase, further increases the release of monomeric sugars. Surprisingly and unexpectedly, the compositions of the present invention significantly increase yields of monomeric arabinose and/or xylose without requiring alpha-glucuronidases, and the addition of alpha-xylosidases to the compositions further increases those yields.

SEQ ID NO: 1 is the nucleotide sequence encoding a wild-type Microsphaeropsis arundinis CE1 family polypeptide having ferulic acid esterase and/or acetyl xylan esterase activity of the invention.

SEQ ID NO: 2 is the full-length amino amino acid sequence of a wild-type Microsphaeropsis arundinis CE1 family polypeptide having ferulic acid esterase and/or acetyl xylan esterase activity of the invention.

SEQ ID NO: 3 is the mature polypeptide of a wild-type Microsphaeropsis arundinis CE1 family polypeptide having ferulic acid esterase and/or acetyl xylan esterase activity of the invention.

SEQ ID NO: 4 is the nucleotide sequence encoding a wild-type Microsphaeropsis arundinis CE1 family polypeptide having ferulic acid esterase and/or acetyl xylan esterase activity of the invention.

SEQ ID NO: 5 is the full-length amino amino acid sequence of a wild-type Microsphaeropsis arundinis CE1 family polypeptide having ferulic acid esterase and/or acetyl xylan esterase activity of the invention.

SEQ ID NO: 6 is the mature polypeptide of a wild-type Microsphaeropsis arundinis CE1 family polypeptide having ferulic acid esterase and/or acetyl xylan esterase activity of the invention.

SEQ ID NO: 7 is the nucleotide sequence encoding a wild-type Microsphaeropsis arundinis CE1 family polypeptide having ferulic acid esterase and/or acetyl xylan esterase activity of the invention.

SEQ ID NO: 8 is the full-length amino amino acid sequence of a wild-type Microsphaeropsis arundinis CE1 family polypeptide having ferulic acid esterase and/or acetyl xylan esterase activity of the invention.

SEQ ID NO: 9 is the mature polypeptide of a wild-type Microsphaeropsis arundinis CE1 family polypeptide having ferulic acid esterase and/or acetyl xylan esterase activity of the invention.

SEQ ID NO: 10 is the nucleotide sequence encoding a wild-type Microsphaeropsis arundinis CE1 family polypeptide having ferulic acid esterase and/or acetyl xylan esterase activity of the invention.

SEQ ID NO: 11 is the full-length amino amino acid sequence of a wild-type Microsphaeropsis arundinis CE1 family polypeptide having ferulic acid esterase and/or acetyl xylan esterase activity of the invention.

SEQ ID NO: 12 is the mature polypeptide of a wild-type Microsphaeropsis arundinis CE1 family polypeptide having ferulic acid esterase and/or acetyl xylan esterase activity of the invention.

SEQ ID NO: 13 is the nucleotide sequence encoding a wild-type Microsphaeropsis arundinis CE1 family polypeptide having ferulic acid esterase activity and/or acetyl xylan esterase of the invention.

SEQ ID NO: 14 is the full-length amino amino acid sequence of a wild-type Microsphaeropsis arundinis CE1 family polypeptide having ferulic acid esterase and/or acetyl xylan esterase activity of the invention.

SEQ ID NO: 15 is the mature polypeptide of a wild-type Microsphaeropsis arundinis CE1 family polypeptide having ferulic acid esterase and/or acetyl xylan esterase activity of the invention.

Accordingly, the present invention relates to polypeptides having ferulic acid esterase and/or acetyl xylan esterase activity selected from the group consisting of:
(i)
(a) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 2;
(b) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 3;
(c) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 2;
(d) a polypeptide encoded by a polynucleotide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 1;
(e) a polypeptide derived from SEQ ID NO: 2, a mature polypeptide of SEQ ID NO: 2, or SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity;
(ii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 5;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 6;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 5;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 4;
(e) a polypeptide derived from SEQ ID NO: 5, a mature polypeptide of SEQ ID NO: 5, or SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity;
(iii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 8;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 9;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 8;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 7;
(e) a polypeptide derived from SEQ ID NO: 8, a mature polypeptide of SEQ ID NO: 8, or SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity;
(iv)
(a) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 11;
(b) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 12;
(c) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 11;
(d) a polypeptide encoded by a polynucleotide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 10;
(e) a polypeptide derived from SEQ ID NO: 11, a mature polypeptide of SEQ ID NO: 11, or SEQ ID NO: 12 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity; and
(v)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 14;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 15;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 14;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 13;
(e) a polypeptide derived from SEQ ID NO: 14, a mature polypeptide of SEQ ID NO: 14, or SEQ ID NO: 15 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity.

The present invention also relates to polynucleotides encoding the polypeptides of the present invention; nucleic acid constructs; recombinant expression vectors; recombinant host cells comprising the polynucleotides; and methods of producing the polypeptides.

The present invention also relates to compositions comprising the CE1 family polypeptides and use of the compositions for solubilizing hemicellulosic fiber and increasing release of monomeric arabinose and/or xylose.

### BRIEF DESCRIPTION OF THE FIGURE

The FIGURE is an alignment of exemplary CE1 polypeptides of the present invention showing they share the conserved active site serine, histidine and aspartic acid residues that form the catalytic triad responsible for the canonical serine hydrolase mechanism and the conserved pentapeptide having the consensus sequence G-X-S-X-G.

### DEFINITIONS

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

Acetyl xylan esterase: The term “acetyl xylan esterase” means a polypeptide having acetyl xylan esterase activity (EC 3.1.1.72) that catalyzes the hydrolysis of acetyl groups from polymeric xylan, acetylated xylose, acetylated glucose, alpha-napthyl acetate, p-nitrophenyl acetate but not from triacylglycerol.

Acetyl xylan esterase activity: One Unit of acetyl xylan esterase activity is defined as the amount of enzyme required to release one μmole of p-nitrophenol per minute from 4-nitrophenyl acetate in 100 mM sodium citrate buffer, pH 5 at 40° C. 100 mM pNP-actate is dissolved in DMSO as substrates stock solution. The stock solution is diluted 50× in 100 mM sodium citrate to make 2 mM pNP-acetate substrate solution. 175 μl substrate solution and 25 ul diluted enzyme is mixed in 96-well plate and incubated at 37° C. The released ρ-nitrophenol is monitored at 410 nm by a spectrophotometer.

Alpha-L-arabinofuranosidase: “Alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinofuranosidase, alpha-arabinofuranosidase, alpha-L-arabinofuranosidase, alpha-arabinofuranosidase, polysaccharide alpha-L-arabinofuranosidase, alpha-L-arabinofuranoside hydrolase, L-arabinofuranosidase, or alpha-L-arabinanase.

Alpha-L-arabinofuranosidase Activity: For purposes of the present invention, alpha-L-arabinofuranosidase activity is determined using 5 mg of medium viscosity wheat arabinoxylan (Megazyme International Ireland, Ltd., Bray, Co. Wicklow, Ireland) per ml of 100 mM sodium acetate pH 5 in a total volume of 200 micro liters for 30 minutes at 40 degrees centigrade followed by arabinose analysis by AMINEX® HPX-87H column chromatography (Bio-Rad Laboratories, Inc., Hercules, CA, USA).

Alpha-xylosidase: “Alpha-xylosidase” means an alpha-D-xyloside xylohydrolase (EC 3.2.1.177) that catalyzes hydrolysis of a terminal, unsubstituted xyloside at the extreme reducing end of a xylogluco-oligosaccharide.

Alpha-xylosidase Activity: For purposes of the present invention, one unit of alpha-xylosidase is defined as 1.0 μmole of p-nitrophenolate anion produced per minute at 40 degrees centigrade, pH 5 from 1 mM p-nitrophenyl-alpha-D-xyloside as substrate in 100 mM sodium citrate containing 0.01 percent TWEEN® 20 in a total volume of 200 micro liters.

Beta-xylosidase: “Beta-xylosidase” means a beta-D-xyloside xylohydrolase (E.C. 3.2.1.37) that catalyzes the exo-hydrolysis of short beta (1-4)-xylooligosaccharides to remove successive D-xylose residues from non-reducing termini.

Beta-xylosidase Activity: For purposes of the present invention, one unit of beta-xylosidase is defined as 1.0 μmole of p-nitrophenolate anion produced per minute at 40 degrees centigrade, pH 5 from 1 mM p-nitrophenyl-beta-D-xyloside as substrate in 100 mM sodium citrate containing 0.01 percent TWEEN® 20 in a total volume of 200 micro liters.

Carbohydrate Esterease Family 1 (CE1): Carbohydrate Esterase Family 1 is abbreviated herein as “CE1” and encompasses acetyl xylan esterase (EC 3.1.1.72), cinnamoyl esterase (EC 3.1.1.-), feruloyl esterase (EC 3.1.1.73), S-formyl hydrolase (EC 3.1.2.12), diacylglycerol O-acyltransferase (EC 2.3.1.20); and trehalose 6-O-mycolytransferase (EC 2.3.1.122). The CE1 polypeptides of the present invention primarily have feruloyl esterase activity (EC 3.1.1.73) (referred to herein as “ferulic acid esterase activity”) and/or acetyl xylan esterase activity (EC 3.1.1.72).

Ferulic Acid Esterase: The term “ferulic acid esterase” means a 4-hydroxy-3-methoxycinnamoyl-sugar hydrolase (EC 3.1.1.73) that catalyzes the hydrolysis of the 4-hydroxy-3-methoxycinnamoyl (feruloyl) group from an esterified sugar, which is usually arabinose in ‘natural’ substrates.

Ferulic Acid Esterase Activity: One unit of ferulic acid esterase activity equals the amount of enzyme capable of releasing 1 micromole of p-nitrophenol per minute at pH 7, 37° C. 25 mM p-nitrophenyl ferulate is dissolved in DMSO, diluted by 100 mM PBS pH 7 containing 1% Tween-80 to 1 mM as substrate solution. 10 ul enzyme solution and 990 ul substrate solution is incubated at 37° C., 600 rpm on a thermomixer for 10 min. The released p-nitrophenol is monitored at 410 nm by a spectrophotometer.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification, and secretion.

Expression vector: An “expression vector” refers to a linear or circular DNA construct comprising a DNA sequence encoding a polypeptide, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a polypeptide, wherein the “extended” polypeptide has ferulic acid esterase and/or acetyl xylan esterase activity.

Fermentation product: “Fermentation product” means a product produced by a process including fermenting using a fermenting organism. Fermentation products include alcohols (e.g., ethanol, methanol, butanol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. In an embodiment the fermentation product is ethanol.

Fermenting organism: “Fermenting organism” refers to any organism, including bacterial and fungal organisms, especially yeast, suitable for use in a fermentation process and capable of producing the desired fermentation product.

Fragment: The term “fragment” means a polypeptide having one or more amino acids absent from the amino and/or carboxyl terminus of the mature polypeptide, wherein the fragment has ferulic acid esterase and/or acetyl xylan esterase activity.

Fusion polypeptide: The term “fusion polypeptide” is a polypeptide in which one polypeptide is fused at the N-terminus and/or the C-terminus of a polypeptide of the present invention. A fusion polypeptide is produced by fusing a polynucleotide encoding another polypeptide to a polynucleotide of the present invention, or by fusing two or more polynucleotides of the present invention together. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

GH3 beta-xylosidase: “GH3 beta-xylosidase” is an abbreviation for Glycoside Hydrolase Family 3 beta-xylosidases, which are xylan 1,4-beta-xylosidases (EC 3.2.1.37) that catalyze the hydrolysis (1→4)-β-D-xylans, to remove successive D-xylose residues from the non-reducing termini.

GH5 xylanase: “GH5 xylanase” is an abbreviation for Glycoside Hydrolase Family 5 xylanase, which consist primarily of endo-1,4-β-xylanases (EC 3.2.1.8) that catalyze the endohydrolysis of (1→4)-β-D-xylosidic linkages in xylans.

GH5_21 xylanase: “GH5_21 xylanase” is an abbreviation for Glycoside Hydrolase Family 5 subfamily 21 endo-beta-1,4-xylanases that possess a three-dimensional structure characterized by a (β/α) β barrel and use a glutamine residue as a catalytic nucleophile/base.

GH5_35 xylanase: “GH5_35 xylanase” is an abbreviation for Glycoside Hydrolase Family 5 subfamily 35 endo-beta-1,4-xylanases that possess a three-dimensional structure characterized by a (β/α) β barrel and use a glutamine residue as a catalytic nucleophile/base.

GH8 xylanase: “GH8 xylanase” is an abbreviation for Glycoside Hydrolase Family 8 xylanases, which consists of endo-1, 4-β-xylanases (EC 3.2.1.8) that catalyze the endohydrolysis of (1→4)-β-D-xylosidic linkages in xylans.

GH10 xylanase: “GH10 xylanase” is an abbreviation for Glycoside Hydrolase Family 10 xylanases, which consists of endo-1, 3-β-xylanases (EC 3.2.1.32) that catalyze the random endohydrolysis of (1→3)-β-D-glycosidic linkages in (1→3)-β-D-xylans, and endo-1, 4-β-xylanases (EC 3.2.1.8) that catalyze the endohydrolysis of (1→4)-β-D-xylosidic linkages in xylans.

GH11 xylanase: “GH11 xylanase” is an abbreviation for Glycoside Hydrolase Family 11 xylanase, which is an endo-β-1,4-xylanase (EC 3.2.1.8) that catalyzes the endohydrolysis of (1→4)-β-D-xylosidic linkages in xylans.

GH30_8 xylanase: “GH30_8 xylanase” is an abbreviation for Glycoside Hydrolase 30 subfamily 8 xylanases, which include endo-beta-1,4-xylanase (EC 3.2.1.8) that catalyze the endohydrolysis of (1→4)-β-D-xylosidic linkages in xylans and glucuronoarabinoxylan-specific endo-β-1,4-xylanases (EC 3.2.1.136) that catalyze the endohydrolysis of (1→4)-β-D-xylosyl links in some glucuronoarabinoxylans. endohydrolysis of (1→4)-β-D-xylosyl links in some glucuronoarabinoxylans.

GH31 alpha-xylosidase: “GH31 arabinofuranosidase” is an abbreviation for Glycoside Hydrolase Family 31 alpha-xylosidases, which is an alpha-D-xyloside xylohydrolase (EC 3.2.1.177) that catalyzes hydrolysis of a terminal, unsubstituted xyloside at the extreme reducing end of a xylogluco-oligosaccharide. Exemplary alpha-xylosidases from the GH31 family utilize a two-step, double-displacement mechanism employing a covalent glycosyl-enzyme intermediate, and produce a product with an anomeric configuration.

GH43 arabinofuranosidase: “GH43 arabinofuranosidase” is an abbreviation for Glycoside Hydrolase Family 43 arabinofuranosidase, which is an alpha-L-arabinofuranosidase (EC 3.2.1.55) that catalyzes the hydrolysis of terminal non-reducing alpha-L-arabinofuranoside residues in alpha-L-arabinosides.

GH51 arabinofuranosidase: “GH51 arabinofuranosidase” is an abbreviation for Glycoside Hydrolase Family 51 arabinofuranosidase, which is an alpha-L-arabinofuranosidase (EC 3.2.1.55) that catalyzes the hydrolysis of terminal non-reducing alpha-L-arabinofuranoside residues in alpha-L-arabinosides.

Initial gelatinization temperature: “Initial gelatinization temperature” means the lowest temperature at which gelatinization of the starch commences. Starch heated in water begins to gelatinize between 50 degrees centigrade and 75 degrees C.; the exact temperature of gelatinization depends on the specific starch, and can readily be determined by the skilled artisan. Thus, the initial gelatinization temperature may vary according to the plant species, to the particular variety of the plant species as well as with the growth conditions. In the context of this disclosure the initial gelatinization temperature of a given starch-containing grain is the temperature at which birefringence is lost in 5 percent of the starch granules using the method described by Gorinstein. S. and Lii. C, Starch/Starke, Vol. 44 (12) pp. 461-466 (1992).

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Introduced: The term “introduced” in the context of inserting a nucleic acid sequence into a cell, means “transfection”, “transformation” or “transduction,” as known in the art.

Isolated: The term “isolated” means a polypeptide, nucleic acid, cell, or other specified material or component that has been separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted polypeptide expressed in a host cell.

Mature polypeptide: The term “mature polypeptide” means a polypeptide in its mature form following N-terminal and/or C-terminal processing (e.g., removal of signal peptide). In one aspect, the mature polypeptide is amino acids 20 to 295 of SEQ ID NO: 2. In one aspect, the mature polypeptide is SEQ ID NO: 3. In one aspect, the mature polypeptide is amino acids 25 to 282 of SEQ ID NO: 5. In one aspect, the mature polypeptide is SEQ ID NO: 6. In one aspect, the mature polypeptide is amino acids 21 to 310 of SEQ ID NO: 8. In one aspect, the mature polypeptide is SEQ ID NO: 9. In one aspect, the mature polypeptide is amino acids 19 to 302 of SEQ ID NO: 11. In one aspect, the mature polypeptide is SEQ ID NO: 12. In one aspect, the mature polypeptide is amino acids 20 to 286 of SEQ ID NO: 14. In one aspect, the mature polypeptide is SEQ ID NO: 15.

Mature polypeptide coding sequence: The term “mature polypeptide coding sequence” means a polynucleotide that encodes a mature polypeptide having ferulic acid esterase and/or acetyl xylan esterase activity. In one aspect, the mature polypeptide coding sequence is nucleotides 60 to 885 of SEQ ID NO: 1. In one aspect, the mature polypeptide coding sequence is nucleotides 75 to 846 of SEQ ID NO: 4. In one aspect, the mature polypeptide coding sequence is nucleotides 63 to 930 of SEQ ID NO: 7. In one aspect, the mature polypeptide coding sequence is nucleotides 57 to 906 of SEQ ID NO: 10. In one aspect, the mature polypeptide coding sequence is nucleotides 60 to 858 of SEQ ID NO: 13.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Purified: The term “purified” means a nucleic acid, polypeptide or cell that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

In one aspect, the term “purified” as used herein refers to the polypeptide or cell being essentially free from components (especially insoluble components) from the production organism. In other aspects. the term “purified” refers to the polypeptide being essentially free of insoluble components (especially insoluble components) from the native organism from which it is obtained. In one aspect, the polypeptide is separated from some of the soluble components of the organism and culture medium from which it is recovered. The polypeptide may be purified (i.e., separated) by one or more of the unit operations filtration, precipitation, or chromatography.

Accordingly, the polypeptide may be purified such that only minor amounts of other proteins, in particular, other polypeptides, are present. The term “purified” as used herein may refer to removal of other components, particularly other proteins and most particularly other enzymes present in the cell of origin of the polypeptide. The polypeptide may be “substantially pure”, i.e., free from other components from the organism in which it is produced, e.g., a host organism for recombinantly produced polypeptide. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein. a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The polypeptide of the present invention is preferably in a substantially pure form (i.e., the preparation is essentially free of other polypeptide material with which it is natively or recombinantly associated). This can be accomplished, for example by preparing the polypeptide by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, polypeptide or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g. depth filtration (by use of filter aids or packed filter medias, cloth filtration in chamber filters, rotary-drum filtration, drum filtration, rotary vacuum-drum filters, candle filters, horizontal leaf filters or similar, using sheet or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hydro cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”. The sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), version 6.6.0. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

$$( Identical Residues \times 100 ) / ( Length of Alignment - Total Number of Gaps in Alignment )$$

The sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), version 6.6.0. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

$$( Identical Deoxyribonucleotides \times 100 ) / ( Length of Alignment - Total Number of Gaps in Alignment )$$

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Subsequence: The term “subsequence” means a polynucleotide having one or more nucleotides absent from the 5′ and/or 3′ end of a mature polypeptide coding sequence; wherein the subsequence encodes a fragment having ferulic acid esterase and/or acetyl xylan esterase activity.

Thermostable: “Thermostable” means the enzyme is not denatured or deactivated when it is used in a liquefaction step of a process of the invention. In other words, a thermostable enzyme is suitable for liquefaction if it has a denaturation temperature (Td) that is compatible with the liquefaction temperature and retains its activity at that temperature.

Thin Stillage: “Thin Stillage” refers to centrate separated from whole stillage that is pumped toward the evaporators to be concentrated into syrup.

Variant: The term “variant” means a polypeptide having ferulic acid esterase and/or acetyl xylan esterase activity comprising a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Whole Stillage: “Whole stillage” includes the material that remains at the end of the distillation process after recovery of the fermentation product, e.g., ethanol.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

Xylanase: “Xylanase” encompasses endo-1,4-β-xylanases (EC 3.2.1.8) that catalyze the endohydrolysis of (1→4)-β-D-xylosidic linkages in xylans and glucuronoarabinoxylan endo-1,4-beta-xylanases (E.C. 3.2.1.136) that catalyze the endohydrolysis of 1,4-beta-D-xylosyl links in some glucuronoarabinoxylans.

Xylanase Activity: Activity of EC 3.2.1.8 xylanases can be determined using birchwood xylan as substrate. One unit of xylanase is defined as 1.0 μmole of reducing sugar (measured in glucose equivalents as described by Lever, 1972, A new reaction for colorimetric determination of carbohydrates, Anal. Biochem 47: 273-279) produced per minute during the initial period of hydrolysis at 50° C., pH 5 from 2 g of birchwood xylan per liter as substrate in 50 mM sodium acetate containing 0.01% TWEEN® 2. Activity of EC 3.2.1.136 xylanases can be determined with 0.2% AZCL-glucuronoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-glucuronoxylan as substrate in 200 mM sodium phosphate pH 6.

### DETAILED DESCRIPTION OF THE INVENTION

Carbohydrate Esterase Family 1 (CE1) Polypeptides Having Ferulic Acid Esterase and/or Acetyl Xylan Esterase Activity

The present invention relates to carbohydrate esterase family 1 (CE1) polypeptides having ferulic acid esterase and/or acetyl xylan esterase activity. In an aspect, the invention relates to polypeptides having ferulic acid esterase and/or acetyl xylan esterase activity, selected from the group consisting of:
(a) a polypeptide having at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 2;
(b) a polypeptide having at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3;
(c) a polypeptide having at least 83%, at least 84%, at least 85%, at least 86%, at least 87%, at least 88%, at least 89%, at least 90%, at least 91%, at least 92%, at least 93% identity, at least 94%, at least 95%, at least 96%, at least 97%, at least 98%, or at least 99% sequence identity to a mature polypeptide of SEQ ID NO: 2;
(d) a polypeptide encoded by a polynucleotide having at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 1;
(e) a polypeptide derived from SEQ ID NO: 2, a mature polypeptide of SEQ ID NO: 2, or SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity.

In an aspect, the polypeptide has a sequence identity of at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 2 or a mature polypeptide of SEQ ID NO: 2.

In another aspect, the polypeptide has a sequence identity of at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 3.

The polypeptide preferably comprises, consists essentially of, or consists of the amino acid sequence of SEQ ID NO: 2 or a mature polypeptide thereof.

The polypeptide preferably comprises, consists essentially of, or consists of amino acids 20 to 295 of SEQ ID NO: 2.

The polypeptide preferably comprises, consists essentially of, or consists of the amino acid sequence of SEQ ID NO: 3.

The polypeptide may have an N-terminal and/or C-terminal extension of one or more amino acids, e.g., 1-5 amino acids.

In another aspect, the polypeptide is a fragment containing at least 235 amino acid residues (e.g., amino acids 1 to 235 of SEQ ID NO: 3), at least 248 amino acid residues (e.g., amino acids 1 to 248 of SEQ ID NO: 3), or at least 262 amino acid residues (e.g., amino acids 1 to 262 of SEQ ID NO: 3).

In some embodiments, the polypeptide is encoded by a polynucleotide having a sequence identity of at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 1.

The polynucleotide encoding the polypeptide preferably comprises, consists essentially of, or consists of nucleotides 60 to 885 of SEQ ID NO: 1.

In another aspect, the polypeptide is derived from SEQ ID NO: 2 by substitution, deletion or addition of one or several amino acids. In another aspect, the polypeptide is derived from a mature polypeptide of SEQ ID NO: 2 by substitution, deletion or addition of one or several amino acids. In another aspect, the polypeptide is derived from SEQ ID NO: 3 by substitution, deletion or addition of one or more amino acids. In some embodiments, the polypeptide is a variant of SEQ ID NO: 3 comprising a substitution, deletion, and/or insertion at one or more positions. In one aspect, the number of amino acid substitutions, deletions and/or insertions introduced into the polypeptide of SEQ ID NO: 3 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In an aspect, the invention relates to polypeptides having ferulic acid esterase and/or acetyl xylan esterase activity, selected from the group consisting of:
(a) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 5;
(b) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 6;
(c) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a mature polypeptide of SEQ ID NO: 5;
(d) a polypeptide encoded by a polynucleotide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 4;
(e) a polypeptide derived from SEQ ID NO: 5, a mature polypeptide of SEQ ID NO: 5, or SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity.

In an aspect, the polypeptide has a sequence identity of at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 5 or a mature polypeptide of SEQ ID NO: 5.

In another aspect, the polypeptide has a sequence identity of at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 6.

The polypeptide preferably comprises, consists essentially of, or consists of the amino acid sequence of SEQ ID NO: 5 or a mature polypeptide thereof.

The polypeptide preferably comprises, consists essentially of, or consists of amino acids 25 to 282 of SEQ ID NO: 5.

The polypeptide preferably comprises, consists essentially of, or consists of the amino acid sequence of SEQ ID NO: 6.

The polypeptide may have an N-terminal and/or C-terminal extension of one or more amino acids, e.g., 1-5 amino acids.

In another aspect, the polypeptide is a fragment containing at least 219 amino acid residues (e.g., amino acids 1 to 219 of SEQ ID NO: 6), at least 232 amino acid residues (e.g., amino acids 1 to 232 of SEQ ID NO: 6), or at least 245 amino acid residues (e.g., amino acids 1 to 245 of SEQ ID NO: 6).

In some embodiments, the polypeptide is encoded by a polynucleotide having a sequence identity of at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 4.

The polynucleotide encoding the polypeptide preferably comprises, consists essentially of, or consists of nucleotides 75 to 846 of SEQ ID NO: 4.

In another aspect, the polypeptide is derived from SEQ ID NO: 5 by substitution, deletion or addition of one or several amino acids. In another aspect, the polypeptide is derived from a mature polypeptide of SEQ ID NO: 5 by substitution, deletion or addition of one or several amino acids. In another aspect, the polypeptide is derived from SEQ ID NO: 6 by substitution, deletion or addition of one or more amino acids. In some embodiments, the polypeptide is a variant of SEQ ID NO: 6 comprising a substitution, deletion, and/or insertion at one or more positions. In one aspect, the number of amino acid substitutions, deletions and/or insertions introduced into the polypeptide of SEQ ID NO: 6 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In an aspect, the invention relates to polypeptides having ferulic acid esterase and/or acetyl xylan esterase activity, selected from the group consisting of:
(a) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 8;
(b) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 9;
(c) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a mature polypeptide of SEQ ID NO: 8;
(d) a polypeptide encoded by a polynucleotide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7;
(e) a polypeptide derived from SEQ ID NO: 8, a mature polypeptide of SEQ ID NO: 8, or SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity.

In an aspect, the polypeptide has a sequence identity of at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 8 or a mature polypeptide of SEQ ID NO: 8.

In another aspect, the polypeptide has a sequence identity of at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 9.

The polypeptide preferably comprises, consists essentially of, or consists of the amino acid sequence of SEQ ID NO: 8 or a mature polypeptide thereof.

The polypeptide preferably comprises, consists essentially of, or consists of amino acids 21 to 310 of SEQ ID NO: 8.

The polypeptide preferably comprises, consists essentially of, or consists of the amino acid sequence of SEQ ID NO: 9.

The polypeptide may have an N-terminal and/or C-terminal extension of one or more amino acids, e.g., 1-5 amino acids.

In another aspect, the polypeptide is a fragment containing at least 247 amino acid residues (e.g., amino acids 1 to 247 of SEQ ID NO: 9), at least 261 amino acid residues (e.g., amino acids 1 to 261 of SEQ ID NO: 9), or at least 276 amino acid residues (e.g., amino acids 1 to 276 of SEQ ID NO: 9).

In some embodiments, the polypeptide is encoded by a polynucleotide having a sequence identity of at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 7.

The polynucleotide encoding the polypeptide preferably comprises, consists essentially of, or consists of nucleotides 63 to 930 of SEQ ID NO: 7.

In another aspect, the polypeptide is derived from SEQ ID NO: 8 by substitution, deletion or addition of one or several amino acids. In another aspect, the polypeptide is derived from a mature polypeptide of SEQ ID NO: 8 by substitution, deletion or addition of one or several amino acids. In another aspect, the polypeptide is derived from SEQ ID NO: 9 by substitution, deletion or addition of one or more amino acids. In some embodiments, the polypeptide is a variant of SEQ ID NO: 9 comprising a substitution, deletion, and/or insertion at one or more positions. In one aspect, the number of amino acid substitutions, deletions and/or insertions introduced into the polypeptide of SEQ ID NO: 9 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In an aspect, the invention relates to polypeptides having ferulic acid esterase activity and/or acetyl xylan esterase activity, selected from the group consisting of:
(a) a polypeptide having at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 11;
(b) a polypeptide having at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 12;
(c) a polypeptide having at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a mature polypeptide of SEQ ID NO: 11;
(d) a polypeptide encoded by a polynucleotide having at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 10;
(e) a polypeptide derived from SEQ ID NO: 11, a mature polypeptide of SEQ ID NO: 11, or SEQ ID NO: 12 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity.

In an aspect, the polypeptide has a sequence identity of at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 11 or a mature polypeptide of SEQ ID NO: 11.

In another aspect, the polypeptide has a sequence identity of at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 12.

The polypeptide preferably comprises, consists essentially of, or consists of the amino acid sequence of SEQ ID NO: 11 or a mature polypeptide thereof.

The polypeptide preferably comprises, consists essentially of, or consists of amino acids 19 to 302 of SEQ ID NO: 11 or a mature polypeptide thereof.

The polypeptide preferably comprises, consists essentially of, or consists of the amino acid sequence of SEQ ID NO: 12.

The polypeptide may have an N-terminal and/or C-terminal extension of one or more amino acids, e.g., 1-5 amino acids.

In another aspect, the polypeptide is a fragment containing at least 234 amino acid residues (e.g., amino acids 1 to 234 of SEQ ID NO: 12), at least 255 amino acid residues (e.g., amino acids 1 to 255 of SEQ ID NO: 12), or at least 270 amino acid residues (e.g., amino acids 1 to 270 of SEQ ID NO: 12).

In some embodiments, the polypeptide is encoded by a polynucleotide having a sequence identity of at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 10.

The polynucleotide encoding the polypeptide preferably comprises, consists essentially of, or consists of nucleotides 57 to 906 of SEQ ID NO: 10.

In another aspect, the polypeptide is derived from SEQ ID NO: 11 by substitution, deletion or addition of one or several amino acids. In another aspect, the polypeptide is derived from a mature polypeptide of SEQ ID NO: 11 by substitution, deletion or addition of one or several amino acids. In another aspect, the polypeptide is derived from SEQ ID NO: 12 by substitution, deletion or addition of one or more amino acids. In some embodiments, the polypeptide is a variant of SEQ ID NO: 12 comprising a substitution, deletion, and/or insertion at one or more positions. In one aspect, the number of amino acid substitutions, deletions and/or insertions introduced into the polypeptide of SEQ ID NO: 12 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In an aspect, the invention relates to polypeptides having ferulic acid esterase and/or acetyl xylan esterase activity, selected from the group consisting of:
(a) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 14;
(b) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 15;
(c) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a mature polypeptide of SEQ ID NO: 14;
(d) a polypeptide encoded by a polynucleotide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 13;
(e) a polypeptide derived from SEQ ID NO: 14, a mature polypeptide of SEQ ID NO: 14, or SEQ ID NO: 15 by substitution, deletion or addition of one or several amino acids;
(f) a polypeptide derived from the polypeptide of (a), (b), (c), (d) or (e) wherein the N- and/or C-terminal end has been extended by the addition of one or more amino acids; and
(g) a fragment of the polypeptide of (a), (b), (c), (d), or (e);
wherein the polypeptide has ferulic acid esterase and/or acetyl xylan esterase activity.

In an aspect, the polypeptide has a sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% to SEQ ID NO: 14 or a mature polypeptide of SEQ ID NO: 14.

In another aspect, the polypeptide has a sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% to SEQ ID NO: 15.

The polypeptide preferably comprises, consists essentially of, or consists of the amino acid sequence of SEQ ID NO: 14 or a mature polypeptide thereof.

The polypeptide preferably comprises, consists essentially of, or consists of amino acids 20 to 286 of SEQ ID NO: 14.

The polypeptide preferably comprises, consists essentially of, or consists of the amino acid sequence of SEQ ID NO: 15.

The polypeptide may have an N-terminal and/or C-terminal extension of one or more amino acids, e.g., 1-5 amino acids.

In another aspect, the polypeptide is a fragment containing at least 227 amino acid residues (e.g., amino acids 1 to 227 of SEQ ID NO: 15), at least 240 amino acid residues (e.g., amino acids 1 to 240 of SEQ ID NO: 15), or at least 254 amino acid residues (e.g., amino acids 1 to 254 of SEQ ID NO: 15).

In some embodiments, the polypeptide is encoded by a polynucleotide having a sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% to the mature polypeptide coding sequence of SEQ ID NO: 13.

The polynucleotide encoding the polypeptide preferably comprises, consists essentially of, or consists of nucleotides 60 to 858 of SEQ ID NO: 13.

In another aspect, the polypeptide is derived from SEQ ID NO: 14 by substitution, deletion or addition of one or several amino acids. In another aspect, the polypeptide is derived from a mature polypeptide of SEQ ID NO: 14 by substitution, deletion or addition of one or several amino acids. In another aspect, the polypeptide is derived from SEQ ID NO: 15 by substitution, deletion or addition of one or more amino acids. In some embodiments, the polypeptide is a variant of SEQ ID NO: 15 comprising a substitution, deletion, and/or insertion at one or more positions. In one aspect, the number of amino acid substitutions, deletions and/or insertions introduced into the polypeptide of SEQ ID NO: 15 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for ferulic acid esterase and/or acetyl xylan esterase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

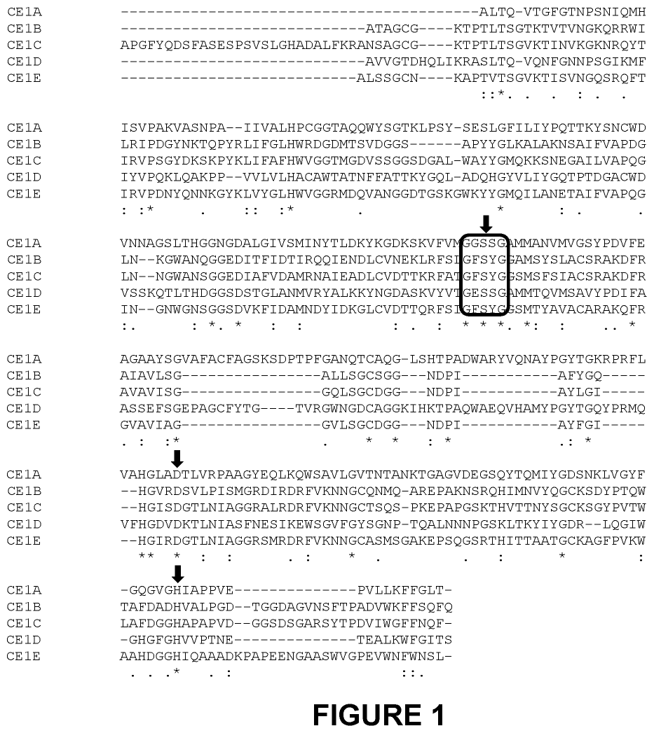

Carbohydrate esterase family 1 (CE1) polypeptides act on various substrates using the canonical serine hydrolase mechanism that involves a catalytic triad comprising a nucleophilic serine, a histidine, and an acidic amino acid, such as aspartic acid and glutamic acid. The catalytic serine is situated at the center of a conserved pentapeptide having a consensus sequence G-X-S-X-G. The pentapeptide segment establishes a “nucleophilic elbow”, which is a fingerprint used to identify polypeptides of the CE1 family based on their primary structure. The histidine is conserved, whereas the acid residue of the catalytic triad may be an aspartic acid or glutamic acid. The location of these features is shown in Table 1 below and an alignment showing conservation of these features in the mature sequences of the exemplary CE1 polypeptides of the present invention is shown in FIG. 1.

TABLE 1

| | Active | Active | Active | Site of G-X- |
|---|---|---|---|---|
| | site_S | site_D | site_H | S-X-G |
| CE1A | 117 | 201 | 260 | 115 . . . 119 |
| CE1B | 122 | 170 | 229 | 120 . . . 124 |
| CE1C | 155 | 203 | 262 | 153 . . . 157 |
| CE1D | 130 | 211 | 267 | 128 . . . 132 |
| CE1E | 129 | 177 | 237 | 127 . . . 131 |

The polypeptide may be a fusion polypeptide.

In an aspect, the polypeptide is isolated.

In another aspect, the polypeptide is purified.

Sources of Polypeptides Having Ferulic Acid Esterase and/or Acetyl Xylan Esterase Activity

A polypeptide having ferulic acid esterase and/or acetyl xylan esterase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide of the invention has been inserted. In one aspect, the polypeptide obtained from a given source is secreted extracellularly.

In an aspect, the polypeptide is obtained from a Microsphaeropsis. In another aspect, the polypeptide is a polypeptide obtained from a Microsphaeropsis amaranthi. In another aspect, the polypeptide is a polypeptide obtained from a Microsphaeropsis arundinis. In another aspect, the polypeptide is a polypeptide obtained from a Microsphaeropsis fusca. In another aspect, the polypeptide is a polypeptide obtained from a Microsphaeropsis hellebori. In another aspect, the polypeptide is a polypeptide obtained from a Microsphaeropsis olivacea. In another aspect, the polypeptide is a polypeptide obtained from a Microsphaeropsis ononidicola. In another aspect, the polypeptide is a polypeptide obtained from a Microsphaeropsis proteae. In another aspect, the polypeptide is a polypeptide obtained from a Microsphaeropsis spartiij-uncei.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

The polypeptides may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Davis et al., 2012, Basic Methods in Molecular Biology, Elsevier).

### Polynucleotides

The present invention also relates to polynucleotides encoding a polypeptide of the present invention, as described herein.

The polynucleotide may be a genomic DNA, a cDNA, a synthetic DNA, a synthetic RNA, a mRNA, or a combination thereof. The polynucleotide may be cloned from a strain of Microsphaeropsis, or a related organism and thus, for example, may be a polynucleotide sequence encoding a variant of the polypeptide of the invention.

In an embodiment, the polynucleotide is a subsequence encoding a fragment having ferulic acid esterase and/or acetyl xylan esterase activity of the present invention. In an aspect, the subsequence contains at least 705 nucleotides (e.g., nucleotides 60 to 765 of SEQ ID NO: 1), at least 744 nucleotides (e.g., nucleotides 60 to 804 of SEQ ID NO: 1), or at least 786 nucleotides (e.g., nucleotides 60 to 846 of SEQ ID NO: 1).

In an embodiment, the polynucleotide is a subsequence encoding a fragment having ferulic acid esterase and/or acetyl xylan esterase activity of the present invention. In an aspect, the subsequence contains at least 657 nucleotides (e.g., nucleotides 75 to 732 of SEQ ID NO: 4), at least 696 nucleotides (e.g., nucleotides 75 to 771 of SEQ ID NO: 4), or at least 735 nucleotides (e.g., nucleotides 75 to 810 of SEQ ID NO: 4).

In an embodiment, the polynucleotide is a subsequence encoding a fragment having ferulic acid esterase and/or acetyl xylan esterase activity of the present invention. In an aspect, the subsequence contains at least 741 nucleotides (e.g., nucleotides 63 to 804 of SEQ ID NO: 7), at least 783 nucleotides (e.g., nucleotides 63 to 846 of SEQ ID NO: 7), or at least 828 nucleotides (e.g., nucleotides 63 to 891 of SEQ ID NO: 7).

In an embodiment, the polynucleotide is a subsequence encoding a fragment having ferulic acid esterase and/or acetyl xylan esterase activity of the present invention. In an aspect, the subsequence contains at least 702 nucleotides (e.g., nucleotides 57 to 759 of SEQ ID NO: 10), at least 765 nucleotides (e.g., nucleotides 57 to 822 of SEQ ID NO: 10), or at least 810 nucleotides (e.g., nucleotides 57 to 867 of SEQ ID NO: 10).

In an embodiment, the polynucleotide is a subsequence encoding a fragment having ferulic acid esterase and/or acetyl xylan esterase activity of the present invention. In an aspect, the subsequence contains at least 681 nucleotides (e.g., nucleotides 60 to 741 of SEQ ID NO: 13), at least 720 nucleotides (e.g., nucleotides 60 to 780 of SEQ ID NO: 13), or at least 762 nucleotides (e.g., nucleotides 60 to 822 of SEQ ID NO: 13).

In one embodiment the polynucleotide encoding the polypeptide of the present invention is isolated from a Microsphaeropsis cell.

The polynucleotide may also be mutated by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

In an aspect, the polynucleotide is isolated.

In another aspect, the polynucleotide is purified.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. Techniques for modifying polynucleotides utilizing recombinant DNA methods are well known in the art.

### Promoters

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, supra, and Song et al., 2016, PLOS One 11(7): e0158447.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenböck, 2016, “Gene Expression Systems in Fungi: Advancements and Applications”, Fungal Biology.

For expression in a yeast host, examples of useful promoters are described by Smolke et al., 2018, “Synthetic Biology: Parts, Devices and Applications” (Chapter 6: Constitutive and Regulated Promoters in Yeast: How to Design and Make Use of Promoters in S. cerevisiae), and by Schmoll and Dattenböck, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

Preferred terminators for yeast host cells may be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8: 423-488.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

### Leader Sequences

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Suitable leaders for bacterial host cells are described by Hambraeus et al., 2000, Microbiology 146(12): 3051-3059, and by Kaberdin and Bläsi, 2006, FEMS Microbiol. Rev. 30(6): 967-979.

Preferred leaders for filamentous fungal host cells may be obtained from the genes for Aspergillus oryzae TAKA amylase and Aspergillus nidulans triose phosphate isomerase.

Suitable leaders for yeast host cells may be obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

### Polyadenylation Sequences

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

### Signal Peptides

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. Any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Freudl, 2018, Microbial Cell Factories 17: 52.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40: 949-955

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

### Propeptides

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence. Additionally or alternatively, when both signal peptide and propeptide sequences are present, the polypeptide may comprise only a part of the signal peptide sequence and/or only a part of the propeptide sequence. Alternatively, the final or isolated polypeptide may comprise a mixture of mature polypeptides and polypeptides which comprise, either partly or in full length, a propeptide sequence and/or a signal peptide sequence.

### Regulatory Sequences

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In fungal systems, these regulatory sequences include the dihydrofolate reductase gene that is amplified in the presence of methotrexate, and the metallothionein genes that are amplified with heavy metals.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52: 7-23, as well in Balleza et al., 2009, FEMS Microbiol. Rev. 33(1): 133-151.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells, comprising a polynucleotide of the present invention operably linked to one or more control sequences that direct the production of a polypeptide of the present invention.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source. The polypeptide can be native or heterologous to the recombinant host cell. Also, at least one of the one or more control sequences can be heterologous to the polynucleotide encoding the polypeptide. The recombinant host cell may comprise a single copy, or at least two copies, e.g., three, four, five, or more copies of the polynucleotide of the present invention.

The host cell may be any microbial cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis and Bacillus subtilis cell.

For purposes of this invention, Bacillus classes/genera/species shall be defined as described in Patel and Gupta, 2020, Int. J. Syst. Evol. Microbiol. 70: 406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98: 6289-6294, Choi et al., 2006, J. Microbiol. Methods 64: 391-397, and Donald et al., 2013, J. Bacteriol. 195(11): 2612-2620.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16: 168 and procedures described in EP 238023, Yelton et al., 1984, Proc. Natl. Acad. Sci. USA 81: 1470-1474, Christensen et al., 1988, Bio/Technology 6: 1419-1422, and Lubertozzi and Keasling, 2009, Biotechn. Advances 27: 53-75. However, any method known in the art for introducing DNA into a fungal host cell can be used, and the DNA can be introduced as linearized or as circular polynucleotide.

The fungal host cell may be a yeast cell. “Yeast” as used herein includes ascosporogenous yeast (Endomycetales), basidiosporogenous yeast, and yeast belonging to the Fungi Imperfecti (Blastomycetes). For purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. In a preferred embodiment, the yeast host cell is a Pichia or Komagataella cell, e.g., a Pichia pastoris cell (Komagataella phaffii).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In an aspect, the host cell is isolated.

In another aspect, the host cell is purified.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Microsphaeropsis cell. In another aspect, the cell is a Microsphaeropsis amaranthi cell. In another aspect, the cell is a Microsphaeropsis arundinis cell. In another aspect, the cell is Microsphaeropsis arundinis. In another aspect, the cell is a Microsphaeropsis fusca cell. In another aspect, the cell is a Microsphaeropsis hellebore cell. In another aspect, the cell is a Microsphaeropsis olivacea cell. In another aspect, the cell is a Microsphaeropsis ononidicola cell. In another aspect, the cell is a Microsphaeropsis proteae cell. In another aspect, th the cell is a Microsphaeropsis spartii-juncei cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cell is cultivated in a nutrient medium suitable for production of the polypeptide using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptide, including, but not limited to, the use of specific antibodies, formation of an enzyme product, disappearance of an enzyme substrate, or an assay determining the relative or specific activity of the polypeptide.

The polypeptide may be recovered from the medium using methods known in the art, including, but not limited to, collection, centrifugation, filtration, extraction, spray-drying, evaporation, or precipitation. In one aspect, a whole fermentation broth comprising the polypeptide is recovered. In another aspect, a cell-free fermentation broth comprising the polypeptide is recovered.

The polypeptide may be purified by a variety of procedures known in the art to obtain substantially pure polypeptides and/or polypeptide fragments (see, e.g., Wingfield, 2015, Current Protocols in Protein Science; 80(1): 6.1.1-6.1.35; Labrou, 2014, Protein Downstream Processing, 1129: 3-10).

In an alternative aspect, the polypeptide is not recovered.

### Granules

The present invention also relates to enzyme granules/particles comprising a polypeptide of the invention. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm. The core diameter, measured as equivalent spherical diameter, can be determined using laser diffraction, such as using a Malvern Mastersizer and/or the method described under ISO13320 (2020).

In an embodiment, the core comprises a CE1 family polypeptide having ferulic acid esterase and/or acetyl xylan esterase activity of the present invention.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the polypeptide absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit has few or no uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄-7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Vol. 1; 1980; Elsevier. Preparation methods include known feed and granule formulation technologies, e.g.,
(a) Spray dried products, wherein a liquid polypeptide-containing solution is atomized in a spray drying tower to form small droplets which during their way down the drying tower dry to form a polypeptide-containing particulate material. Very small particles can be produced this way (Michael S. Showell (editor); Powdered detergents; Surfactant Science Series; 1998; Vol. 71; pages 140-142; Marcel Dekker).
(b) Layered products, wherein the polypeptide is coated as a layer around a pre-formed inert core particle, wherein a polypeptide-containing solution is atomized, typically in a fluid bed apparatus wherein the pre-formed core particles are fluidized, and the polypeptide-containing solution adheres to the core particles and dries up to leave a layer of dry polypeptide on the surface of the core particle. Particles of a desired size can be obtained this way if a useful core particle of the desired size can be found. This type of product is described in, e.g., WO 97/23606.
(c) Absorbed core particles, wherein rather than coating the polypeptide as a layer around the core, the polypeptide is absorbed onto and/or into the surface of the core. Such a process is described in WO 97/39116.
(d) Extrusion or pelletized products, wherein a polypeptide-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the polypeptide paste, which is harmful to the polypeptide (Michael S. Showell (editor); Powdered detergents; Surfactant Science Series; 1998; Vol. 71; pages 140-142; Marcel Dekker).
(e) Prilled products, wherein a polypeptide-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; Vol. 71; pages 140-142; Marcel Dekker). The product obtained is one wherein the polypeptide is uniformly distributed throughout an inert material instead of being concentrated on its surface. U.S. Pat. Nos. 4,016,040 and 4,713,245 describe this technique.
(f) Mixer granulation products, wherein a polypeptide-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the polypeptide. Such a process is described in U.S. Pat. No. 4,106,991, EP 170360, EP 304332, EP 304331, WO 90/09440 and WO 90/09428. In a particular aspect of this process, various high-shear mixers can be used as granulators. Granulates consisting of polypeptide, fillers and binders etc. are mixed with cellulose fibers to reinforce the particles to produce a so-called T-granulate. Reinforced particles, are more robust, and release less enzymatic dust.
(g) Size reduction, wherein the cores are produced by milling or crushing of larger particles, pellets, tablets, briquettes etc. containing the polypeptide. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.
(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.
(i) The cores may be subjected to drying, such as in a fluid bed drier. Other known methods for drying granules in the feed or enzyme industry can be used by the skilled person. The drying preferably takes place at a product temperature of from 25 to 90° C. For some polypeptides, it is important the cores comprising the polypeptide contain a low amount of water before coating with the salt. If water sensitive polypeptides are coated with a salt before excessive water is removed, the excessive water will be trapped within the core and may affect the activity of the polypeptide negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further comprise one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of polypeptides by the use of co-granulates is disclosed in WO 2013/188331.

The present invention also relates to protected polypeptides prepared according to the method disclosed in EP 238216.

### Liquid Formulations

The present invention also relates to liquid compositions comprising a polypeptide of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to, water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the liquid formulation comprises 20-80% w/w of polyol. In one embodiment, the liquid formulation comprises 0.001-2% w/w preservative.

In another embodiment, the invention relates to liquid formulations comprising:
(A) 0.001-25% w/w of a polypeptide having ferulic acid esterase and/or acetyl xylan esterase activity of the present invention;
(B) 20-80% w/w of polyol;
(C) optionally 0.001-2% w/w preservative; and
(D) water.

In another embodiment, the invention relates to liquid formulations comprising:
(A) 0.001-25% w/w of a polypeptide having ferulic acid esterase and/or acetyl xylan esterase activity of the present invention;
(B) 0.001-2% w/w preservative;
(C) optionally 20-80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20-80% polyol (i.e., total amount of polyol), e.g., 25-75% polyol, 30-70% polyol, 35-65% polyol, or 40-60% polyol. In one embodiment, the liquid formulation comprises 20-80% polyol, e.g., 25-75% polyol, 30-70% polyol, 35-65% polyol, or 40-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20-80% polyol (i.e., total amount of polyol), e.g., 25-75% polyol, 30-70% polyol, 35-65% polyol, or 40-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02-1.5% w/w preservative, e.g., 0.05-1% w/w preservative or 0.1-0.5% w/w preservative. In one embodiment, the liquid formulation comprises 0.001-2% w/w preservative (i.e., total amount of preservative), e.g., 0.02-1.5% w/w preservative, 0.05-1% w/w preservative, or 0.1-0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Compositions

The present invention relates to compositions comprising a carbohydrate esterase family 1 (CE1) polypeptide having ferulic acid esterase and/or acetyl xylan esterase activity, a polypeptide having arabinofuranosidase activity on disubstituted arabinose, a polypeptide having arabinofuranosidase activity on monosubstituted arabinose, a polypeptide having xylanase activity, a polypeptide having beta-xylosidase activity and optionally an alpha-xylosidase.

The present invention contemplates using the compositions of the present invention in saccharification, fermentation, or simultaneous saccharification and fermentation, to increase solubilization of hemicellulosic fibers to monomeric sugars, such as arabinose and xylose, in conventional and raw-starch hydrolysis (RSH) ethanol production processes. In other words, the processes of the present invention contemplate using any of the compositions or exemplary polypeptides described in this section.

A. Exemplary CE1 Polypeptides Having Ferulic Acid Esterase and/or Acetyl Xylan Esterase Activity

Aspects of the invention relate to compositions comprising a CE1 polypeptide having ferulic acid esterase and/or acetyl xylan esterase activity in combination with other enzymes to increase hemicellulosic fiber solubilization and production of monomeric arabinose and/or xylose. The present invention contemplates any CE1 polypeptide having ferulic acid esterase and/or acetyl xylan esterase activity that, when used in combination with polypeptides having arabinofuranosidase activity on di- and mono-substituted arabinose, a xylanase, a beta-xylosidase, and optionally an alpha-xylosidase, increases production of monomeric arabinose and/or xylose compared to compositions comprising the polypeptides having arabinofuranosidase activity on di- and mono-substituted arabinose, the xylanase, and the beta-xylosidase alone.

An exemplary CE1 polypeptide having ferulic acid esterase and/or acetyl xylan esterase activity has the amino acid sequence of SEQ ID NO: 3. In an embodiment, the CE1 polypeptide having ferulic acid esterase and/or acetyl xylan esterase activity has the amino acid sequence of SEQ ID NO: 3 with from 0 to 10 conservative amino acid substitutions and has ferulic acid esterase and/or acetyl xylan esterase activity. In an embodiment, the CE1 polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 3, and has ferulic acid esterase and/or acetyl xylan esterase activity. An exemplary CE1 polypeptide having ferulic acid esterase and/or acetyl xylan esterase activity has the amino acid sequence of SEQ ID NO: 6. In an embodiment, the CE1 polypeptide having ferulic acid esterase and/or acetyl xylan esterase activity has the amino acid sequence of SEQ ID NO: 6 with from 0 to 10 conservative amino acid substitutions and has ferulic acid esterase and/or acetyl xylan esterase activity. In an embodiment, the CE1 polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 6, and has ferulic acid esterase and/or acetyl xylan esterase activity. An exemplary CE1 polypeptide having ferulic acid esterase and/or acetyl xylan esterase activity has the amino acid sequence of SEQ ID NO: 9. In an embodiment, the CE1 polypeptide having ferulic acid esterase and/or acetyl xylan esterase activity has the amino acid sequence of SEQ ID NO: 9 with from 0 to 10 conservative amino acid substitutions and has ferulic acid esterase and/or acetyl xylan esterase activity. In an embodiment, the CE1 polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 9, and has ferulic acid esterase and/or acetyl xylan esterase activity. An exemplary CE1 polypeptide having ferulic acid esterase and/or acetyl xylan esterase activity has the amino acid sequence of SEQ ID NO: 12. In an embodiment, the CE1 polypeptide having ferulic acid esterase and/or acetyl xylan esterase activity has the amino acid sequence of SEQ ID NO: 12 with from 0 to 10 conservative amino acid substitutions and has ferulic acid esterase and/or acetyl xylan esterase activity. In an embodiment, the CE1 polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 12, and has ferulic acid esterase and/or acetyl xylan esterase activity. An exemplary CE1 polypeptide having ferulic acid esterase and/or acetyl xylan esterase activity has the amino acid sequence of SEQ ID NO: 15. In an embodiment, the CE1 polypeptide having ferulic acid esterase and/or acetyl xylan esterase activity has the amino acid sequence of SEQ ID NO: 15 with from 0 to 10 conservative amino acid substitutions and has ferulic acid esterase and/or acetyl xylan esterase activity. In an embodiment, the CE1 polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 15, and has ferulic acid esterase and/or acetyl xylan esterase activity.

The CE1 polypeptides having ferulic acid esterase and/or acetyl xylan esterase activity may be dosed in pre-saccharification, saccharification, and/or simultaneous saccharification and fermentation in a concentration of between 0.0001-1 mg EP (Enzyme Protein)/g DS, e.g., 0.0005-0.5 mg EP/g DS, such as 0.001-0.1 mg EP/g DS or 0.001-0.01 mg EP/g DS.

### B. Exemplary Polypeptides Having Arabinofuranosidase Activity on Disubstituted Arabinose

Aspects of the invention relate to compositions comprising arabinofuranosidases having activity on disubstituted arabinose in combination with other enzymes to increase hemicellulosic fiber solubilization and production of monomeric arabinose and/or xylose. The present invention contemplates any polypeptide having arabinofuranosidase activity on disubstituted arabinose that, when used in combination with a CE1 polypeptide having ferulic acid esterase and/or acetyl xylan esterase activity, a polypeptide having arabinofuranosidase on monosubstituted arabinose, a xylanase, a beta-xylosidase, and optionally an alpha-xylosidase, increases production of monomeric arabinose and/or xylose compared to compositions comprising the CE1 polypeptides, polypeptides having arabinofuranosidase activity on mono-substituted arabinose, the xylanase, the beta-xylosidase, and optionally the alpha-xylosidase alone.

In an embodiment, the polypeptide having arabinofuranosidase activity on disubstituted arabinose is a GH43 arabinofuranosidase. In an embodiment, the GH43 arabinofuranosidase is a GH43_36 arabinofuranosidase.

Exemplary GH43 arabinofuranosidases may be from the genus Humicola, Lasiodiplodia, or Poronia.

Exemplary GH43 arabinofuranosidases may be from the species Humicola insolens, Lasiodiplodia theobromae, or Poronia punctata.

An exemplary GH43 arabinofuranosidase has the amino acid sequence of SEQ ID NO: 16. In an embodiment, the GH43 arabinofuranosidase has the amino acid sequence of SEQ ID NO: 16 with from 0 to 10 conservative amino acid substitutions and has arabinofuranosidase activity. In an embodiment, the GH43 arabinofuranosidase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 16, and has arabinofuranosidase activity. An exemplary GH43 arabinofuranosidase has the amino acid sequence of SEQ ID NO: 17. In an embodiment, the GH43 arabinofuranosidase has the amino acid sequence of SEQ ID NO: 17 with from 0 to 10 conservative amino acid substitutions and has arabinofuranosidase activity. In an embodiment, the GH43 arabinofuranosidase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 17, and has arabinofuranosidase activity. An exemplary GH43 arabinofuranosidase has the amino acid sequence of SEQ ID NO: 18. In an embodiment, the GH43 arabinofuranosidase has the amino acid sequence of SEQ ID NO: 18 with from 0 to 10 conservative amino acid substitutions and has arabinofuranosidase activity. In an embodiment, the GH43 arabinofuranosidase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 18, and has arabinofuranosidase activity.

The polypeptides having arabinofuranosidase activity on disubstituted arabinose may be dosed in pre-saccharification, saccharification, and/or simultaneous saccharification and fermentation in a concentration of between 0.0001-1 mg EP (Enzyme Protein)/g DS, e.g., 0.0005-0.5 mg EP/g DS, such as 0.001-0.1 mg EP/g DS or 0.001-0.01 mg EP/g DS.

### C. Exemplary Polypeptides Having Arabinofuranosidase Activity on Monosubstituted Arabinose

Aspects of the invention relate to compositions comprising arabinofuranosidases having activity on monosubstituted arabinose in combination with other enzymes to increase hemicellulosic fiber solubilization and production of monomeric arabinose and/or xylose. The present invention contemplates any polypeptide having arabinofuranosidase activity on monosubstituted arabinose that, when used in combination with a CE1 polypeptide having ferulic acid esterase and/or acetyl xylan esterase activity, a polypeptide having arabinofuranosidase on disubstituted arabinose, a xylanase, a beta-xylosidase, and optionally an alpha-xylosidase, increases production of monomeric arabinose and/or xylose compared to compositions comprising the CE1 polypeptide having ferulic acid esterase and/or acetyl xylan esterase activity, the polypeptide having arabinofuranosidase activity on disubstituted arabinose, the xylanase, the beta-xylosidase, and optionally the alpha-xylosidase alone.

In an embodiment, the polypeptide having arabinofuranosidase activity on monosubstituted arabinose is a GH51 arabinofuranosidase. In an embodiment, the GH51 arabinofuranosidase is a GH51_6 arabinofuranosidase.

Exemplary GH51 arabinofuranosidases may be from the genus Meripulus, Lasiodiplodia, or Acidiella.

Exemplary GH51 arabinofuranosidases may be from the species Meripulus giganteus, Lasiodiplodia theobromae, or Acidiella bohemica.

An exemplary GH51 arabinofuranosidase has the amino acid sequence of SEQ ID NO: 19. In an embodiment, the GH51 arabinofuranosidase has the amino acid sequence of SEQ ID NO: 19 with from 0 to 10 conservative amino acid substitutions and has arabinofuranosidase activity. In an embodiment, the GH51 arabinofuranosidase has at least 60%, at least 65%, at least 70%, at least 75%, at least 80%, at least 85%, at least 90%, at least 95%, at least 96%, at least 97%, at least 98%, or at least 99% sequence to the amino acid sequence of SEQ ID NO: 19, and has arabinofuranosidase activity. An exemplary GH51 arabinofuranosidase has the amino acid sequence of SEQ ID NO: 20. In an embodiment, the GH43 arabinofuranosidase has the amino acid sequence of SEQ ID NO: 20 with from 0 to 10 conservative amino acid substitutions and has arabinofuranosidase activity. In an embodiment, the GH51 arabinofuranosidase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 20, which has arabinofuranosidase activity. An exemplary GH51 arabinofuranosidase has the amino acid sequence of SEQ ID NO: 21. In an embodiment, the GH51 arabinofuranosidase has the amino acid sequence of SEQ ID NO: 21 with from 0 to 10 conservative amino acid substitutions and has arabinofuranosidase activity. In an embodiment, the GH51 arabinofuranosidase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 21, and has arabinofuranosidase activity.

The polypeptides having arabinofuranosidase activity on monosubstituted arabinose may be dosed in pre-saccharification, saccharification, and/or simultaneous saccharification and fermentation in a concentration of between 0.0001-1 mg EP (Enzyme Protein)/g DS, e.g., 0.0005-0.5 mg EP/g DS, such as 0.001-0.1 mg EP/g DS or 0.001-0.01 mg EP/g DS.

### D. Exemplary Polypeptides Having Xylanase Activity

Aspects of the invention relate to compositions comprising polypeptides having xylanase activity in combination with other enzymes to increase hemicellulosic fiber solubilization and production of monomeric arabinose and/or xylose. The present invention contemplates any polypeptide having xylanase activity that, when used in combination with a CE1 polypeptide having ferulic acid esterase and/or acetyl xylan esterase activity, polypeptides having arabinofuranosidase activity on di- and mono-substituted arabinose, a beta-xylosidase, and optionally an alpha-xylosidase, increases production of monomeric arabinose and/or xylose compared to the compositions comprising CE1 polypeptide having ferulic acid esterase and/or acetyl xylan esterase activity, the polypeptides having arabinofuranosidase activity on di- and mono-substituted arabinose, the beta-xylosidase, and optionally alpha-xylosidase alone.

In an embodiment, the polypeptide having xylanase activity is a GH5_21 xylanase.

Exemplary GH_21 xylanases may be from the genus Bacteroides, Belliella, Chryseobacterium, or Sphingobacterium.

Exemplary GH_21 xylanases may be from the species Bacteroides cellulosilyticus CL02Y12C19, Belliella sp-64282, Chryseobacterium sp., Chryseobacterium oncorhynchi, or Sphingobacterium sp-64162.

Exemplary GH5_21 xylanases may be from bioreactor metagenome, Elephant dung metagenome, Xanthan alkaline community O, Xanthan alkaline community S, or Xanthan alkaline community T.

An exemplary GH5_21 xylanase has the amino acid sequence of SEQ ID NO: 22. In an embodiment, the GH5_21 xylanase has the amino acid sequence of SEQ ID NO: 22 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 22, and has xylanase activity. An exemplary GH5_21 xylanase has the amino acid sequence of SEQ ID NO: 23. In an embodiment, the GH5_21 xylanase has the amino acid sequence of SEQ ID NO: 23 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 23, and has xylanase activity. An exemplary GH5_21 xylanase has the amino acid sequence of SEQ ID NO: 24. In an embodiment, the GH5_21 xylanase has the amino acid sequence of SEQ ID NO: 24 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 24, and has xylanase activity. An exemplary GH5_21 xylanase has the amino acid sequence of SEQ ID NO: 25. In an embodiment, the GH5_21 xylanase has the amino acid sequence of SEQ ID NO: 25 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 25, and has xylanase activity. An exemplary GH5_21 xylanase has the amino acid sequence of SEQ ID NO: 26. In an embodiment, the GH5_21 xylanase has the amino acid sequence of SEQ ID NO: 26 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 26, and has xylanase activity. An exemplary GH5_21 xylanase has the amino acid sequence of SEQ ID NO: 27. In an embodiment, the GH5_21 xylanase has the amino acid sequence of SEQ ID NO: 27 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 27, and has xylanase activity. An exemplary GH5_21 xylanase has the amino acid sequence of SEQ ID NO: 28. In an embodiment, the GH5_21 xylanase has the amino acid sequence of SEQ ID NO: 28 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 28, and has xylanase activity. An exemplary GH5_21 xylanase has the amino acid sequence of SEQ ID NO: 29. In an embodiment, the GH5_21 xylanase has the amino acid sequence of SEQ ID NO: 29 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 29, and has xylanase activity. An exemplary GH521 xylanase has the amino acid sequence of SEQ ID NO: 30. In an embodiment, the GH5_21 xylanase has the amino acid sequence of SEQ ID NO: 30 with from 0 to 10 conservative amino acid substitutions and has xylanase activity. In an embodiment, the GH521 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 30, and has xylanase activity. An exemplary GH521 xylanase has the amino acid sequence of SEQ ID NO: 31. In an embodiment, the GH5_21 xylanase has the amino acid sequence of SEQ ID NO: 31 with from 0 to 10 conservative amino acid substitutions and has xylanase activity. In an embodiment, the GH521 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 31, and has xylanase activity. An exemplary GH521 xylanase has the amino acid sequence of SEQ ID NO: 32. In an embodiment, the GH5_21 xylanase has the amino acid sequence of SEQ ID NO: 32 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at and 98%, or at least 99% sequence identity to the amino acid sequence of SEQ ID NO: 32, and has xylanase activity. An exemplary GH5_21 xylanase has the amino acid sequence of SEQ ID NO: 33. In an embodiment, the GH5_21 xylanase has the amino acid sequence of SEQ ID NO: 33 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 33, and has xylanase activity. An exemplary GH5_21 xylanase has the amino acid sequence of SEQ ID NO: 34. In an embodiment, the GH5_21 xylanase has the amino acid sequence of SEQ ID NO: 34 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 34, and has xylanase activity. An exemplary GH5_21 xylanase has the amino acid sequence of SEQ ID NO: 35. In an embodiment, the GH5_21 xylanase has the amino acid sequence of SEQ ID NO: 35 with from 0 to 10 conservative amino acid substitutions and has xylanase activity. In an embodiment, the GH5_21 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 35, and has xylanase activity. An exemplary GH521 xylanase has the amino acid sequence of SEQ ID NO: 36. In an embodiment, the GH5_21 xylanase has the amino acid sequence of SEQ ID NO: 36 with from 0 to 10 conservative amino acid substitutions and has xylanase activity. In an embodiment, the GH521 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 36, and has xylanase activity.

In an embodiment, the polypeptide having xylanase activity is a GH535 xylanase.

Exemplary GH535 xylanases may be from the genus Bacillus, Cohnella, or Paenibacillus.

Exemplary GH535 xylanase may be from the species Bacillus hemiccellulosilyticus JCM 9152, Cohnella xylanilytica, Paenibacillus chitinolyticus, or Paenibacillus sp-62332.

Exemplary GH535 xylanases may be from compost metagenome.

An exemplary GH5_35 xylanase has the amino acid sequence of SEQ ID NO: 37. In an embodiment, the GH5_35 xylanase has the amino acid sequence of SEQ ID NO: 37 with from 0 to 10 conservative amino acid substitutions and has xylanase activity. In an embodiment, the GH535 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 37, and has xylanase activity. An exemplary GH5_35 xylanase has the amino acid sequence of SEQ ID NO: 38. In an embodiment, the GH5_35 xylanase has the amino acid sequence of SEQ ID NO: 38 with from 0 to 10 conservative amino acid substitutions and has xylanase activity. In an embodiment, the GH5_35 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 38, and has xylanase activity. An exemplary GH5_35 xylanase has the amino acid sequence of SEQ ID NO: 39. In an embodiment, the GH5_35 xylanase has the amino acid sequence of SEQ ID NO: 39 with from 0 to 10 conservative amino acid substitutions and has xylanase activity. In an embodiment, the GH5_35 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 39, and has xylanase activity. An exemplary GH5_35 xylanase has the amino acid sequence of SEQ ID NO: 40. In an embodiment, the GH5_35 xylanase has the amino acid sequence of SEQ ID NO: 40 with from 0 to 10 conservative amino acid substitutions and has xylanase activity. In an embodiment, the GH5_35 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 40, and has xylanase activity. An exemplary GH535 xylanase has the amino acid sequence of SEQ ID NO: 41. In an embodiment, the GH5_35 xylanase has the amino acid sequence of SEQ ID NO: 41 with from 0 to 10 conservative amino acid substitutions and has xylanase activity. In an embodiment, the GH535 xylanase has at least 60%, at least 65%, at least 70%, at least 75%, at least 80%, at least 85%, at least 90%, at least 95%, at least 96%, at least 97%, at least 98%, or at least 99% sequence to the amino acid sequence of SEQ ID NO: 41, and has xylanase activity. In an embodiment, the polypeptide having xylanase activity is a GH30_8 xylanase.

Exemplary GH30_8 xylanases may be from the genus Bacillus.

Exemplary GH30_8 xylanases may be from the species Bacillus sp-18423.

An exemplary GH30_8 xylanase has the amino acid sequence of SEQ ID NO: 42. In an embodiment, the GH30_8 xylanase has the amino acid sequence of SEQ ID NO: 42 with from 0 to 10 conservative amino acid substitutions and has xylanase activity. In an embodiment, the GH30_8 xylanase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 42, which has xylanase activity.

The polypeptides having xylanase activity may be dosed in pre-saccharification, saccharification, and/or simultaneous saccharification and fermentation in a concentration of between 0.0001-1 mg EP (Enzyme Protein)/g DS, e.g., 0.0005-0.5 mg EP/g DS, such as 0.001-0.1 mg EP/g DS or 0.001-0.01 mg EP/g DS.

### E. Exemplary Polypeptides Having Beta-Xylosidase Activity

Aspects of the invention relate to compositions comprising polypeptides having beta-xylosidase activity in combination with other enzymes to increase hemicellulosic fiber solubilization and production of monomeric arabinose and/or xylose. The present invention contemplates any polypeptide having beta-xylosidase activity that, when used in combination with a CE1 polypeptide having ferulic acid esterase and/or acetyl xylan esterase activity, polypeptides having arabinofuranosidase on di- and mono-substituted arabinose, a polypeptide having xylanase activity, a polypeptide having beta-xylosidase activity, and optionally a polypeptide having alpha-xylosidase activity, increases production of monomeric arabinose and/or xylose compared to compositions comprising the CE1 polypeptides, the polypeptides having arabinofuranosidase activity on di- and mono-substituted arabinose, the xylanase, and optionally the alpha-xylosidase alone.

In an embodiment, the beta-xylosidase is a GH3 beta-xylosidase.

Exemplary GH3 beta-xylosidases may be from the genus Aspergillus or Talaromyces.

Exemplary GH3 beta-xylosidases may be from the species Aspergillus fumigatus, Aspergillus nidulans, or Talaromyces emersonii.

An exemplary GH3 beta-xylosidase has the amino acid sequence of SEQ ID NO: 43. In an embodiment, the GH3 beta-xylosidase has the amino acid sequence of SEQ ID NO: 43 with from 0 to 10 conservative amino acid substitutions and has beta-xylosidase activity. In an embodiment, the GH3 beta-xylosidase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 43, and has beta-xylosidase activity. An exemplary GH3 beta-xylosidase has the amino acid sequence of SEQ ID NO: 44. In an embodiment, the GH3 beta-xylosidase has the amino acid sequence of SEQ ID NO: 44 with from 0 to 10 conservative amino acid substitutions and has beta-xylosidase activity. In an embodiment, the GH3 beta-xylosidase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 44, and has beta-xylosidase activity. An exemplary GH3 beta-xylosidase has the amino acid sequence of SEQ ID NO: 45. In an embodiment, the GH3 beta-xylosidase has the amino acid sequence of SEQ ID NO: 45 with from 0 to 10 conservative amino acid substitutions and has beta-xylosidase activity. In an embodiment, the GH3 beta-xylosidase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 45, which has beta-xylosidase activity.

The polypeptides having beta-xylosidase activity may be dosed in pre-saccharification, saccharification, and/or simultaneous saccharification and fermentation in a concentration of between 0.0001-1 mg EP (Enzyme Protein)/g DS, e.g., 0.0005-0.5 mg EP/g DS, such as 0.001-0.1 mg EP/g DS or 0.001-0.01 mg EP/g DS.

### F. Exemplary Polypeptides Having Alpha-Xylosidase Activity

Aspects of the invention relate to compositions comprising polypeptides having alpha-xylosidase activity in combination with other enzymes to increase hemicellulosic fiber solubilization and production of monomeric arabinose and/or xylose. The present invention contemplates any polypeptide having alpha-xylosidase activity that, when used in combination with a CE1 polypeptide having ferulic acid esterase and/or acetyl xylan esterase activity, polypeptides having arabinofuranosidase on di- and mono-substituted arabinose, a polypeptide having xylanase activity, polypeptide having beta-xylosidase activity, and optionally the polypeptide having alpha-xylosidase activity, increases production of monomeric arabinose and/or xylose compared to compositions comprising the CE1 polypeptides, the polypeptides having arabinofuranosidase activity on di- and mono-substituted arabinose, the xylanase, and the beta-xylosidase alone.

In an embodiment, the alpha-xylosidase comprises a GH31 alpha-xylosidase.

Exemplary GH31 alpha-xylosidases may be from the genus Herbinix.

Exemplary GH31 alpha-xylosidases may be from the species Herbinix hemicellulosilytica.

An exemplary GH31 alpha-xylosidase has the amino acid sequence of SEQ ID NO: 46. In an embodiment, the GH31 alpha-xylosidase has the amino acid sequence of SEQ ID NO: 46 with from 0 to 10 conservative amino acid substitutions and has beta-xylosidase activity. In an embodiment, the GH31 alpha-xylosidase has at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 46, which has alpha-xylosidase activity.

The polypeptides having alpha-xylosidase activity may be dosed in pre-saccharification, saccharification, and/or simultaneous saccharification and fermentation in a concentration of between 0.0001-1 mg EP (Enzyme Protein)/g DS, e.g., 0.0005-0.5 mg EP/g DS, such as 0.001-0.1 mg EP/g DS or 0.001-0.01 mg EP/g DS.

### G. Exemplary Fermenting Organisms

Aspects of the invention relate to the use of a fermenting organism for producing a fermentation product. Especially suitable fermenting organisms are able to ferment, i.e., convert, sugars, such as arabinose, glucose, maltose, and/or xylose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties). Other useful yeast strains are available from biological depositories such as the American Type Culture Collection (ATCC) or the Deutsche Sammlung von Mikroorganismen und Zellkulturen GmbH (DSMZ), such as, e.g., BY4741 (e.g., ATCC 201388); Y108-1 (ATCC PTA.10567) and NRRL YB-1952 (ARS Culture Collection). Still other S. cerevisiae strains suitable as host cells DBY746, [Alpha][Eta]22, S150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof.

As used herein, a “derivative” of strain is derived from a referenced strain, such as through mutagenesis, recombinant DNA technology, mating, cell fusion, or cytoduction between yeast strains. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species.

The fermenting organism may be Saccharomyces strain, e.g., Saccharomyces cerevisiae strain produced using the method described and concerned in U.S. Pat. No. 8,257,959-BB. In one embodiment, the recombinant cell is a derivative of a strain Saccharomyces cerevisiae CIBTS1260 (deposited under Accession No. NRRL Y-50973 at the Agricultural Research Service Culture Collection (NRRL), Illinois 61604 U.S.A.).

The fermenting organism may also be a derivative of Saccharomyces cerevisiae strain NMI V14/004037 (See, WO2015/143324 and WO2015/143317 each incorporated herein by reference), strain nos. V15/004035, V15/004036, and V15/004037 (See, WO 2016/153924 incorporated herein by reference), strain nos. V15/001459, V15/001460, V15/001461 (See, WO2016/138437 incorporated herein by reference), strain no. NRRL Y67342 (See, WO2018/098381 incorporated herein by reference), strain nos. NRRL Y67549 and NRRL Y67700 (See, WO 2019/161227 incorporated herein by reference), or any strain described in WO2017/087330 (incorporated herein by reference).

The fermenting organisms may comprise one or more heterologous polynucleotides encoding an alpha-amylase, glucoamylase, protease and/or cellulase. Examples of alpha-amylase, glucoamylase, protease and cellulases suitable for expression in the fermenting organism are known in the art (See, WO2021/231623 incorporated herein by reference),

The fermenting organism may be in the form of a composition comprising a fermenting organism and a naturally occurring and/or a non-naturally occurring component. The fermenting organism may be in any viable form, including crumbled, dry, including active dry and instant, compressed, cream (liquid) form etc. In one embodiment, the fermenting organism (e.g., a Saccharomyces cerevisiae yeast strain) is dry yeast, such as active dry yeast or instant yeast. In one embodiment, the fermenting organism is crumbled yeast. In one embodiment, the fermenting organism is a compressed yeast. In one embodiment, the fermenting organism is cream yeast.

In one embodiment is a composition comprising a fermenting organism described herein (e.g., a Saccharomyces cerevisiae yeast strain), and one or more of the components selected from the group consisting of: surfactants, emulsifiers, gums, swelling agent, and antioxidants and other processing aids.

The compositions described herein may comprise a fermenting organism described herein (e.g., a Saccharomyces cerevisiae yeast strain) and any suitable surfactants. In one embodiment, the surfactant(s) is/are an anionic surfactant, cationic surfactant, and/or nonionic surfactant.

The compositions described herein may comprise a fermenting organism described herein (e.g., a Saccharomyces cerevisiae yeast strain) and any suitable emulsifier. In one embodiment, the emulsifier is a fatty-acid ester of sorbitan. In one embodiment, the emulsifier is selected from the group of sorbitan monostearate (SMS), citric acid esters of monodiglycerides, polyglycerolester, fatty acid esters of propylene glycol.

In one embodiment, the composition comprises a fermenting organism described herein (e.g., a Saccharomyces cerevisiae yeast strain), and Olindronal SMS, Olindronal SK, or Olindronal SPL including composition concerned in European Patent No. 1,724,336 (hereby incorporated by reference). These products are commercially available from Bussetti, Austria, for active dry yeast.

The compositions described herein may comprise a fermenting organism described herein (e.g., a Saccharomyces cerevisiae yeast strain) and any suitable gum. In one embodiment, the gum is selected from the group of carob, guar, tragacanth, arabic, xanthan and acacia gum, in particular for cream, compressed and dry yeast.

The compositions described herein may comprise a fermenting organism described herein (e.g., a Saccharomyces cerevisiae yeast strain) and any suitable swelling agent. In one embodiment, the swelling agent is methyl cellulose or carboxymethyl cellulose.

The compositions described herein may comprise a fermenting organism described herein (e.g., a Saccharomyces cerevisiae yeast strain) and any suitable anti-oxidant. In one embodiment, the antioxidant is butylated hydroxyanisol (BHA) and/or butylated hydroxytoluene (BHT), or ascorbic acid (vitamin C), particular for active dry yeast.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Process for Producing a Fermentation Product from a Gelatinized Starch-Containing Material

An aspect of the invention relates to a process for producing a fermentation product, (e.g., fuel ethanol), from a gelatinized starch-containing material, wherein a composition comprising a CE1 polypeptide having ferulic acid esterase and/or acetyl xylan esterase activity, or CE1 polypeptide having ferulic acid esterase and/or acetyl xylan esterase activity, is present or added during saccharification and/or fermentation.

In an embodiment, a process for producing a fermentation product from a starch-containing material, comprises the steps of:
(a) liquefying a starch-containing material at a temperature above the initial gelatinization temperature of the starch using a thermostable alpha-amylase to produce a dextrin;
(b) saccharifying the dextrin using a glucoamylase to produce a fermentable sugar; and
(c) fermenting the sugar using a fermenting organism to produce the fermentation product;
wherein a CE1 polypeptide having ferulic acid esterase and/or acetyl xylan esterase activity or a composition comprising a CE1 polypeptide having ferulic acid esterase and/or acetyl xylan esterase activity is present or added during saccharifying step (b) and/or fermenting step (c)

The present invention contemplates using any of the CE1 polypeptides or compositions described herein in the process for producing the fermentation product. In an embodiment, the composition used in step (b) and/or step (c) includes a polypeptide having arabinofuranosidase activity on disubstituted arabinose. In an embodiment, the polypeptide having arabinofuranosidase activity on disubstituted arabinose is a GH43 arabinofuranosidase. In an embodiment, the GH43 arabinofuranosidase is a GH43_36 arabinofuranosidase. In an embodiment, the composition used in step (b) and/or step (c) includes a polypeptide having arabinofuranosidase activity on mono-substituted arabinose. In embodiment, the polypeptide having arabinofuranosidase activity on mono-substituted arabinose is a GH51 arabinofuranosidase. In an embodiment, the GH51 arabinofuranosidase is a GH51_6 arabinofuranosidase. In an embodiment, the composition used in step (b) and/or step (c) includes a polypeptide having xylanase activity. In an embodiment, the polypeptide having xylanase activity is a GH5 family xylanase. In an embodiment, the GH5 family xylanase is a GH5_21 xylanase. In an embodiment, the GH5 family xylanase is a GH5_35 xylanase. In an embodiment, the polypeptide having xylanase activity is a GH30_8 xylanase. In an embodiment, the composition used in step (b) and/or step (c) includes a beta-xylosidase. In an embodiment, the beta-xylosidase is a GH3 beta-xylosidase. In an embodiment, the composition used in step (b) and/or step (c) includes an alpha-xylosidase. In an embodiment, the alpha-xylosidase is a GH31 alpha-xylosidase.

The present invention contemplates using any of the exemplary CE1 polypeptides, exemplary polypeptides having arabinofuranosidase activity on di- and mono-substituted arabinose, exemplary polypeptides having xylanse activity, exemplary polypeptides having beta-xylosidase activity, and exemplary polypeptides having alpha-xylosidase activity described above in the compositions and processes of using the compositions of the invention, including in the following exemplary compostions used in the process for producing a fermentation product.

An exemplary composition used in step (b) and/or step (c) comprises a CE1 polypeptide having ferulic acid esterase and/or acetyl xylan esterase activity, a polypeptide having arabinofuranosidase activity on disubstituted arabinose, a polypeptide having arabinofuranosidase activity on monosubstituted arabinofuranosidase, a polypeptide having xylanase activity, and a polypeptide having beta-xylosidase activity.

An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a polypeptide having xylanase activity, and a polypeptide having beta-xylosidase activity. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5 xylanase, and a polypeptide having beta-xylosidase activity. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5 xylanase, and a GH3 beta-xylosidase. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5_21 xylanase, and a GH3 beta-xylosidase. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5_35 xylanase, and a GH3 beta-xylosidase. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH30_8 xylanase, and a GH3 beta-xylosidase.

An exemplary composition used in step (b) and/or step (c) comprises a CE1 polypeptide having ferulic acid esterase and/or acetyl xylan esterase activity, a GH43_36 arabinofuranosidase, a GH51_6 arabinofuranosidase, a polypeptide having xylanase activity, and a polypeptide having beta-xylosidase activity. An exemplary composition used in step (b) and/or step (c) comprises a CE1 polypeptide having ferulic acid esterase and/or acetyl xylan esterase activity, a GH43_36 arabinofuranosidase, a GH51_6 arabinofuranosidase, a GH5 xylanase, and a polypeptide having beta-xylosidase activity. An exemplary composition used in step (b) and/or step (c) comprises a CE1 polypeptide having ferulic acid esterase and/or acetyl xylan esterase activity, a GH43_36 arabinofuranosidase, a GH51_6 arabinofuranosidase, a GH5 xylanase, and a GH3 beta-xylosidase. An exemplary composition used in step (b) and/or step (c) comprises a CE1 polypeptide having ferulic acid esterase and/or acetyl xylan esterase activity, a GH43_36 arabinofuranosidase, a GH51_6 arabinofuranosidase, a GH5_21 xylanase, and a GH3 beta-xylosidase. An exemplary composition used in step (b) and/or step (c) comprises a CE1 polypeptide having ferulic acid esterase and/or acetyl xylan esterase activity, a GH43_36 arabinofuranosidase, a GH51_6 arabinofuranosidase, a GH5_35 xylanase, and a GH3 beta-xylosidase. An exemplary composition used in step (b) and/or step (c) comprises a CE1 polypeptide having ferulic acid esterase and/or acetyl xylan esterase activity, a GH43_36 arabinofuranosidase, a GH51_6 arabinofuranosidase, a GH30_8 xylanase, and a GH3 beta-xylosidase.

An exemplary composition used in step (b) and/or step (c) comprises a CE1 polypeptide having ferulic acid esterase and/or acetyl xylan esterase activity, a polypeptide having arabinofuranosidase activity on disubstituted arabinose, a polypeptide having arabinofuranosidase activity on monosubstituted arabinofuranosidase, a polypeptide having xylanase activity, a polypeptide having beta-xylosidase activity, and a polypeptide having alpha-xylosidase activity.

An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a polypeptide having xylanase activity, a polypeptide having beta-xylosidase activity, and a polypeptide having alpha-xylosidase activity. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5 xylanase, a polypeptide having beta-xylosidase activity, and a polypeptide having alpha-xylosidase activity. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5 xylanase, a GH3 beta-xylosidase, and a polypeptide having alpha-xylosidase activity. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5_21 xylanase, a GH3 beta-xylosidase, and a polypeptide having alpha-xylosidase activity. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5_35 xylanase, a GH3 beta-xylosidase, and a polypeptide having alpha-xylosidase activity. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH30_8 xylanase, and a GH3 beta-xylosidase, and a polypeptide having alpha-xylosidase activity. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5 xylanase, a GH3 beta-xylosidase, and a GH31 alpha-xylosidase. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5_21 xylanase, a GH3 beta-xylosidase, and a GH31 alpha-xylosidase. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH5_35 xylanase, a GH3 beta-xylosidase, and a GH31 alpha-xylosidase. An exemplary composition used in step (b) and/or step (c) comprises a CE1 polypeptide having ferulic acid esterase and/or acetyl xylan esterase activity, a GH43 arabinofuranosidase, a GH51 arabinofuranosidase, a GH30_8 xylanase, and a GH3 beta-xylosidase, and a GH31 alpha-xylosidase.

In an embodiment, the composition is added during saccharifying step (b). In an embodiment, the composition is added during fermenting step (c). In an embodiment, steps (b) and (c) are performed simultaneously in a simultaneous saccharification and fermentation (SSF). In an embodiment, the composition is added during SSF.

In an embodiment, a thermostable glucoamylase is added during liquefying step (a). In an embodiment, a thermostable endoglucanase is added during liquefying step (a). In an embodiment, a thermostable lipase is added during liquefying step (a). In an embodiment, a thermostable phytase is added during liquefying step (a). In an embodiment, a thermostable protease is added during liquefying step (a). In an embodiment, a thermostable pullulanase is added during liquefying step (a). In an embodiment, a thermostable xylanase is added during liquefying step (a). In a preferred embodiment, a thermostable alpha-amylase and a thermostable protease are added during liquefying step (a). In an embodiment, a thermostable alpha-amylase and a thermostable xylanase are added during liquefying step (a). In a preferred embodiment, a thermostable alpha-amylase, a thermostable protease and a thermostable xylanase are added during liquefying step (a).

In an embodiment, an alpha-amylase is added during step (b) and/or step (c). In an embodiment, an alpha-glucosidase is added during step (b) and/or step (c). In an embodiment, a beta-amylase is added during step (b) and/or step (c). In an embodiment, a beta-glucanase is added during step (b) and/or step (c). In an embodiment, a beta-glucosidase is added during step (b) and/or step (c). In an embodiment, a cellobiohydrolase is added during step (b) and/or step (c). In an embodiment, an endoglucanase is added during step (b) and/or step (c). In an embodiment a lipase is added during step (b) and/or step (c). In an embodiment, a lytic polysaccharide monooxygenase (LPMO) is added during step (b) and/or step (c). In an embodiment, a maltogenic alpha-amylase is added during step (b) and/or step (c). In an embodiment, a pectinase is added during step (b) and/or step (c). In an embodiment, a peroxidase is added during step (b) and/or step (c). In an embodiment, a phytase is added during step (b) and/or step (c). In an embodiment, a protease is added during step (b) and/or step (c). In an embodiment, a trehalase is added during step (b) and/or step (c).

In an embodiment, the fermenting organism is yeast. In an embodiment, the yeast expresses an alpha-amylase in situ during step (b) and/or step (c). In an embodiment, the yeast expresses a glucoamylase in situ during step (b) and/or step (c).

### Process Parameters

The parameters for processes for producing fermentation products, such as the production of ethanol from starch-containing material (e.g., corn) are well known in the art. See, e.g., WO 2006/086792, WO 2013/082486, WO 2012/088303, WO 2013/055676, WO 2014/209789, WO 2014/209800, WO 2015/035914, WO 2017/112540, WO 2020/014407, WO 2021/126966 (each of which is incorporated herein by reference).

### Starch-Containing Material

Any suitable starch-containing starting material may be used. The material is selected based on the desired fermentation product. Examples of starch-containing materials, include without limitation, barley, beans, cassava, cereals, corn, milo, peas, potatoes, rice, rye, sago, sorghum, sweet potatoes, tapioca, wheat, and whole grains, or any mixture thereof. The starch-containing material may also be a waxy or non-waxy type of corn and barley. Commonly used commercial starch-containing materials include corn, milo and/or wheat.

### Starch-Containing Material Particle Size Reduction

Prior to liquefying step (a), the particle size of the starch-containing material may be reduced, for example by dry milling.

### Slurry

Prior to liquefying step (a), a slurry comprising the starch-containing material (e.g., preferably milled) and water may be formed. Alpha-amylase and optionally protease may be added to the slurry. The slurry may be heated to between to above the initial gelatinization temperature of the starch-containing material to begin gelatinization of the starch.

### Jet Cook

The slurry may optionally be jet-cooked to further gelatinize the starch in the slurry before adding alpha-amylase during liquefying step (a). Jet cooking can be performed at temperatures ranging from 100° C. to 120° C. for up to at least 15 minutes.

### Liquefaction Temperature

The temperature used during liquefying step (a) may range from 70° C. to 110° C., such as from 75° C. to 105° C., from 80° C. to 100° C., from 85° C. to 95° C., or from 88° C. to 92° C. Preferably, the temperature is at least 70° C., at least 80° C., at least 85° C., at least 88° C., or at least 90° C.

### Liquefaction pH

The pH used during liquefying step (a) may range from 4 to 6, from 4.5 to 5.5, or from 4.8 to 5.2. Preferably, the pH is at least 4.5, at least 4.6, at least 4.7, at least 4.8, at least 4.9, at least 5.0, or at least 5.1.

### Liquefaction Time

The time for performing liquefying step (a) may range from 30 minutes to 5 hours, from 1 hour to 3 hours, or 90 minutes to 150 minutes. Preferably, the time is at least 30 minutes, at least about 45 minutes, at least about 60 minutes, at least about 90 minutes, or at least about 2 hours.

### Liquefaction Enzymes

The present invention contemplates the use of thermostable enzymes during liquefying step (a). It is well known in the art to use various thermostable enzymes during liquefying step (a), including, for example, thermostable alpha-amylases, thermostable glucoamylases, thermostable endoglucanases, thermostable lipases, thermostable phytase, thermostable proteases, thermostable pullulanases, and/or thermostable xylanases. The present invention contemplates the use of any thermostable enzyme in liquefying step (a). Guidance for determining the denaturation temperature of a candidate thermostable enzyme for use in liquefying step (a) is provided in the Materials & Methods section below. The published patent applications listed below describe activity assays for determining whether a candidate thermostable enzyme contemplated for use in liquefying step (a) will be deactivated at a temperature contemplated for liquefying step (a).

Examples of suitable thermostable alpha-amylases and guidance for using them in liquefying step (a) include, without limitation, the alpha-amylases described in WO94/18314, WO94/02597, WO 96/23873, WO 96/23874, WO 96/39528, WO 97/41213, WO 97/43424, WO 99/19467, WO 00/60059, WO 2002/010355, WO 2002/092797, WO 2009/149130, WO 2009/61378, WO 2009/061379, WO 2009/061380, WO 2009/061381, WO 2009/098229, WO 2009/100102, WO 2010/115021, WO2010/115028, WO 2010/036515, WO 2011/082425, WO 2013/096305, WO 2013/184577, WO 2014/007921, WO 2014/164777, WO 2014/164800, WO 2014/164834, WO 2019/113413, WO 2019/113415, WO 2019/197318 (each of which is incorporated herein by reference).

Examples of suitable thermostable glucoamylases include, without limitation, the glucoamylases described in WO 2011/127802, WO 2013/036526, WO 2013/053801, WO 2018/164737, WO 2020/010101, and WO 2022/090564 (each of which is incorporated herein by reference).

Examples of suitable thermostable endoglucanases include, without limitation, the endoglucanases described in WO 2015/035914 (which is incorporated herein by reference)

Examples of suitable thermostable lipases include, without limitation, the lipases described in WO 2017/112542 and WO 2020/014407 (which are both incorporated herein by reference).

Examples of suitable thermostable phytases include, without limitation, the phytases described in WO 1996/28567, WO 1997/33976, WO 1997/38096, WO 1997/48812, WO 1998/05785, WO 1998/06856, WO 1998/13480, WO 1998/20139, WO 1998/028408, WO 1999/48330, WO 1999/49022, WO 2003/066847, WO 2004/085638, WO 2006/037327, WO 2006/037328, WO 2006/038062, WO 2006/063588, WO 2007/112739, WO 2008/092901, WO 2008/116878, WO 2009/129489, and WO 2010/034835 (each of which is incorporated by reference). Commercially available phytase containing products include BIO-FEED PHYTASE™ PHYTASE NOVO™ CT or L, LIQMAX or RONOZYME™ NP, RONOZYME® HIPHOS, RONOZYME® P5000 (CT), NATUPHOS™ NG 5000.

Examples of suitable thermostable proteases include, without limitation, the proteases described in WO 1992/02614, WO 98/56926, WO 2001/151620, WO 2003/048353, WO 2006/086792, WO 2010/008841, WO 2011/076123, WO 2011/087836, WO 2012/088303, WO 2013/082486, WO 2014/209789, WO 2014/209800, WO 2018/098124, WO2018/118815 A1, and WO2018/169780A1 (each of which is incorporated herein by reference).

Suitable commercially available protease containing products include AVANTEC AMP®, FORTIVA REVO®, FORTIVA HEMI®.

Examples of suitable thermostable pullulanases include, without limitation, the pullulanases described in WO 2015/007639, WO 2015/110473, WO 2016/087327, WO 2017/014974, and WO 2020/187883 (each of which is incorporated herein by reference in its entirety). Suitable commercially available pullulanase products include PROMOZYME 400L, PROMOZYME™ D2 (Novozymes A/S, Denmark), OPTIMAX L-300 (Genencor Int., USA), and AMANO 8 (Amano, Japan).

Examples of suitable thermostable xylanases include, without limitation, the xylanases described in WO 2017/112540 and WO 2021/126966 (each of which is incorporated herein by reference). Suitable commercially available thermostable xylanase containing products include FORTIVA HEMI®.

The enzyme(s) described above are to be used in effective amounts in the processes of the present invention. Guidance for determining effective amounts of enzymes to be used in liquefying step (a) can be found in the published patent applications cited for each of the different thermostable liquefaction enzymes, along with guidance for performing activity assays for determining the activity of those enzymes.

### Saccharification Temperature

Saccharification may be performed at temperatures ranging from 20° C. to 75° C., from 30° C. to 70° C., or from 40° C. to 65° C. Preferably, the saccharification temperature is at least about 50° C., at least about 55° C., or at least about 60° C.

### Saccharification pH

Saccharification may occur at a ph ranging from 4 to 5. Preferably, the pH is about 4.5.

### Saccharification Time

Saccharification may last from about 24 hours to about 72 hours.

### Fermentation Time

Fermentation may last from 6 to 120 hours, from 24 hours to 96 hours, or from 35 hours to 60 hours.

### Simultaneous Saccharification and Fermentation

SSF may be performed at a temperature from 25° C. to 40° C., from 28° C. to 35° C., or from 30° C. to ° C., at a pH from 3.5 to 5 or from 3.8 to 4.3, for 24 to 96 hours, 36 to 72 hours, or from 48 to 60 hours. Preferably, SSF is performed at about 32° C., at a pH from 3.8 to 4.5 for from 48 to 60 hours.

Saccharification and/or Fermentation Enzymes

The present invention contemplates the use of enzymes during saccharifying step (b) and/or fermenting step (c). It is well known in the art to use various enzymes during saccharifying step (b) and/or fermenting step (c), including, for example, alpha-amylases, alpha-glucosidases, beta-amylases, beta-glucanases, beta-glucosidases, cellobiohydrolases, endoglucanases, glucoamylases, lipases, lytic polysaccharide monooxygenases (LPMOs), maltogenic alpha-amylases, pectinases, peroxidases, phytases, proteases, and trehalases.

The enzymes used in saccharifying step (b) and/or fermenting step (c) may be added exogenously as mono-components or formulated as compositions comprising the enzymes. The enzymes used in saccharifying step (b) and/or fermenting step (c) may also be added via in situ expression from the fermenting organism (e.g., yeast).

Examples of suitable alpha-amylases include, without limitation, the alpha-amylases described in WO 2004/055178, WO 2006/069290, WO 2013/006756, WO 2013/034106, WO 2013/044867, WO 2021/163011, and WO 2021/163030 (each of which is incorporated herein by reference).

Examples of suitable glucoamylases include, without limitation, the glucoamylases described in WO 1984/02921, WO 1992/00381, WO 1999/28448, WO 2000/04136, WO 2001/04273, WO 2006/069289, WO 2011/066560, WO 2011/066576, WO 2011/068803, WO 2011/127802, WO 2012/064351, WO 2013/036526, WO 2013/053801, WO 2014/039773, WO 2014/177541, WO 2014/177546, WO 2016/062875, WO 2017/066255, and WO 2018/191215 (each of which is incorporated herein by reference.

Examples of suitable compositions comprising alpha-amylases and glucoamylases include, without limitation, the compostions described in WO 2006/069290, WO 2009/052101, WO 2011/068803, and WO 2013/006756 (each of which is incorporated by reference herein). Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL, SPIRIZYME ACHIEVE and AMG™ E (from Novozymes A/S); OPTIDEX™ 300, GC480, GC417 (from DuPont-Genencor); AMIGASE™ and AMIGASE™ PLUS (from DSM); G-ZYME™ G900, G-ZYME™ and G990 ZR (from DuPont-Genencor).

Examples of suitable beta-glucanases include, without limitation, the beta-glucanases described in WO 2021/055395 (which is incorporated herein by reference).

Examples of suitable beta-glucosidases include, without limitation, the beta-glucosidases described in WO 2005/047499, WO 2013/148993, WO 2014/085439 and WO 2012/044915 (each of which is incorporated herein by reference).

Examples of suitable cellobiohydrolases include, without limitation, the cellobiohydrolases described in WO 2013/148993, WO 2014/085439, WO 2014/138672, and WO 2016/040265 (each of which is incorporated herein by reference).

Examples of suitable endoglucanases include, without limitation, the endoglucanases described in WO 2013/148993 and WO 2014/085439 (both of which are incorporated herein by reference).

Examples of suitable maltogenic alpha-amylases are described in U.S. Pat. Nos. 4,598,048, 4,604,355 and 6,162,628, which are hereby incorporated by reference.

Examples of suitable lipases include, without limitation, the lipases described in WO 2017/112533, WO 2017/112539, and WO 2020/076697 (each of which is incorporated herein by reference).

Examples of suitable LPMOs include, without limitation, the LPMOs described in WO 2013/148993, WO 2014/085439, and WO 2019/083831 (each of which is incorporated herein by reference).

Examples of suitable phytases include, without limitation, the phytases described in WO 2001/62947 (which is incorporated herein by reference).

Examples of suitable pectinases include, without limitation, the pectinases described in WO 2022/173694 (which is incorporated herein by reference).

Examples of suitable peroxidases include, without limitation, the peroxidases described in WO 2019/231944 (which is incorporated herein by reference).

Examples of suitable proteases include, without limitation, the proteases described in WO 2017/050291, WO 2017/148389, WO 2018/015303, and WO 2018/015304 (each of which is incorporated herein by reference).

Examples of suitable trehalases include, without limitation, the trehalases described in WO 2016/205127, WO 2019/005755, WO 2019/030165, and WO 2020/023411 (each of which is incorporated herein by reference).

Process for Producing a Fermentation Product from Ungelatinized Starch-Containing Material

An aspect of the invention relates to a process for producing a fermentation product from an ungelatinized starch-containing material (i.e., granularized starch—often referred to as a “raw starch hydrolysis” process), wherein a composition comprising a CE1 polypeptide having ferulic acid esterase and/or acetyl xylan esterase activity, or CE1 polypeptide having ferulic acid esterase and/or acetyl xylan esterase activity, is present or added during saccharification and/or fermentation.

In an embodiment, a process for producing a fermentation product from an ungelatinized starch-containing material comprises the following steps:
(a) saccharifying a starch-containing material at a temperature below the initial gelatinization temperature of the starch using an alpha-amylase and a glucoamylase to produce a fermentable sugar; and
(b) fermenting the sugar using a fermentation organism to produce a fermentation product;
wherein a CE1 polypeptide having ferulic acid esterase and/or acetyl xylan esterase activity or a composition comprising a CE1 polypeptide having ferulic acid esterase and/or acetyl xylan esterase activity is present or added during saccharifying step (a) and/or fermenting step (b)

The present invention contemplates any of the CE1 polypeptides or compositions described herein for use in the process for producing the fermentation product. In an embodiment, the composition used in step (a) and/or step (b) includes a polypeptide having arabinofuranosidase activity on disubstituted arabinose. In an embodiment, the polypeptide having arabinofuranosidase activity on disubstituted arabinose is a GH43 arabinofuranosidase. In an embodiment, the GH43 arabinofuranosidase is a GH43_36 arabinofuranosidase. In an embodiment, the composition used in step (a) and/or step (b) includes a polypeptide having arabinofuranosidase activity on mono-substituted arabinose. In embodiment, the polypeptide having arabinofuranosidase activity on mono-substituted arabinose is a GH51 arabinofuranosidase. In an embodiment, the GH51 arabinofuranosidase is a GH51_6 arabinofuranosidase. In an embodiment, the composition used in step (a) and/or step (b) includes a polypeptide having xylanase activity. In an embodiment, the polypeptide having xylanase activity is a GH5 family xylanase. In an embodiment, the GH5 family xylanase is a GH5_21 xylanase. In an embodiment, the GH5 family xylanase is a GH5_35 xylanase. In an embodiment, the polypeptide having xylanase activity is a GH30_8 xylanase. In an embodiment, the composition used in step (a) and/or step (b) includes a beta-xylosidase. In an embodiment, the beta-xylosidase is a GH3 beta-xylosidase. In an embodiment, the composition used in step (a) and/or step (b) includes an alpha-xylosidase. In an embodiment, the alpha-xylosidase is a GH31 alpha-xylosidase.

The present invention contemplates using any of the exemplary CE1 polypeptides, exemplary polypeptides having arabinofuranosidase activity on di- and mono-substituted arabinose, exemplary polypeptides having xylanse activity, exemplary polypeptides having beta-xylosidase activity, and exemplary polypeptides having alpha-xylosidase activity described above in the compositions and processes of using the compositions of the invention, including in the following exemplary compostions used in the process for producing a fermentation product.

An exemplary composition used in step (a) and/or step (b) comprises a CE1 polypeptide having ferulic acid esterase and/or acetyl xylan esterase activity, a polypeptide having arabinofuranosidase activity on disubstituted arabinose, a polypeptide having arabinofuranosidase activity on monosubstituted arabinofuranosidase, a polypeptide having xylanase activity, and a polypeptide having beta-xylosidase activity.

An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a polypeptide having xylanase activity, and a polypeptide having beta-xylosidase activity. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5 xylanase, and a polypeptide having beta-xylosidase activity. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5 xylanase, and a GH3 beta-xylosidase. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5_21 xylanase, and a GH3 beta-xylosidase. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5_35 xylanase, and a GH3 beta-xylosidase. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH30_8 xylanase, and a GH3 beta-xylosidase.

An exemplary composition used in step (a) and/or step (b) comprises a CE1 polypeptide having ferulic acid esterase and/or acetyl xylan esterase activity, a GH43_36 arabinofuranosidase, a GH51_6 arabinofuranosidase, a polypeptide having xylanase activity, and a polypeptide having beta-xylosidase activity. An exemplary composition used in step (a) and/or step (b) comprises a CE1 polypeptide having ferulic acid esterase and/or acetyl xylan esterase activity, a GH43_36 arabinofuranosidase, a GH51_6 arabinofuranosidase, a GH5 xylanase, and a polypeptide having beta-xylosidase activity. An exemplary composition used in step (a) and/or step (b) comprises a CE1 polypeptide having ferulic acid esterase and/or acetyl xylan esterase activity, a GH43_36 arabinofuranosidase, a GH51_6 arabinofuranosidase, a GH5 xylanase, and a GH3 beta-xylosidase. An exemplary composition used in step (a) and/or step (b) comprises a CE1 polypeptide having ferulic acid esterase and/or acetyl xylan esterase activity, a GH43_36 arabinofuranosidase, a GH51_6 arabinofuranosidase, a GH5_21 xylanase, and a GH3 beta-xylosidase. An exemplary composition used in step (a) and/or step (b) comprises a CE1 polypeptide having ferulic acid esterase and/or acetyl xylan esterase activity, a GH43_36 arabinofuranosidase, a GH51_6 arabinofuranosidase, a GH5_35 xylanase, and a GH3 beta-xylosidase. An exemplary composition used in step (a) and/or step (b) comprises a CE1 polypeptide having ferulic acid esterase and/or acetyl xylan esterase activity, a GH43_36 arabinofuranosidase, a GH51_6 arabinofuranosidase, a GH30_8 xylanase, and a GH3 beta-xylosidase.

An exemplary composition used in step (a) and/or step (b) comprises a CE1 polypeptide having ferulic acid esterase and/or acetyl xylan esterase activity, a polypeptide having arabinofuranosidase activity on disubstituted arabinose, a polypeptide having arabinofuranosidase activity on monosubstituted arabinofuranosidase, a polypeptide having xylanase activity, a polypeptide having beta-xylosidase activity, and a polypeptide having alpha-xylosidase activity.

An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a polypeptide having xylanase activity, a polypeptide having beta-xylosidase activity, and a polypeptide having alpha-xylosidase activity. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5 xylanase, a polypeptide having beta-xylosidase activity, and a polypeptide having alpha-xylosidase activity. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5 xylanase, a GH3 beta-xylosidase, and a polypeptide having alpha-xylosidase activity. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5_21 xylanase, a GH3 beta-xylosidase, and a polypeptide having alpha-xylosidase activity. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5_35 xylanase, a GH3 beta-xylosidase, and a polypeptide having alpha-xylosidase activity. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH30_8 xylanase, and a GH3 beta-xylosidase, and a polypeptide having alpha-xylosidase activity. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5 xylanase, a GH3 beta-xylosidase, and a GH31 alpha-xylosidase. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5_21 xylanase, a GH3 beta-xylosidase, and a GH31 alpha-xylosidase. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH5_35 xylanase, a GH3 beta-xylosidase, and a GH31 alpha-xylosidase. An exemplary composition used in step (a) and/or step (b) comprises a CE1 polypeptide having ferulic acid esterase and/or acetyl xylan esterase activity, a GH43 arabinofuranosidase, a GH51 arabinofuranosidase, a GH30_8 xylanase, and a GH3 beta-xylosidase, and a GH31 alpha-xylosidase.

In an embodiment, the composition is added during saccharifying step (b). In an embodiment, the composition is added during fermenting step (c). In an embodiment, steps (b) and (c) are performed simultaneously in a simultaneous saccharification and fermentation (SSF). In an embodiment, the composition is added during SSF.

Raw starch hydrolysis (RSH) processes are well-known in the art. The skilled artisan will appreciate that, except for the process parameters relating to liquefying step (a) which is not done in a RSH process, the process parameters described in Section II above are applicable to the process described in this section, including selection of the starch-containing material, reducing the grain particle size, saccharification temperature, time and pH, conditions for simultaneous saccharification and fermentation, and saccharification enzymes. The process parameters for an exemplary raw-starch hydrolysis process are described in further detail in WO 2004/106533 (which is incorporated herein by reference).

Examples of alpha-amylases that are preferably used in step (a) and/or step (b) include, without limitation, the alpha-amylases described in WO 2004/055178, WO 2005/003311, WO 2006/069290, WO 2013/006756, WO 2013/034106, WO 2021/163015, and WO 2021/163036 (each of which is incorporated by reference herein).

Examples of glucoamylases that are preferably used in step (a) and/or step (b) include, without limitation, WO 1999/28448, WO 2005/045018, WO2005/069840, WO 2006/069289 (each of which is incorporated by reference herein).

Examples of compositions comprising alpha-amylases and glucoamylase that are preferably used in step (a) and/or step (b) include, without limitation, the compositions described in WO 2015/031477 (which is incorporated by reference herein).

### Backend or Downstream Processing

### A. Recovery of the Fermentation Product and Production of Whole Stillage

Subsequent to fermentation or SSF, the fermentation product may be separated from the fermentation medium. The fermentation product, e.g., ethanol, can optionally be recovered from the fermentation medium using any method known in the art including, but not limited to, chromatography, electrophoretic procedures, differential solubility, distillation, or extraction. For example, alcohol is separated from the fermented starch-containing material and purified by conventional methods of distillation.

Thus, in one embodiment, the method of the invention further comprises distillation to obtain the fermentation product, e.g., ethanol. The fermentation and the distillation may be carried out simultaneously and/or separately/sequentially; optionally followed by one or more process steps for further refinement of the fermentation product. Following the completion of the distillation process, the material remaining is considered the whole stillage.

As another example, the desired fermentation product may be extracted from the fermentation medium by micro or membrane filtration techniques. Ethanol with a purity of up to about 96 vol. % can be obtained, which can be used as, for example, fuel ethanol, drinking ethanol, i.e., potable neutral spirits, or industrial ethanol.

In some embodiments of the methods, the fermentation product after being recovered is substantially pure. With respect to the methods herein, “substantially pure” intends a recovered preparation that contains no more than 15% impurity, wherein impurity intends compounds other than the fermentation product (e.g., ethanol). In one variation, a substantially pure preparation is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

Suitable assays to test for the production of ethanol and contaminants, and sugar consumption can be performed using methods known in the art. For example, ethanol product, as well as other organic compounds, can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of ethanol in the fermentation broth can also be tested with the culture supernatant. Byproducts and residual sugar in the fermentation medium (e.g., glucose or xylose) can be quantified by HPLC using, for example, a refractive index detector for glucose and alcohols, and a UV detector for organic acids (Lin et al., Biotechnol. Bioeng. 90:775-779 (2005)), or using other suitable assay and detection methods well known in the art.

### B. Processing of Whole Stillage

In one embodiment, the whole stillage is processed into two streams—wet cake and centrate. The whole stillage is separated or partitioned into a solid and liquid phase by one or more methods for separating the centrate from the wet cake. The centrate is split into two flows—thin stillage, which goes to the evaporators, and backset, which is recycled to the front of the plant. Separating whole stillage into centrate (e.g., thin stillage when pumped toward the evaporators rather than the front end of the plant) and wet cake to remove a significant portion of the liquid/water, may be done using any suitable separation technique, including centrifugation, pressing and filtration. In a preferred embodiment, the separation/dewatering is carried out by centrifugation. Preferred centrifuges in industry are decanter type centrifuges, preferably high speed decanter type centrifuges. An example of a suitable centrifuge is the NX 400 steep cone series from ALFA LAVAL which is a high-performance decanter. A similar decanter centrifuge can also be purchased from FLOTTWEG. In another preferred embodiment, the separation is carried out using other conventional separation equipment such as a plate/frame filter presses, belt filter presses, screw presses, gravity thickeners and deckers, or similar equipment.

### C. Processing of Thin Stillage

Thin stillage is the term used for the supernatant of the centrifugation of the whole stillage. Typically, the thin stillage contains 4-8 percent dry solids (DS) (mainly proteins, soluble fiber, fats, fine fibers, and cell wall components) and has a temperature of about 60-90 degrees centigrade. The thin stillage stream may be condensed by evaporation to provide two process streams including: (i) an evaporator condensate stream comprising condensed water removed from the thin stillage during evaporation, and (ii) a syrup stream, comprising a more concentrated stream of the non-volatile dissolved and non-dissolved solids, such as non-fermentable sugars and oil, remaining present from the thin stillage as the result of removing the evaporated water.

Optionally, oil can be removed from the thin stillage or can be removed as an intermediate step to the evaporation process, which is typically carried out using a series of several evaporation stages.

Syrup and/or de-oiled syrup may be introduced into a dryer together with the wet cake (from the whole stillage separation step) to provide a product referred to as distillers dried grain with solubles, which also can be used as animal feed. In an embodiment, syrup and/or de-oiled syrup is sprayed into one or more dryers to combine the syrup and/or de-oiled syrup with the whole stillage to produce distillers dried grain with solubles.

Between 5-90 vol-%, such as between 10-80%, such as between 15-70%, such as between 20-60% of thin stillage (e.g., optionally hydrolyzed) may be recycled (as backset) to step (a). The recycled thin stillage (i.e., backset) may constitute from about 1-70 vol.-%, preferably 15-60% vol.-%, especially from about 30 to 50 vol.-% of the slurry formed in step (a). In an embodiment, the process further comprises recycling at least a portion of the thin stillage stream to the slurry, optionally after oil has been extracted from the thin stillage stream.

D. Drying of Wet Cake and Producing Distillers Dried Grains and Distillers Dried Grains with Solubles

After the wet cake, containing about 25-40 wt-%, preferably 30-38 wt-% dry solids, has been separated from the thin stillage (e.g., dewatered) it may be dried in a drum dryer, spray dryer, ring drier, fluid bed drier or the like in order to produce “Distillers Dried Grains” (DDG). DDG is a valuable feed ingredient for animals, such as livestock, poultry and fish. It is preferred to provide DDG with a content of less than about 10-12 wt.-% moisture to avoid mold and microbial breakdown and increase the shelf life. Further, high moisture content also makes it more expensive to transport DDG. The wet cake is preferably dried under conditions that do not denature proteins in the wet cake. The wet cake may be blended with syrup separated from the thin stillage and dried into DDG with Solubles (DDGS). Partially dried intermediate products, such as are sometimes referred to as modified wet distillers grains, may be produced by partially drying wet cake, optionally with the addition of syrup before, during or after the drying process.

### Fermentation Broth Formulations or Cell Compositions

The present invention also relates to a fermentation broth formulation or a cell composition comprising a polypeptide of the present invention. The fermentation broth formulation or the cell composition further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or cell composition may contain the unfractionated contents of the fermentation materials derived at the end of the fermentation. Typically, the cell-killed whole broth or cell composition contains the spent culture medium and cell debris present after the microbial cells (e.g., filamentous fungal cells) are grown to saturation, incubated under carbon-limiting conditions to allow protein synthesis. In some embodiments, the cell-killed whole broth or cell composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

The present invention is further described by the following examples that should not be construed as limiting the scope of the invention.

### EXAMPLES

### Enzymes Used in the Examples

CE1A: exemplary CE1 family polypeptide having ferulic acid esterase and/or acetyl xylan esterase activity from Microsphaeropsis arundinis disclosed in SEQ ID NO: 3.

CE1B: exemplary CE1 family polypeptide having ferulic acid esterase and/or acetyl xylan esterase activity from Microsphaeropsis arundinis disclosed in SEQ ID NO: 6.

CE1C: exemplary CE1 family polypeptide having ferulic acid esterase and/or acetyl xylan esterase activity from Microsphaeropsis arundinis disclosed in SEQ ID NO: 9.

CE1D: exemplary CE1 family polypeptide having ferulic acid esterase and/or acetyl xylan esterase activity from Microsphaeropsis arundinis disclosed in SEQ ID NO: 12.

CE1E: CE1 family polypeptide having ferulic acid esterase and/or acetyl xylan esterase activity from Microsphaeropsis arundinis disclosed in SEQ ID NO: 15.

GH43A: exemplary GH43 arabinofuranosidase from Humicola insolens disclosed in SEQ ID NO: 16.

GH43B: exemplary GH43 arabinofuranosidase from Lasiodiplodia theobromane disclosed in SEQ ID NO: 17.

GH43C: exemplary GH43 arabinofuranosidase from Poronia punctata disclosed in SEQ ID NO: 18.

GH51A: exemplary GH51 arabinofuranosidase from Meripilus giganteus disclosed in SEQ ID NO: 19.

GH51B: exemplary GH51 arabinofuranosidase from Lasiodiplodia theobromae disclosed in SEQ ID NO: 20.

GH51C: exemplary GH51 arabinofuranosidase from Acidiella bohemica disclosed in SEQ ID NO: 21.

GH5_21A: exemplary GH5_21 xylanase from Bacteroides cellulosilyticus CL02T12C19 disclosed in SEQ ID NO: 22.

GH5_21B: exemplary GH5_21 xylanase from Xanthan alkaline community S disclosed in SEQ ID NO: 23.

GH5_21C: exemplary GH5_21 xylanase from Sphingobacterium sp-64162 disclosed in SEQ ID NO: 24.

GH5_21D: exemplary GH5_21 xylanase from Sphingobacterium sp-64162 disclosed in SEQ ID NO: 25.

GH5_21E: exemplary GH5_21 xylanase from Xanthan alkaline community O disclosed in SEQ ID NO: 26.

GH5_21F: exemplary GH5_21 xylanase from bioreactor metagenome disclosed in SEQ ID NO: 27.

GH5_21G: exemplary GH5_21 xylanase from Xanthan alkaline community T disclosed in SEQ ID NO: 28.

GH5_21H: exemplary GH5_21 xylanase from Xanthan alkaline community S disclosed in SEQ ID NO: 29.

GH5_21I: exemplary GH5_21 xylanase from Belliella sp-64282 disclosed in SEQ ID NO: 30.

GH5_21J: exemplary GH5_21 xylanase from Chryseobacterium oncorhynchi disclosed in SEQ ID NO: 31.

GH5_21K: exemplary GH5_21 xylanase from Xanthan alkaline community T disclosed in SEQ ID NO: 32.

GH5_21L: exemplary GH5_21 xylanase from Sphingobacterium disclosed in SEQ ID NO: 33.

GH5_21M: exemplary GH5_21 xylanase from elephant dung metagenome disclosed in SEQ ID NO:34.

GH5_21 N: exemplary GH5_21 xylanase from elephant dung metagenome disclosed in SEQ ID NO: 35.

GH5_21O: exemplary GH5_21 xylanase from Chryseobacterium sp disclosed in SEQ ID NO: 36.

GH5_35A: exemplary GH5_35 xylanase from Cohnella xylanilytica disclosed in SEQ ID NO: 37.

GH5_35B: exemplary GH5_35 xylanase from Bacillus hemicellulosilyticus JCM 9152 disclosed in SEQ ID NO: 38.

GH5_35C: exemplary GH5_35 xylanase from Paenibacillus sp-62332 disclosed in SEQ ID NO: 39.

GH5_35D: exemplary GH5_35 xylanase from compost metagenome disclosed in SEQ ID NO: 40.

GH5_35E: exemplary GH5_35 xylanase from Paenibacillus chitinolyticus disclosed in SEQ ID NO: 41.

GH30_8: exemplary GH30_8 xylanase from Bacillus sp-18423 disclosed in SEQ ID NO: 42.

GH3A: exemplary GH3 beta-xylosidase from Aspergillus fumigatus disclosed in SEQ ID NO: 43.

GH3B: exemplary GH3 beta-xylosidase from Aspergillus nidulans disclosed in SEQ ID NO: 44.

GH3C: exemplary GH3 beta-xylosidase from Talaromyces emersonii disclosed in SEQ ID NO: 45.

GH31A: exemplary GH31 alpha-xylosidase from Herbinix hemicellulosilytica disclosed in SEQ ID NO: 46

GH8: exemplary GH8 xylanase from Bacillus sp. KK-1 disclosed in SEQ ID NO: 47.

GH10: exemplary GH10 xylanase from Aspergillus aculeatus disclosed in SEQ ID NO: 48.

GH11: exemplary GH11 xylanase from Thermomyces lanuginosus disclosed in SEQ ID NO: 49.

Liquefaction Enzyme Blend 1: exemplary thermostable alpha-amylase from Bacillus stearothermophilus disclosed in SEQ ID NO: 50; exemplary thermostable protease from Pyrococcus furiosus disclosed in SEQ ID NO: 52.

Liquefaction Enzyme Blend 2: exemplary thermostable alpha-amylase from Bacillus stearothermophilus disclosed in SEQ ID NO: 51; exemplary thermostable protease from Pyrococcus furiosus disclosed in SEQ ID NO: 52; exemplary thermostable xylanase from Thermotoga maritima disclosed in SEQ ID NO: 53.

Saccharification Enzyme Blend: exemplary glucoamylase from Gloeophyllum sepiarium disclosed in SEQ ID NO: 54; exemplary alpha-amylase from Rhizomucor pusillus disclosed in SEQ ID NO: 55; exemplary trehalase from Talaromyces funiculosus disclosed in SEQ ID NO: 56; exemplary beta-glucosidase from Aspergillus fumigatus disclosed in SEQ ID NO: 57; exemplary celliobiohydrolase from Aspergillus fumigatus disclosed in SEQ ID NO: 58; exemplary endoglucanase from Trichoderma reesei disclosed in SEQ ID NO: 59.

Hemicellulase Blend: polypeptide having arabinofuranosidase activity on disubstituted arabinose (GH43A), polypeptide having arabinofuranosidase activity on monosubstituted arabinose (GH51A), polypeptide having xylanase activity (GH5_21O), and a polypeptide having beta-xylosidase activity (GH3A).

### Determination of Td by Differential Scanning Calorimetry for Liquefaction Enzymes

The thermostability of an enzyme is determined by Differential Scanning Calorimetry (DSC) using a VP-Capillary Differential Scanning Calorimeter (MicroCal Inc., Piscataway, NJ, USA). The thermal denaturation temperature, Td (° C.), is taken as the top of denaturation peak (major endothermic peak) in thermograms (Cp vs. T) obtained after heating enzyme solutions (approx. 0.5 mg/ml) in buffer (50 mM acetate, pH 5.0) at a constant programmed heating rate of 200 K/hr.

Sample- and reference-solutions (approx. 0.2 ml) are loaded into the calorimeter (reference: buffer without enzyme) from storage conditions at 10° C. and thermally pre-equilibrated for 20 minutes at 20° C. prior to DSC scan from 20° C. to 120° C. Denaturation temperatures are determined at an accuracy of approximately +/−1° C.

### Strains

The fungal strain NN074640 was isolated from the soil sample collected from Brazil in 2019 by the dilution plate method with PDA medium, pH6, 28° C. It was then purified by transferring a single conidium onto a PDA agar plate. The strain NN074640 was identified as Microsphaeropsis arundinis, based on both morphological characteristics and ITS rDNA sequence.

Yeast strain MEJI797 is MBG5012 of WO2019/161227 further expressing a Pycnopous sanguineus glucoamylase (SEQ ID NO: 4 of WO2011/066576) and a hybrid Rhizomucor pusillus alpha amylase expression cassette (as described in WO2013/006756).

### Media and Solutions

PDA plates were composed of 39 grams of potato dextrose agar and deionized water to 1 liter.

### EXAMPLES

### Example 1: Effect of Xylanases from GH Families 5, 8, 10, 11 and 30 in Combination with Arabinofuranosidases from GH Families 43 and 51 for Increasing Xylose and Arabinose in Simultaneous Saccharification and Fermentation Process

An industrial liquefied mash prepared using Liquefaction Enzyme Blend 1 was used for the experiment. The dry solid determined by moisture balance was about 34% DS and pH was adjusted to pH 5.0 following by supplemented with 3 ppm of penicillin and 500 ppm of urea. Simultaneous saccharification and fermentation (SSF) was performed via mini-scale fermentations. Approximately 4.0 g of the industrial liquefied corn mash was added to 15 ml tube vials. Each vial was dosed with 0.42 AGU/gDS of Saccharification Enzyme Blend and appropriate amount of respective xylanase and arabinofuranosidase with the dosing scheme treatments as shown in Table 2. Saccharification Enzyme blend was used as a control, without addition of xylanase or arabinofuranosidase. Actual enzymes dosages were based on the exact weight of corn slurry in each vial. After enzymes addition, fermentation was initiated by addition of 50 μL of propagated yeast strain MEJI797. Tubes were incubated at 32° C. with three replicates for each treatment. After 65 hours SSF, tubes were taken out from incubator and added 50 μL of 34% H2SO4 and then subjected to centrifugation 3500 rpm for 10 min follow by filtering through a 0.45 micrometer filter. Sugars concentrations were determined using HPLC system equipped with H column (Benson Polymeric, BP-700 H, 300×7.8 mm).

TABLE 2
Dosing scheme of arabinofuranosidase with or without xylanase

| | GH43 | Dose, | GH51 | Dose, | | Dose, |
|---|---|---|---|---|---|---|
| Treatment | Araf | ug/gDS | Araf | ug/gDS | Xylanase | ug/gDS |
| Control | | | | | | |
| GH43A | GH43 | 10 | | | | |
| GH51A | | | GH51 | 10 | | |
| GH43A + GH51A | GH43 | 10 | GH51 | 10 | | |
| GH43A + GH51A + GH5_21O | GH43 | 10 | GH51 | 10 | GH5_21 | 10 |
| GH43A + GH51A + GH8 | GH43 | 10 | GH51 | 10 | GH8 | 10 |
| GH43A + GH51A + GH10 | GH43 | 10 | GH51 | 10 | GH10 | 10 |
| GH43A + GH51A + GH11 | GH43 | 10 | GH51 | 10 | GH11 | 10 |
| GH43A + GH51A + GH30_8 | GH43 | 10 | GH51 | 10 | GH30_8 | 10 |

TABLE 3
Results

| | Mean arabinose, | % Boost |
|---|---|---|
| Treatment | ppm | arabinose |
| Control | 490 | 0% |
| GH43A | 770 | 57% |
| GH51A | 770 | 57% |
| GH43A + GH51A | 1190 | 142% |
| GH43A + GH51A + GH5_21O | 2270 | 363% |
| GH43A + GH51A + GH8 | 1350 | 175% |
| GH43A + GH51A + GH10 | 1330 | 172% |
| GH43A + GH51A + GH11 | 1270 | 159% |
| GH43A + GH51A + GH30_8 | 2150 | 338% |

% Boost arabinose = [(Mean arabinose experimental ppm − mean arabinose control)/mean arabinose control] × 100

Table 3 shows that GH5_21 or GH30_8 xylanases combined with GH43 and GH51 arabinofuranosidases release the highest concentration of arabinose compared to GH43 or GH51 arabinofuranosidase alone or their combination without xylanase.

### Example 2: Effect of GH3 Family Beta-Xylosidase Combination with Arabinofuranosidase from GH 43 and 51 Families and Xylanase from GH5_21 for Increasing Xylose in Simultaneous Saccharification and Fermentation Process

An industrial liquefied mash prepared using Liquefaction Enzyme Blend 2 was used for the experiment. The dry solid determined by moisture balance was about 35.9% DS and pH was adjusted to pH 5.0 following by supplemented with 3 ppm of penicillin and 500 ppm of urea. Simultaneous saccharification and fermentation (SSF) was performed via mini-scale fermentations. Approximately 4.2 g of the industrial liquefied corn mash was added to 15 ml tube vials. Each vial was dosed with 0.42 AGU/gDS of Saccharification Enzyme Blend and appropriate amount of respective xylanase, arabinofuranosidase and beta-xylosidase as listed in Table 4. The dosing scheme followed the fixed amount of GH5_21 xylanase, GH43 arabinofuranosidase and GH51 arabinofuranosidase of each 10 ug/g dry solids, respectively, with or without beta-xylosidase GH3A, GH3B or GH3C at a dosage of 25, 50, 100 or 200 ug/g dry solids. Saccharification Enzyme Blend was used as a control, without addition of xylanases or beta-xylosidases. Actual enzymes dosages were based on the exact weight of corn slurry in each vial. After enzymes addition, fermentation was initiated by addition of 50 μL of propagated yeast strain MEJI797. Tubes were incubated at 32° C. with three replicates for each treatment. After 65 hours SSF, tubes were taken out from incubator and then subjected to centrifugation 3500 rpm for 10 min follow by filtering through a 0.45 micrometer filter. Sugars concentrations were determined using HPLC system equipped with lead column (Benson Polymeric, BP-800 Pb, 300×7.8 mm).

### Result

Table 4 shows beta-xylosidase combined with GH43, GH51 arabinofuranosidases and GH5_21 xylanase significantly increases xylose release and higher enzyme dosages corresponded to higher xylose release.

TABLE 4

| | | | % Boost xylose, |
|---|---|---|---|
| | Mean | % Boost | ref GH43 + |
| | xylose, | xylose, | GH51 + GH5-21 |
| Treatment (ug/gDS) | ppm | ref control | (without GH3) |
| Control | 390 | 0% | |
| GH43A + GH51O + GH5-21O | 870 | 121% | 0% |
| GH43A + GH51O + GH5-21O + GH3A (25) | 1300 | 230% | 49% |
| GH43A + GH51O + GH5-21O + GH3A (50) | 1390 | 251% | 60% |
| GH43A + GH51O + GH5-21O + GH3A (100) | 1510 | 283% | 74% |
| GH43A + GH51O + GH5-21O + GH3A (200) | 1640 | 315% | 89% |
| GH43A + GH51O + GH5-21O + GH3B (25) | 1120 | 183% | 29% |
| GH43A + GH51O + GH5-21O + GH3B (50) | 1170 | 196% | 34% |
| GH43A + GH51O + GH5-21O + GH3B (100) | 1280 | 223% | 47% |
| GH43A + GH51O + GH5-21O + GH3B (200) | 1420 | 260% | 63% |
| GH43A + GH51O + GH5-21O + GH3C (25) | 1040 | 163% | 20% |
| GH43A + GH51O + GH5-21O + GH3C (50) | 1060 | 169% | 22% |
| GH43A + GH51O + GH5-21O + GH3C (100) | 1120 | 184% | 29% |
| GH43A + GH51O + GH5-21O + GH3C (200) | 1190 | 202% | 37% |

### Example 3: Effect of GH 5 Xylanase Subfamilies 21 and 35 Combination with Hi GH 43 and Mg GH51 Arabinofuranosidases and Af GH3 Beta-Xylosidase for Increasing Xylose and Arabinose in Simultaneous Saccharification and Fermentation Process

An industrial liquefied mash prepared using Liquefaction Enzyme Blend 1 was used for the experiment. The dry solid determined by moisture balance was about 33.7% DS and pH was adjusted to pH 5.0 following by supplemented with 3 ppm of penicillin and 500 ppm of urea. Simultaneous saccharification and fermentation (SSF) was performed via mini-scale fermentations. Approximately 4.2 g of the industrial liquefied corn mash was added to 15 ml tube vials. Each vial was dosed with 0.42 AGU/gDS of Saccharification Enzyme Blend, 10 ug/gDS of GH43A arabinofuranosidase, 10 ug/gDS of GH51A arabinofuranosidase, 25 ug/gDS of GH3A beta-xylosidase and 10 ug/gDS of respective xylanase as listed in Table 5. Saccharification Enzyme Blend was used as a control, without addition of arabinofuranosidases, xylanases or beta-xylosidases. Actual enzymes dosages were based on the exact weight of corn slurry in each vial. After enzymes addition, fermentation was initiated by addition of 50 μL of propagated yeast strain MEJI797. Tubes were incubated at 32° with three replicates for each treatment. After 65 hours SSF, tubes were taken out from incubator and added 50 μL of 34% H2SO4 and then subjected to centrifugation 3500 rpm for 10 min follow by filtering through a 0.45 micrometer filter. Sugars concentrations were determined using HPLC system equipped with lead column (Benson Polymeric, BP-800 Pb, 300×7.8 mm).

### Result

Table 5 shows that the addition of xylanases from GH5_21 and GH5035 significantly increase xylose and arabinose release compared to control or treatment consist of GH43, GH51 arabinofuranosidase and GH3 beta-xylosidase, without xylanase.

TABLE 5

| | Mean | Mean |
|---|---|---|
| | xylose, | arabinose, |
| Treatment | ppm | ppm |
| Control | 340 | 150 |
| GH43A + GH51A + GH3A | 720 | 920 |
| GH43A + GH51A + GH3A + GH5_21A | 830 | 1150 |
| GH43A + GH51A + GH3A + GH5_21B | 820 | 1300 |
| GH43A + GH51A + GH3A + GH5_21C | 990 | 1740 |
| GH43A + GH51A + GH3A + GH5_21D | 980 | 1680 |
| GH43A + GH51A + GH3A + GH5_21E | 880 | 1240 |
| GH43A + GH51A + GH3A + GH5_21F | 910 | 1360 |
| GH43A + GH51A + GH3A + GH5_21G | 920 | 1390 |
| GH43A + GH51A + GH3A + GH5_21H | 870 | 1200 |
| GH43A + GH51A + GH3A + GH5_21I | 1050 | 1370 |
| GH43A + GH51A + GH3A + GH5_21J | 1130 | 1690 |
| GH43A + GH51A + GH3A + GH5_21K | 990 | 1260 |
| GH43A + GH51A + GH3A + GH5_21L | 1150 | 1740 |
| GH43A + GH51A + GH3A + GH5_21M | 1200 | 1770 |
| GH43A + GH51A + GH3A + GH5_21N | 1240 | 1850 |
| GH43A + GH51A + GH3A + GH5_21O | 1090 | 1890 |
| GH43A + GH51A + GH3A + GH5_35A | 1120 | 1600 |
| GH43A + GH51A + GH3A + GH5_35B | 1140 | 1580 |
| GH43A + GH51A + GH3A + GH5_35C | 1110 | 1560 |
| GH43A + GH51A + GH3A + GH5_35D | 1150 | 1630 |
| GH43A + GH51A + GH3A + GH5_35E | 1120 | 1580 |

### Example 4: Effect of Single, Double, Triple or Quadruple Combination of Hemicellulases of GH5_21 Xylanase, GH43 and GH51 Arabinofuranosidase and GH3 Beta-Xylosidase for Increasing Xylose and Arabinose in Simultaneous Saccharification and Fermentation Process

An industrial liquefied mash prepared using Liquefaction Enzyme Blend 2 was used for the experiment. The dry solid determined by moisture balance was about 33.4% DS and pH was adjusted to pH 5.0 following by supplemented with 3 ppm of penicillin and 500 ppm of urea. Simultaneous saccharification and fermentation (SSF) was performed via mini-scale fermentations. Approximately 4.2 g of the industrial liquefied corn mash was added to 15 ml tube vials. Each vial was dosed with 0.6 AGU/gDS of Saccharification Enzyme Blend and followed the dosing scheme of 10 ug/gDS GH5_21O xylanase, 10 ug/gDS of GH43A arabinofuranosidase, 10 ug/gDS of GH51A arabinofuranosidase and/or 25 ug/gDS of GH3A beta-xylosidase. As control, only Saccharification Enzyme Blend was added without arabinofuranosidases, xylanases or beta-xylosidases. Actual enzymes dosages were based on the exact weight of corn slurry in each vial. After enzymes addition, fermentation was initiated by addition of 50 μL of hydrated yeast strain MEJI797. Tubes were incubated at 32° C. with three replicates for each treatment. After 65 hours SSF, tubes were taken out from incubator and added 50 μL of 34% H2SO4 and then subjected to centrifugation 3500 rpm for 10 min follow by filtering through a 0.45 micrometer filter. Sugars concentrations were determined using HPLC system equipped with H column (Benson Polymeric, BP-700 H, 300×7.8 mm). The decanted tubes containing wet corn mash at the end of fermentation were taken to vacuum freeze drying for 3 days. The dried solids weight of each tube was determined, and residual solids were calculated as ratio of final solid weight over the initial solid weight.

### Result

TABLE 6

| | Mean | Mean | Mean residual |
|---|---|---|---|
| | xylose, | arabinose, | solids, |
| Treatment | ppm | ppm | % |
| Control | 277 | 294 | 27% |
| GH5_21O | 296 | 317 | 25% |
| GH43A | 272 | 665 | 26% |
| GH51A | 268 | 822 | 25% |
| GH3A | 293 | 294 | 25% |
| GH43A + GH51A | 264 | 1362 | 25% |
| GH43A + GH3A | 260 | 679 | 25% |
| GH51 + GH3 | 235 | 826 | 25% |
| GH5_21O + GH43A + GH51A | 275 | 1967 | 23% |
| GH5_21O + GH43A + GH3A | 305 | 871 | 24% |
| GH5_21O + GH51A + GH3A | 609 | 1245 | 24% |
| GH43A + GH51A + GH3A | 616 | 1369 | 24% |
| GH5_21O + GH43A + GH51A + | 818 | 2030 | 24% |
| GH3A | | | |

Table 6 shows that the addition of GH5_21 xylanase together with GH43, GH51 arabinofuranosidase and GH3 beta-xylosidase increase xylose and arabinose release compared to control or treatment consist of GH43, GH51 arabinofuranosidase and GH3 beta-xylosidase, without xylanase.

### Example 5: Effect of Arabinofuranosidase from GH Families 43, and 51 Combinations with Xylanase from GH Family 5 Subfamily 21 for Increasing Arabinose in Simultaneous Saccharification and Fermentation Process

An industrial liquefied mash prepared using Liquefaction Enzyme Blend 2 was used for the experiment. The dry solid determined by moisture balance was about 36% DS and pH was adjusted to pH 5.0 following by supplemented with 3 ppm of penicillin and 500 ppm of urea. Simultaneous saccharification and fermentation (SSF) was performed via mini-scale fermentations. Approximately 4.0 g of the industrial liquefied corn mash was added to 15 ml tube vials. Each vial was dosed with 0.42 AGU/gDS of Saccharification Enzyme Blend and appropriate amount of arabinofuranosidase combinations from families GH43 and GH51, as listed in Table 7. The dosing scheme followed the fixed amount of GH5_21O xylanase, GH43 arabinofuranosidase and GH51 arabinofuranosidase of each 10 ug/g dry solids, respectively. As control, only Saccharification Enzyme Blend was used with no addition of xylanase or arabinofuranosidase. Actual enzymes dosages were based on the exact weight of corn slurry in each vial. After enzymes addition, fermentation was initiated by addition of 50 μL of propagated yeast strain MEJI797. Tubes were incubated at 32° C. with three replicates for each treatment. After 65 hours SSF, tubes were taken out from incubator and added 50 μL of 34% H2SO4 and then subjected to centrifugation 3500 rpm for 10 min follow by filtering through a 0.45 micrometer filter. Sugars concentrations were determined using HPLC system equipped with H column (Benson Polymeric, BP-700 H, 300×7.8 mm).

### Result

TABLE 7

| Treatment | Mean arabinose, ppm | % Boost arabinose |
|---|---|---|
| Control | 165 | 0 |
| GH43A + GH51A | 2744 | 1563% |
| GH43B + GH51A | 2559 | 1451% |
| GH43A + GH51C | 1842 | 1016% |
| GH43A + GH51B | 2198 | 1232% |

% Boost arabinose = [(Mean arabinose experimental ppm − mean arabinose control)/mean arabinose control] × 100

Table 7 shows that GH43 and GH51 arabinofuranosidases in combination with GH5_21 xylanases increase arabinose compared to control without arabinofuranosidases and xylanase.

### Example 6: Effect of Arabinofuranosidase from GH Families 43, and 51 Combination with Xylanase from GH Family 5 Subfamily 21 for Increasing Arabinose in Simultaneous Saccharification and Fermentation Process

An industrial liquefied mash prepared using Liquefaction Enzyme Blend 2 was used for the experiment. The dry solid determined by moisture balance was about 33.8% DS and pH was adjusted to pH 5.0 following by supplemented with 3 ppm of penicillin and 500 ppm of urea. Simultaneous saccharification and fermentation (SSF) was performed via mini-scale fermentations. Approximately 4.2 g of the industrial liquefied corn mash was added to 15 ml tube vials. Each vial was dosed with 0.42 AGU/gDS of Saccharification Enzyme Blend and appropriate amount of arabinofuranosidase combination from families GH43 and GH51, as listed in Table 8. The dosing scheme followed the fixed amount of GH5_21 xylanase, GH43 arabinofuranosidase and GH51 arabinofuranosidase of each 10 ug/g dry solids, respectively. As control, only Saccharification Enzyme Blend with no addition of xylanase or arabinofuranosidase. Actual enzymes dosages were based on the exact weight of corn slurry in each vial. After enzymes addition, fermentation was initiated by addition of 50 μL of propagated yeast strain MEJI797. Tubes were incubated at 32° C. with three replicates for each treatment. After 65 hours SSF, tubes were taken out from incubator and added 50 μL of 34% H₂SO4 and then subjected to centrifugation 3500 rpm for 10 min follow by filtering through a 0.45 micrometer filter. Sugars concentrations were determined using HPLC system equipped with H column (Benson Polymeric, BP-700 H, 300×7.8 mm).

### Result

TABLE 8

| Treatment | Mean arabinose, ppm | % Boost arabinose |
|---|---|---|
| Control | 415 | 0% |
| GH43A + GH51A | 2120 | 410% |
| GH43C + GH51A | 1902 | 358% |

% Boost arabinose = [(Mean arabinose experimental ppm − mean arabinose control)/mean arabinose control] × 100

Table 8 shows that GH43 and GH1 arabinofuranosidases in combination with GH5_21 xylanases increase arabinose release compared to without xylanase and arabinofuranosidases.

### Example 7: Effect of Exemplary CE1 Polypeptides Having Ferulic Acid Esterase and/or Acetyl Xylan Esterase Activity from Microsphaeropsis arundinis in Combination with Exemplary Polypeptide Having Arabinofuranosidase Activity on Disubstituted Arabinose, Polypeptide Having Arabinofuranosidase Activity on Monosubstituted Arabionose, a Polypeptide Having Xylanase Activity and a Polypeptide Having Beta-Xylosidase Activity for Increasing Xylose and Arabinose in Simultaneous Saccharification and Fermentation Process

An industrial liquefied mash prepared using Liquefaction Enzyme Blend 1 was used for the experiment. The dry solid determined by moisture balance was about 34% DS and pH was adjusted to pH 5.0 following by supplemented with 3 ppm of penicillin and 500 ppm of urea. Simultaneous saccharification and fermentation (SSF) was performed via mini-scale fermentations. Approximately 4.2 g of the industrial liquefied corn mash was added to 10 ml tube vials. Each vial was dosed with 0.42 AGU/gDS of Saccharification Enzyme Blend together with amounts of a Hemicellulase Blend shown in Table 9 and 10 ug/gDS of respective CE1 polypeptides shown in Table 9 followed by addition of 50 μL of propagated yeast strain MEJI797 per 4.2 g slurry. As control, only Saccharification Enzyme Blend was used without addition of Hemicellulase Blend or CE1 polypeptide. Actual enzymes dosages were based on the exact weight of corn slurry in each vial. Vials were incubated at 32° C. with three replicates for each treatment. After 65 hours SSF, tubes were taken out from incubator and subjected to centrifugation 3500 rpm for 10 min follow by filtering through a 0.45 micrometer filter. Sugars concentrations were determined using HPLC system equipped with H column (Benson Polymeric, BP-700 H, 300×7.8 mm). The decanted tubes containing wet corn mash at the end of fermentation were taken to vacuum freeze drying for 3 days. The dried solids weight of each tube was determined, and residual solids were calculated as ratio of final solid weight over the initial solid weight.

TABLE 9
Dose of enzymes in hemicellulase blend

| Enzyme | Dose, ug/g dry solids |
|---|---|
| GH21O | 10 |
| GH43A | 10 |
| GH51A | 10 |
| GH3A | 25 |

### Result

As shown in Table 10 below, the combination of a CE1 polypeptide with a Hemicellulase Blend increases xylose and arabinose release compared to control or Hemicellulase Blend without addition of the CE1 polypeptide. The Hemicellulase Blend significantly reduced residual solids compared to control and the addition of the CE1 polypeptide further reduced the residual solids, indicating increased corn fiber degradation by the Hemicellulase Blend and its combination with the CE1 polypeptide.

TABLE 10

| | Xylose, | Arabinose, | Residual |
|---|---|---|---|
| Treatment | ppm | ppm | solids, % |
| Control | 230 | 240 | 24.1 |
| Hemicellulase Blend | 480 | 1980 | 22.9 |
| Hemicellulase Blend + CE1A | 490 | 1900 | 22.1 |
| Hemicellulase Blend + CE1B | 480 | 2010 | 22.1 |
| Hemicellulase Blend + CE1C | 480 | 2000 | 22.2 |
| Hemicellulase Blend + CE1D | 600 | 2300 | 22.6 |
| Hemicellulase Blend + CE1E | 520 | 2150 | 22.7 |

### Example 8: Effect of Exemplary CE1 Polypeptides Having Ferulic Acid Esterase and/or Acetyl Xylan Esterase Activity in Combination with Exemplary Polypeptide Having Arabinofuranosidase Activity on Disubstituted Arabinose, Exemplary Polypeptide Having Arabinofuranosidase Activity on Monosubstituted Arabionose, Exemplary Polypeptide Having Xylanase Activity and an Exemplary Polypeptide Having Beta-Xylosidase Activity with or without an Exemplary Alpha-Xylosidase for Increasing Xylose and Arabinose in Simultaneous Saccharification and Fermentation Process

An industrial liquefied mash prepared using Liquefaction Enzyme Blend 1 was used for the experiment. The dry solid determined by moisture balance was about 35% DS and pH was adjusted to pH 5.0 following by supplemented with 3 ppm of penicillin and 500 ppm of urea. Simultaneous saccharification and fermentation (SSF) was performed via mini-scale fermentations. Approximately 4.2 g of the industrial liquefied corn mash was added to 10 ml tube vials. Each vial was dosed with 0.42 AGU/gDS of Saccharification Enzyme Blend together with amounts of a Hemicellulase Blend shown in Table 9 above, and 10 ug/gDS of CE1D and CE1E polypeptides with or without 10 ug/gDS of GH31A, followed by addition of 50 μL of propagated yeast strain MEJI797 per 4.2 g slurry. As control, only Saccharification Enzyme Blend was used without the addition of Hemicellulase Blend, CE1 enzyme or GH31A. Actual enzymes dosages were based on the exact weight of corn slurry in each vial. Vials were incubated at 32° C. with three replicates for each treatment. After 65 hours SSF, tubes were taken out from incubator and subjected to centrifugation 3500 rpm for 10 min follow by filtering through a 0.45 micrometer filter. Sugars concentrations were determined using HPLC systems equipped with lead column (Benson Polymeric, BP-800 Pb, 300×7.8 mm). The decanted tubes containing wet corn mash at the end of fermentation were taken to vacuum freeze drying for 3 days. The dried solids weight of each tube was determined, and residual solids were calculated as ratio of final solid weight over the initial solid weight.

### Result

As shown in Table 11 below, the combination of the CE1 polypeptide with the Hemicellulase Blend increases release of xylose and arabinose compared to control the Hemicellulase Blend without the CE1 polypeptide. Addition of an exemplary alpha-xylosidase (e.g., GH31A) further increases xylose and arabinose release when mixed with the CE1 polypeptide and the Hemicellulase Blend. The Hemicellulase Blend significantly reduced residual solids compared to control and addition of CE1 polypeptide and alpha-xylosidase (e.g., GH31) further decreased residual solids, indicating increased corn fiber degradation by the Hemicellulase Blend and its combination with CE1 polypeptide and GH31 alpha-xylosidase.

TABLE 11

| | Xylose, | Arabinose, | Residual |
|---|---|---|---|
| Treatment | ppm | ppm | solids, % |
| Control | 490 | 280 | 22.9 |
| Hemicellulase Blend | 970 | 1640 | 21.4 |
| Hemicellulase Blend + GH31A | 960 | 1600 | 21.4 |
| Hemicellulase Blend + CE1D | 1040 | 1640 | 20.1 |
| Hemicellulase Blend + CE1E | 1000 | 1600 | 20.1 |
| Hemicellulase Blend + CE1D + GH31A | 1080 | 1630 | 19.9 |
| Hemicellulase Blend + CE1E + GH31A | 1050 | 1640 | 20.0 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered paragraphs:

1. A polypeptide having ferulic acid esterase and/or acetyl xylan esterase activity, selected from the group consisting of:
(i)
(a) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 2;
(b) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 3;
(c) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 2;
(d) a polypeptide encoded by a polynucleotide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 1;
(e) a polypeptide derived from SEQ ID NO: 2, a mature polypeptide of SEQ ID NO: 2, or SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity;
(ii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 5;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 6;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 5;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 4;
(e) a polypeptide derived from SEQ ID NO: 5, a mature polypeptide of SEQ ID NO: 5, or SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 8;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 9;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 8;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 7;
(e) a polypeptide derived from SEQ ID NO: 8, a mature polypeptide of SEQ ID NO: 8, or SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iv)
(a) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 11;
(b) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 12;
(c) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 11;
(d) a polypeptide encoded by a polynucleotide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 10;
(e) a polypeptide derived from SEQ ID NO: 11, a mature polypeptide of SEQ ID NO: 11, or SEQ ID NO: 12 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity; and
(v)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 14;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 15;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 14;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 13;
(e) a polypeptide derived from SEQ ID NO: 14, a mature polypeptide of SEQ ID NO: 14, or SEQ ID NO: 15 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity.

2. A polypeptide having ferulic acid esterase and/or acetyl xylan esterase activity, selected from the group consisting of:
(i)
(a) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 2;
(b) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 3;
(c) a polypeptide encoded by a polynucleotide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 1;
(d) a polypeptide derived from the polypeptide of (a), (b), (c), wherein the N- and/or C-terminal end has been extended by addition of one or more amino acids, said polypeptide having ferulic acid esterase and/or acetyl xylan esterase activity; and
(e) a fragment of the polypeptide of (a), (b), (c);
wherein the polypeptide has ferulic acid esterase and/or acetyl xylan esterase activity;
(ii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 5;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 6;
(c) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 4;
(d) a polypeptide derived from the polypeptide of (a), (b), or (c), wherein the N- and/or C-terminal end has been extended by addition of one or more amino acids, said polypeptide having ferulic acid esterase and/or acetyl xylan esterase activity; and
(e) a fragment of the polypeptide of (a), (b), (c);
wherein the polypeptide has ferulic acid esterase activity and/or acetyl xylan esterase activity;
(iii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 8;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 9;
(c) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 7;
(d) a polypeptide derived from the polypeptide of (a), (b), or (c), wherein the N- and/or C-terminal end has been extended by addition of one or more amino acids, said polypeptide having ferulic acid esterase and/or acetyl xylan esterase activity; and
(e) a fragment of the polypeptide of (a), (b), (c);
wherein the polypeptide has ferulic acid esterase activity and/or acetyl xylan esterase activity;
(iv)
(a) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 11;
(b) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 12;
(c) a polypeptide encoded by a polynucleotide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 10;
(d) a polypeptide derived from the polypeptide of (a), (b), or (c), wherein the N- and/or C-terminal end has been extended by addition of one or more amino acids, said polypeptide having ferulic acid esterase and/or acetyl xylan esterase activity; and
(e) a fragment of the polypeptide of (a), (b), (c);
wherein the polypeptide has ferulic acid esterase activity and/or acetyl xylan esterase activity; and
(v)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 14;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 15;
(c) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 13;
(d) a polypeptide derived from the polypeptide of (a), (b), or (c), wherein the N- and/or C-terminal end has been extended by addition of one or more amino acids, said polypeptide having ferulic acid esterase and/or acetyl xylan esterase activity; and
(e) a fragment of the polypeptide of (a), (b), (c);
wherein the polypeptide has ferulic acid esterase activity and/or acetyl xylan esterase activity.

3. A polypeptide having ferulic acid esterase and/or acetyl xylan esterase activity, which is selected from the group consisting of:
(i)
(a) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 2; or
(b) a fragment of the polypeptide of (a);
   wherein the polypeptide has ferulic acid esterase and/or acetyl xylan esterase activity;
(ii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 5; or
(b) a fragment of the polypeptide of (a);
   wherein the polypeptide has ferulic acid esterase and/or acetyl xylan esterase activity;
(iii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 8; or
   (b) a fragment of the polypeptide of (a);
   wherein the polypeptide has ferulic acid esterase and/or acetyl xylan esterase activity;
(iv)
(a) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 11; or
   (b) a fragment of the polypeptide of (a);
   wherein the polypeptide has ferulic acid esterase and/or acetyl xylan esterase activity; and
(v)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 14; or
   (b) a fragment of the polypeptide of (a);
wherein the polypeptide has ferulic acid esterase and/or acetyl xylan esterase activity.

4. A polypeptide having ferulic acid esterase and/or acetyl xylan esterase activity, which is selected from the group consisting of:
(i)
(a) a polypeptide having at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 3; or
(b) a fragment of the polypeptide of (a);
wherein the polypeptide has ferulic acid esterase and/or acetyl xylan esterase activity;
(ii)
(a) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 6; or
(b) a fragment of the polypeptide of (a);
wherein the polypeptide has ferulic acid esterase and/or acetyl xylan esterase activity;
(iii)
(a) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 9; or
(b) a fragment of the polypeptide of (a);
wherein the polypeptide has ferulic acid esterase and/or acetyl xylan esterase activity;
(iv)
(a) a polypeptide having at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 12; or
(b) a fragment of the polypeptide of (a);
wherein the polypeptide has ferulic acid esterase and/or acetyl xylan esterase activity; and
(v)
(a) a polypeptide having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 15; or
(b) a fragment of the polypeptide of (a);
wherein the polypeptide has ferulic acid esterase and/or acetyl xylan esterase activity.

5. A polypeptide having ferulic acid esterase and/or acetyl xylan esterase activity, which is selected from the group consisting of:
(i)
(a) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 2; or
(b) a fragment of the polypeptide of (a);
   wherein the polypeptide has ferulic acid esterase and/or acetyl xylan esterase activity;
(ii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 5; or
(b) a fragment of the polypeptide of (a);
   wherein the polypeptide has ferulic acid esterase and/or acetyl xylan esterase activity;
(iii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 8; or
   (b) a fragment of the polypeptide of (a);
   wherein the polypeptide has ferulic acid esterase and/or acetyl xylan esterase activity;
(iv)
(a) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 11; or
   (b) a fragment of the polypeptide of (a);
   wherein the polypeptide has ferulic acid esterase and/or acetyl xylan esterase activity; and
(v)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 14; or
   (b) a fragment of the polypeptide of (a);
wherein the polypeptide has ferulic acid esterase and/or acetyl xylan esterase activity.

6. A polypeptide having ferulic acid esterase and/or acetyl xylan esterase activity, which is:
(a) a fragment of SEQ ID NO: 2 or 3, wherein the fragment preferably contains at least 235 amino acid residues (e.g., amino acids 20 to 255 of SEQ ID NO: 2 or amino acids 1 to 235 of SEQ ID NO: 3), at least 248 amino acid residues (e.g., amino acids 20 to 268 of SEQ ID NO: 2 or amino acids 1 to 248 of SEQ ID NO: 3), or at least 262 amino acid residues (e.g., amino acids 20 to 282 of SEQ ID NO: 2 or amino acids 1 to 262 of SEQ ID NO: 3), and wherein the fragment has ferulic acid esterase and/or acetyl xylan esterase activity;
(b) a fragment of SEQ ID NO: 5 or 6, wherein the fragment preferably contains at least 219 amino acid residues (e.g., amino acids 25 to 244 of SEQ ID NO: 5 or amino acids 1 to 219 of SEQ ID NO: 6), at least 232 amino acid residues (e.g., amino acids 25 to 257 of SEQ ID NO: 5 or amino acids 1 to 232 of SEQ ID NO: 6), or at least 245 amino acid residues (e.g., amino acids 25 to 270 of SEQ ID NO: 5 or amino acids 1 to 245 of SEQ ID NO: 6), and wherein the fragment has ferulic acid esterase and/or acetyl xylan esterase activity;
(c) a fragment of SEQ ID NO: 8 or 9, wherein the fragment preferably contains at least 247 amino acid residues (e.g., amino acids 21 to 268 of SEQ ID NO: 8 or amino acids 1 to 247 of SEQ ID NO: 9), at least 261 amino acid residues (e.g., amino acids 21 to 282 of SEQ ID NO: 8 or amino acids 1 to 261 of SEQ ID NO: 9), or at least 276 amino acid residues (e.g., amino acids 21 to 297 of SEQ ID NO: 8 or amino acids 1 to 276 of SEQ ID NO: 9), and wherein the fragment has ferulic acid esterase and/or acetyl xylan esterase activity;
(d) a fragment of SEQ ID NO: 11 or 12, wherein the fragment preferably contains at least 234 amino acid residues (e.g., amino acids 19 to 253 of SEQ ID NO: 11 or amino acids 1 to 234 of SEQ ID NO: 12), at 255 amino acid residues (e.g., amino acids 19 to 274 of SEQ ID NO: 11 or amino acids 1 to 255 of SEQ ID NO: 12), or at least 270 amino acid residues (e.g., amino acids 19 to 289 of SEQ ID NO: 11 or amino acids 1 to 270 of SEQ ID NO: 12), and wherein the fragment has ferulic acid esterase and/or acetyl xylan esterase activity; or
(e) a fragment of SEQ ID NO: 14 or 15, wherein the fragment preferably contains at least 227 amino acid residues (e.g., amino acids 19 to 253 of SEQ ID NO: 14 or amino acids 1 to 227 of SEQ ID NO: 15), at 240 amino acid residues (e.g., amino acids 20 to 260 of SEQ ID NO: 14 or amino acids 1 to 240 of SEQ ID NO: 15), or at least 254 amino acid residues (e.g., amino acids 20 to 274 of SEQ ID NO: 14 or amino acids 1 to 254 of SEQ ID NO: 15), and wherein the fragment has ferulic acid esterase and/or acetyl xylan esterase activity.

7. The polypeptide of any one of paragraphs 1-6, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 2, SEQ ID NO: 5, SEQ ID NO: 8, SEQ ID NO: 11, or SEQ ID NO: 14.

8. The polypeptide of any one of paragraphs 1-7, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 3, SEQ ID NO: 6, SEQ ID NO: 9, SEQ ID NO: 12, or SEQ ID NO: 15.

9. The polypeptide of any one of paragraphs 1-8, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to a mature polypeptide of SEQ ID NO: 2, SEQ ID NO: 5, SEQ ID NO: 8, SEQ ID NO: 11, or SEQ ID NO: 14.

10. The polypeptide of any one of paragraphs 1-9, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1, SEQ ID NO: 4, SEQ ID NO: 7, SEQ ID NO: 10 or SEQ ID NO: 13.

11. The polypeptide of any one of paragraphs 1-10, which is:
(a) a fragment of SEQ ID NO: 2, a fragment of a mature polypeptide of SEQ ID NO: 2, or a fragment of SEQ ID NO: 3, wherein the fragment preferably contains at least 235 amino acid residues (e.g., amino acids 20 to 255 of SEQ ID NO: 2 or amino acids 1 to 235 of SEQ ID NO: 3), at least 248 amino acid residues (e.g., amino acids 20 to 268 of SEQ ID NO: 2 or amino acids 1 to 248 of SEQ ID NO: 3), or at least 262 amino acid residues (e.g., amino acids 20 to 282 of SEQ ID NO: 2 or amino acids 1 to 262 of SEQ ID NO: 3), and wherein the fragment has ferulic acid esterase and/or acetyl xylan esterase activity;
(b) a fragment of SEQ ID NO: 5, a fragment of a mature polypeptide of SEQ ID NO: 5, or a fragment of SEQ ID NO: 6, wherein the fragment preferably contains at least 219 amino acid residues (e.g., amino acids 25 to 244 of SEQ ID NO: 5 or amino acids 1 to 219 of SEQ ID NO: 6), at least 232 amino acid residues (e.g., amino acids 25 to 257 of SEQ ID NO: 5 or amino acids 1 to 232 of SEQ ID NO: 6), or at least 245 amino acid residues (e.g., amino acids 25 to 270 of SEQ ID NO: 5 or amino acids 1 to 245 of SEQ ID NO: 6), and wherein the fragment has ferulic acid esterase and/or acetyl xylan esterase activity;
(c) a fragment of SEQ ID NO: 8, a fragment of a mature polypeptide of SEQ ID NO: 8, or a fragment of SEQ ID NO: 9, wherein the fragment preferably contains (e.g., amino acids 21 to 268 of SEQ ID NO: 8 or amino acids 1 to 247 of SEQ ID NO: 9), at least 261 amino acid residues (e.g., amino acids 21 to 282 of SEQ ID NO: 8 or amino acids 1 to 261 of SEQ ID NO: 9), or at least 276 amino acid residues (e.g., amino acids 21 to 297 of SEQ ID NO: 8 or amino acids 1 to 276 of SEQ ID NO: 9), and wherein the fragment has ferulic acid esterase and/or acetyl xylan esterase activity;
(d) a fragment of SEQ ID NO: 11, a fragment of a mature polypeptide of SEQ ID NO: 11, or a fragment of SEQ ID NO: 12, wherein the fragment preferably contains at least 234 amino acid residues (e.g., amino acids 19 to 253 of SEQ ID NO: 11 or amino acids 1 to 234 of SEQ ID NO: 12), at 255 amino acid residues (e.g., amino acids 19 to 274 of SEQ ID NO: 11 or amino acids 1 to 255 of SEQ ID NO: 12), or at least 270 amino acid residues (e.g., amino acids 19 to 289 of SEQ ID NO: 11 or amino acids 1 to 270 of SEQ ID NO: 12), and wherein the fragment has ferulic acid esterase and/or acetyl xylan esterase activity; or
(e) a fragment of SEQ ID NO: 14, a fragment of a mature polypeptide of SEQ ID NO: 14, or a fragment of SEQ ID NO: 15, wherein the fragment preferably contains at least 227 amino acid residues (e.g., amino acids 19 to 253 of SEQ ID NO: 14 or amino acids 1 to 227 of SEQ ID NO: 15), at 240 amino acid residues (e.g., amino acids 20 to 260 of SEQ ID NO: 14 or amino acids 1 to 240 of SEQ ID NO: 15), or at least 254 amino acid residues (e.g., amino acids 20 to 274 of SEQ ID NO: 14 or amino acids 1 to 254 of SEQ ID NO: 15), and wherein the fragment has ferulic acid esterase and/or acetyl xylan esterase activity.

12. The polypeptide of any one of paragraphs 1-11, which is:
(a) a variant of SEQ ID NO: 2, a variant of a mature polypeptide of SEQ ID NO: 2, or a variant of SEQ ID NO: 3 comprising a substitution, deletion, and/or insertion at one or more positions;
(b) a variant of SEQ ID NO: 5, a variant of a mature polypeptide of SEQ ID NO: 5, or a variant of SEQ ID NO: 6 comprising a substitution, deletion, and/or insertion at one or more positions;
(c) a variant of SEQ ID NO: 8, a variant of a mature polypeptide of SEQ ID NO: 8, or a variant of SEQ ID NO: 9 comprising a substitution, deletion, and/or insertion at one or more positions;
(d) a variant of SEQ ID NO: 11, a variant of a mature polypeptide of SEQ ID NO: 11, or a variant of SEQ ID NO: 12 comprising a substitution, deletion, and/or insertion at one or more positions; or
(e) a variant of SEQ ID NO: 14, a variant of a mature polypeptide of SEQ ID NO: 14, or a variant of SEQ ID NO: 15 comprising a substitution, deletion, and/or insertion at one or more positions.

13. The polypeptide of any one of paragraphs 1-10, comprising, consisting essentially of, or consisting of SEQ ID NO: 2, SEQ ID NO: 5, SEQ ID NO: 8, SEQ ID NO: 11, or SEQ ID NO: 14.

14. The polypeptide of any one of paragraphs 1-10, comprising, consisting essentially of, or consisting of a mature polypeptide of SEQ ID NO: 2, SEQ ID NO: 5, SEQ ID NO: 8, SEQ ID NO: 11, or SEQ ID NO: 14.

15. The polypeptide of any one of paragraphs 1-10, comprising, consisting essentially of, or consisting of SEQ ID NO: 3, SEQ ID NO: 6, SEQ ID NO: 9, SEQ ID NO: 12, or SEQ ID NO: 15.

16. The polypeptide of any one of paragraphs 1-15, comprising an N-terminal extension and/or C-terminal extension of 1-10 amino acids, e.g., 1, 2, 3, 4, 5, 6, 7, 8, 9, or 10 amino acids, preferably an extension of 1-5 amino acid residues in the N-terminus and/or 1-5 amino acids in the C-terminus, such as 1 amino acid residue and wherein the extended polypeptide has ferulic acid activity.

17. The polypeptide of any one of paragraphs 1-16 having:
(a) at most 10%, at most 9%, at most 8%, at most 7%, at most 6%, at most 5%, at most 4%, at most 3%, at most 2% or at most 1% sequence differences to the polypeptide of SEQ ID NO: 2, a mature polypeptide of SEQ ID NO: 2 or the polypeptide of SEQ ID NO: 3;
(b) at most 10%, at most 9%, at most 8%, at most 7%, at most 6%, at most 5%, at most 4%, at most 3%, at most 2% or at most 1% sequence differences to the polypeptide of SEQ ID NO: 5, a mature polypeptide of SEQ ID NO: 5 or the polypeptide of SEQ ID NO: 6;
(c) at most 10%, at most 9%, at most 8%, at most 7%, at most 6%, at most 5%, at most 4%, at most 3%, at most 2% or at most 1% sequence differences to the polypeptide of SEQ ID NO: 8, a mature polypeptide of SEQ ID NO: 8 or the polypeptide of SEQ ID NO: 9;
(d) at most 10%, at most 9%, at most 8%, at most 7%, at most 6%, at most 5%, at most 4%, at most 3%, at most 2% or at most 1% sequence differences to the polypeptide of SEQ ID NO: 11, a mature polypeptide of SEQ ID NO: 11 or the polypeptide of SEQ ID NO: 12; or
(e) at most 10%, at most 9%, at most 8%, at most 7%, at most 6%, at most 5%, at most 4%, at most 3%, at most 2% or at most 1% sequence differences to the polypeptide of SEQ ID NO: 14, a mature polypeptide of SEQ ID NO: 14 or the polypeptide of SEQ ID NO: 15.

18. The polypeptide of any one of paragraphs 1-17, which differs from the polypeptide of SEQ ID NO:2, SEQ ID NO: 5, SEQ ID NO: 8, SEQ ID NO: 11, SEQ ID NO: 14, a mature polypeptide of SEQ ID NO: 2, SEQ ID NO: 5, SEQ ID NO: 8, SEQ ID NO: 11, SEQ ID NO: 14, or the polypeptide of SEQ ID NO: 3, SEQ ID NO: 6, SEQ ID NO: 9, SEQ ID NO: 12 or SEQ ID NO: 15 by at most 20 amino acids, such as at most 1, 2, 3, 4, 5, 6, 7, 8, 9, 10, 11, 12, 13, 14 or 15 amino acids.

19. The polypeptide of any one of paragraphs 1-18, which is obtained from or obtainable from a fungus of the genus Microsphaeropsis, such as a Microsphaeropsis amaranthi fungus, a Microsphaeropsis arundinis fungus, a Microsphaeropsis fusca fungus, a Microsphaeropsis hellebore fungus, a Microsphaeropsis olivacea fungus, a Microsphaeropsis ononidicola fungus, a Microsphaeropsis proteae fungus, a Microsphaeropsis spartii-uncei fungus.

20. The polypeptide of any one of paragraphs 1-19, which has ferulic acid esterase and/or acetyl xylan esterase activity.

21. The polypeptide of any one of paragraphs 1-20, which is isolated.

22. The polypeptide of any one of paragraphs 1-21, which is purified.

23. A fusion polypeptide comprising the polypeptide of any one of paragraphs 1-22 and a second polypeptide.

24. The polypeptide of any one of paragraphs 1-23, wherein sequence identity is determined as the output of the longest identity using the Needleman-Wunsch algorithm.

25. The polypeptide of any one of paragraphs 1-23, wherein sequence identity is determined as the output of the longest identity using the Needleman-Wunsch algorithm as implemented in the Needle program of the EMBOSS package.

26. The polypeptide of paragraphs 24 or 25, wherein the parameters used are a gap open penalty of 10, a gap extension penalty of 0.5, and the EBLOSUM62 substitution matrix.

27. A granule, which comprises:
(a) a core comprising the polypeptide of any one of paragraphs 1-26 and optionally,
(b) a coating consisting of one or more layer(s) surrounding the core.

28. A granule, which comprises:
(a) a core, and
(b) a coating consisting of one or more layer(s) surrounding the core, wherein the coating comprises the polypeptide of any one of paragraphs 1-26.

29. A liquid composition comprising the polypeptide of any one of paragraphs 1-26 and an enzyme stabilizer, e.g., a polyol such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

30. The liquid composition of paragraph 29, further comprising a filler or carrier material.

31. The liquid composition of paragraph 29 or 30, further comprising a preservative.

32. A composition comprising the polypeptide or fusion polypeptide of any one of paragraphs 1-26, the granule of paragraph 27 or 28, or the liquid composition of any one of paragraphs 29-31.

33. The composition of paragraph 32, which is a liquid composition, solid composition, solution, dispersion, paste, powder, granule, granulate, coated granulate, tablet, cake, crystal, crystal slurry, gel or pellet.

34. A composition comprising a polypeptide having arabinofuranosidase activity on disubstituted arabinose, a polypeptide having arabinofuranosidase activity on monosubstituted arabinose, a xylanase, a beta-xylosidase and a carbohydrate esterase family 1 (CE1) polypeptide having ferulic acid esterase and/or acetyl xylan esterase activity, and optionally a GH31 alpha-xylosidase.

35. The composition of paragraph 34, wherein the polypeptide having arabinofuranosidase activity on disubstituted arabinose is a GH43 arabinofuranosidase.

36. The composition of paragraphs 34 or 35, wherein the GH43 arabinofuranosidase is a GH43_36 arabinofuranosidase.

37. The composition of any one of paragraphs 34-36, wherein the GH43 arabinofuranosidase is from the genus Humicola, Lasiodiplodia, or Poronia.

38. The composition of any one of paragraphs 34-37, wherein the GH43 arabinofuranosidase is from the species Humicola insolens, Lasiodiplodia theobromae, or Poronia punctata.

39. The composition of any one of paragraphs 34-38, wherein the GH43 arabinofuranosidase has an amino acid sequence selected from the group consisting of:
(i) the amino acid sequence of SEQ ID NO: 16 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 16, which has arabinofuranosidase activity;
(ii) the amino acid sequence of SEQ ID NO: 17 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 17, which has arabinofuranosidase activity; and
(iii) the amino acid sequence of SEQ ID NO: 18 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 18, which has arabinofuranosidase activity.

40. The composition of any one of paragraphs 34-39, wherein the polypeptide having arabinofuranosidase activity on monosubstituted arabinose is a GH51 arabinofuranosidase.

41. The composition of any one of paragraphs 34-40, wherein the GH51 arabinofuranosidase is a GH51_6 arabinofuranosidase.

42. The composition of any one of paragraphs 34-41, wherein the GH51 arabinofuranosidase has an amino acid sequence selected from the group consisting of:
(i) the amino acid sequence of SEQ ID NO: 19 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence to the amino acid sequence of SEQ ID NO: 19, which has arabinofuranosidase activity;
(ii) the amino acid sequence of SEQ ID NO: 20 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 20, which has arabinofuranosidase activity; and
(iii) the amino acid sequence of SEQ ID NO: 21 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 21, which has arabinofuranosidase activity.

43. The composition of any one of paragraphs 34-42, wherein the polypeptide having xylanase activity is a GH5_21 xylanase.

44. The composition of any one of paragraphs 34-43, wherein the GH5_21 xylanase is from the genus Bacteroides, Belliella, Chryseobacterium, or Sphingobacterium.

45. The composition of any one of paragraphs 34-44, wherein the GH5_21 xylanase is from the species Bacteroides cellulosilyticus CL02Y12C19, Belliella sp-64282, Chryseobacterium sp., Chryseobacterium oncorhynchi, or Sphingobacterium sp-64162.

46. The composition of any one of paragraphs 34-45, wherein the GH5_21 xylanase is from bioreactor metagenome, Elephant dung metagenome, Xanthan alkaline community O, Xanthan alkaline community S, or Xanthan alkaline community T.

47. The composition of any one of paragraphs 34-46, wherein the GH5_21 xylanase has an amino acid sequence selected from the group consisting of:
(i) the amino acid sequence of SEQ ID NO: 22 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 22, which has xylanase activity;
(ii) the amino acid sequence of SEQ ID NO: 23 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 23, which has xylanase activity;
(iii) the amino acid sequence of SEQ ID NO: 24 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 24, which has xylanase activity;
(iv) the amino acid sequence of SEQ ID NO: 25 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 25, which has xylanase activity;
(v) the amino acid sequence of SEQ ID NO: 26 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 26, which has xylanase activity;
(vi) the amino acid sequence of SEQ ID NO: 27 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 27, which has xylanase activity;
(vii) the amino acid sequence of SEQ ID NO: 28 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 28, which has xylanase activity;
(viii) the amino acid sequence of SEQ ID NO: 29 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 29, which has xylanase activity;
(ix) the amino acid sequence of SEQ ID NO: 30 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 30, which has xylanase activity;
(x) the amino acid sequence of SEQ ID NO: 31 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 31, which has xylanase activity;
(xi) the amino acid sequence of SEQ ID NO: 32 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 32, which has xylanase activity;
(xii) the amino acid sequence of SEQ ID NO: 33 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 33, which has xylanase activity;
(xiii) the amino acid sequence of SEQ ID NO: 34 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 34, which has xylanase activity;
(xiv) the amino acid sequence of SEQ ID NO: 35 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 35, which has xylanase activity; and
(xv) the amino acid sequence of SEQ ID NO: 36 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 36, which has xylanase activity.

48. The composition of any one of paragraphs 34-47, where in the polypeptide having xylanase activity is a GH5_35 xylanase.

49. The composition of any one of paragraphs 34-48, wherein the GH5_35 xylanase is from the genus Bacillus, Cohnella, or Paenibacillus.

50. The composition of any one of paragraphs 34-49, wherein the GH5_35 xylanase is from the species Bacillus hemiccellulosilyticus JCM 9152, Cohnella xylanilytica, Paenibacillus chitinolyticus, or Paenibacillus sp-62332.

51. The composition of any one of paragraphs 34-50, wherein the GH5_35 xylanase is from compost metagenome.

52. The composition of any one of paragraphs 34-51, wherein the GH5_35 xylanase has an amino acid sequence selected from the group consisting of:
(i) the amino acid sequence of SEQ ID NO: 37 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 37, which has xylanase activity;
(ii) the amino acid sequence of SEQ ID NO: 38 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 38, which has xylanase activity;
(iii) the amino acid sequence of SEQ ID NO: 39 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 39, which has xylanase activity;
(iv) the amino acid sequence of SEQ ID NO: 40 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 40, which has xylanase activity; and
(v) the amino acid sequence of SEQ ID NO: 41 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence to the amino acid sequence of SEQ ID NO: 41, which has xylanase activity.

53. The composition of any one of paragraphs 34-52, wherein the polypeptide having xylanase activity is a GH30_8 xylanase.

54. The composition of any one of paragraphs 34-53, wherein the GH30_8 xylanase is from the genus Bacillus.

55. The composition of any one of paragraphs 34-54, wherein the GH30_8 xylanase is from the species Bacillus sp-18423.

56. The composition of any one of paragraphs 34-55, wherein the GH30_8 xylanase has the amino acid sequence of SEQ ID NO: 42 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 42, which has xylanase activity.

57. The composition of any one of paragraphs 34-56, wherein the beta-xylosidase is a GH3 beta-xylosidase.

58. The composition of any one of paragraphs 34-57, wherein the GH3 beta-xylosidase is from the genus Aspergillus or Talaromyces.

59. The composition of any one of paragraphs 34-58, wherein the GH3 beta-xylosidase is from the species Aspergillus fumigatus, Aspergillus nidulans, or Talaromyces emersonii.

60. The composition of any one of paragraphs 34-59, wherein the GH3 beta-xylosidase has an amino acid sequence selected from the group consisting of:
(i) the amino acid sequence of SEQ ID NO: 43 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 43, which has beta-xylosidase activity;
(ii) the amino acid sequence of SEQ ID NO: 44 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 44, which has beta-xylosidase activity; and
(iii) the amino acid sequence of SEQ ID NO: 45 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 45, which has beta-xylosidase activity.

61. The composition of any one of paragraphs 34-60, wherein the CE1 polypeptide having ferulic acid esterase and/or acetyl xylan esterase activity is the polypeptide of any one of paragraphs 1-26.

62. The composition of any one of paragraphs 34-61, which comprises the GH31 alpha-xylosidase.

63. The composition of any one of paragraphs 34-62, wherein the GH31 alpha-xylosidase is from the genus Herbinix.

64. The composition of any one of paragraphs 34-63, wherein the GH31 alpha-xylosidase is from the species Herbinix hemicellulosilytica.

65. The composition of any one of paragraphs 34-64, wherein the GH31 alpha-xylosidase has the amino acid sequence of SEQ ID NO: 46 with from 0 to 10 conservative amino acid substitutions or one having at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 46, which has alpha-xylosidase activity.

66. A process for producing a fermentation product from a starch-containing material comprising the steps of:
(a) saccharifying the starch-containing material with an alpha-amylase and a glucoamylase at a temperature below the initial gelatinization temperature to produce a fermentable sugar;
(b) fermenting the sugar with a fermenting organism to produce the fermentation product;
wherein a CE1 polypeptide having ferulic acid esterase and/or acetyl xylan esterase activity or a composition comprising a CE1 polypeptide having ferulic acid esterase and/or acetyl xylan esterase activity is present or added during saccharifying step (a) and/or fermenting step (b).

67. The process of paragraph 66, wherein the CE1 polypeptide having ferulic acid esterase and/or acetyl xylan esterase activity of any one of paragraphs 1-26 is present or added during saccharifying step (a) and/or fermenting step (b).

68. The process of paragraph 66 or 67, wherein the composition of any one of paragraphs 34-65 is present or added during saccharifying step (a) and/or fermenting step (b).

69. The process of any one of paragraphs 66-68, wherein saccharifying step (a) and fermenting step (b) are performed simultaneously.

70. A process for producing a fermentation product from a starch-containing material comprising the steps of:
(a) liquefying a starch-containing material at a temperature above the initial gelatinization temperature of the starch with a thermostable alpha-amylase to produce a dextrin;
(b) saccharifying the dextrin with a glucoamylase to produce a fermentable sugar;
(c) fermenting the sugar with a fermenting organism to produce the fermentation product;
wherein a CE1 polypeptide having ferulic acid esterase and/or acetyl xylan esterase activity or a composition comprising a CE1 polypeptide having ferulic acid esterase and/or acetyl xylan esterase activity is present or added during saccharifying step (b) and/or fermenting step (c).

71. The process of paragraph 70, wherein the CE1 polypeptide having ferulic acid esterase and/or acetyl xylan esterase activity of any one of paragraphs 1-26 is present or added during saccharifying step (b) and/or fermenting step (c).

72. The process of paragraph 70 or 71, wherein the composition of any one of paragraphs 34-65 is present or added during saccharifying step (b) and/or fermenting step (c).

73. The process of any one of paragraphs 70-72, wherein saccharifying step (b) and fermenting step (c) are performed simultaneously.

74. The process of any one of paragraphs 70-73, wherein the thermostable alpha-amylase has the amino acid sequence of SEQ ID NO: 50 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 50, which has alpha-amylase activity.

75. The process of any one of paragraphs 70-74, wherein the thermostable alpha-amylase has the amino acid sequence of SEQ ID NO: 51 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 51, which has alpha-amylase activity.

76. The process of any one of paragraphs 70-75, wherein a thermostable protease and/or a thermostable xylanase are added in liquefying step (a).

77. The process of any one of paragraphs 70-76, wherein the thermostable protease has the amino acid sequence of SEQ ID NO: 52 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 52, which has protease activity.

78. The process of any one of paragraphs 70-77, wherein the thermostable xylanase has an amino acid sequence of SEQ ID NO: 53 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 53, which has xylanase activity.

79. The process of any one of paragraphs 66 to 78, wherein the glucoamylase has an amino acid sequence of SEQ ID NO: 54 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 54, which has glucoamylase activity.

80. The process of any one of paragraphs 70 to 79, further comprising adding an alpha-amylase during saccharifying step (b) and/or fermenting step (c).

81. The process of any one of paragraphs 66 to 80, wherein the alpha-amylase has an amino acid sequence of SEQ ID NO: 55 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 55, which has alpha-amylase activity.

82. The process of any one of paragraphs 70 to 81, wherein a trehalase is added during the saccharifying step and/or the fermenting step.

83. The process of paragraph 82, wherein the trehalase has an amino acid sequence of SEQ ID NO: 56 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 56, which has trehalase activity.

84. The process of any one of paragraphs 70 to 83, wherein a composition comprising a beta-glucosidase, a cellobiohydrolase, and an endoglucanase are added during the saccharifying step and/or the fermenting step.

85. The process of paragraph 84, wherein the beta-glucosidase has an amino acid sequence of SEQ ID NO: 57 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 57, which has beta-glucosidase activity.

86. The process of paragraphs 84-85, wherein the cellobiohydrolase has an amino acid sequence of SEQ ID NO: 58 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 58, which has cellobiohydrolase activity.

87. The process of any one of paragraphs 84-86, wherein the endoglucanase has an amino acid sequence of SEQ ID NO: 59 with from 0 to 10 conservative amino acid substitutions or an amino acid sequence that is at least 60%, at least 65%, at least 70%, at least 75%, at least 80%, at least 85%, at least 90%, at least 95%, at least 96%, at least 97%, at least 98%, or at least 99% sequence identity to the amino acid sequence of SEQ ID NO: 59, which has endoglucanase activity.

88. The process of any one of paragraphs 66-87, wherein the starch-containing material comprises beets, maize, corn, wheat, rye, oats, barley, triticale, rice, sweet potatoes, sorghum, millet, pearl millet, and/or foxtail millet.

89. The process of any one of paragraphs 66-88, wherein the starch-containing material comprises corn.

90. The process of any one of paragraphs 66-88, wherein the fermentation product is ethanol, preferably fuel ethanol.

91. The process of any one of paragraphs 66-90, wherein the fermenting organism is yeast.

92. A polynucleotide encoding the polypeptide or fusion polypeptide of any one of paragraphs 1-26.

93. The polynucleotide of paragraph 92, which comprises:
(i) SEQ ID NO: 1 or nucleotides 60 to 885 of SEQ ID NO: 1;
(ii) SEQ ID NO: 4 or nucleotides 75 to 846 of SEQ ID NO: 4;
(iii) SEQ ID NO: 7 or nucleotides 63 to 830 of SEQ ID NO: 7;
(iv) SEQ ID NO: 10 or nucleotides 57 to 906 of SEQ ID NO: 10; and
(v) SEQ ID NO: 13 or nucleotides 60 to 858 of SEQ ID NO: 13.

94. The polynucleotide of paragraph 92 or 93, which is isolated.

95. The polynucleotide of any one of paragraphs 92-94, which is purified.

96. A nucleic acid construct or expression vector comprising the polynucleotide of any one of paragraphs 92-95, wherein the polynucleotide is operably linked to one or more control sequences that direct the production of the polypeptide in an expression host.

97. A recombinant host cell comprising the polynucleotide of any one of paragraphs 92-96 operably linked to one or more control sequences that direct the production of the polypeptide or the fusion polypeptide.

98. The recombinant host cell of paragraph 97, wherein the polypeptide is heterologous to the recombinant host cell.

99. The recombinant host cell of paragraph 97 or 98, wherein at least one of the one or more control sequences is heterologous to the polynucleotide encoding the polypeptide.

100. The recombinant host cell of any one of paragraphs 97-99, which comprises at least two copies, e.g., three, four, five, or more copies of the polynucleotide of any one of paragraphs 31-34.

101. The recombinant host cell of any one of paragraphs 97-100, which is a yeast recombinant host cell, e.g., a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

102. The recombinant host cell of any one of paragraphs 97-101, which is a filamentous fungal recombinant host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

103. The recombinant host cell of any one of paragraphs 97-102, which is a prokaryotic recombinant host cell, e.g., a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, or a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells.

104. The recombinant host cell of any one of paragraphs 97-103 which is isolated.

105. The recombinant host cell of any one of paragraphs 97-104, which is purified.

106. A method of producing the polypeptide of any one of paragraphs 1-26, comprising cultivating a cell, which in its wild-type form produces the polypeptide, under conditions conducive for production of the polypeptide.

107. The method of paragraph 106, further comprising recovering the polypeptide or the fusion polypeptide.

108. A method of producing a polypeptide having ferulic acid esterase and/or acetyl xylan esterase activity, comprising cultivating the recombinant host cell of any one of paragraphs 97-105 under conditions conducive for production of the polypeptide or the fusion polypeptide.

109. The method of paragraph 108, further comprising recovering the polypeptide or the fusion polypeptide.

110. A whole broth formulation or cell culture composition comprising the polypeptide of any one of paragraphs 1-26 or the fusion polypeptide of paragraph 23.

1. A polypeptide having ferulic acid esterase and/or acetyl xylan esterase activity, selected from the group consisting of:
(i)
(a) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 2;
(b) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 3;
(c) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 2;
(d) a polypeptide encoded by a polynucleotide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 1;
(e) a polypeptide derived from SEQ ID NO: 2, a mature polypeptide of SEQ ID NO: 2, or SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity;
(ii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 5;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 6;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 5;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 4;
(e) a polypeptide derived from SEQ ID NO: 5, a mature polypeptide of SEQ ID NO: 5, or SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 8;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 9;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 8;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 7;
(e) a polypeptide derived from SEQ ID NO: 8, a mature polypeptide of SEQ ID NO: 8, or SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iv)
(a) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 11;
(b) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 12;
(c) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 11;
(d) a polypeptide encoded by a polynucleotide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 10;
(e) a polypeptide derived from SEQ ID NO: 11, a mature polypeptide of SEQ ID NO: 11, or SEQ ID NO: 12 by having 1-30 alterations (e.g., substitutions, deletions and/or insertions atone or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity; and
(v)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 14;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 15;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 14;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 13;
(e) a polypeptide derived from SEQ ID NO: 14, a mature polypeptide of SEQ ID NO: 14, or SEQ ID NO: 15 by having 1-30 alterations (e.g., substitutions, deletions and/or insertions atone or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity.

2. (canceled)

3. (canceled)

4. (canceled)

5. A granule, which comprises:
(a) a core comprising the polypeptide of claim 1, and, optionally
(b) a coating consisting of one or more layer(s) surrounding the core.

6. A granule, which comprises:
(a) a core, and
(b) a coating consisting of one or more layer(s) surrounding the core, wherein the coating comprises the polypeptide of claim 1.

7. A composition comprising the polypeptide of claim 1.

8. A composition comprising a carbohydrate esterase family 1 (CE1) polypeptide having ferulic acid esterase and/or acetyl xylan esterase activity, a polypeptide having arabinofuranosidase activity on disubstituted arabinose, a polypeptide having arabinofuranosidase activity on monosubstituted arabinose, a xylanase, a beta-xylosidase and, and optionally an alpha-xylosidase.

9. (canceled)

10. (canceled)

11. (canceled)

12. (canceled)

13. (canceled)

14. (canceled)

15. (canceled)

16. The composition of claim 8, wherein the CE1 polypeptide having ferulic acid esterase and/or acetyl xylan esterase activity is selected from the group consisting of:
(a) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 2;
(b) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 3;
(c) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 2;
(d) a polypeptide encoded by a polynucleotide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 1;
(e) a polypeptide derived from SEQ ID NO: 2, a mature polypeptide of SEQ ID NO: 2, or SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity;
(ii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 5;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 6;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 5;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 4;
(e) a polypeptide derived from SEQ ID NO: 5, a mature polypeptide of SEQ ID NO: 5, or SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 8;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 9;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 8;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 7;
(e) a polypeptide derived from SEQ ID NO: 8, a mature polypeptide of SEQ ID NO: 8, or SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iv)
(a) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 11;
(b) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 12;
(c) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 11;
(d) a polypeptide encoded by a polynucleotide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 10;
(e) a polypeptide derived from SEQ ID NO: 11, a mature polypeptide of SEQ ID NO: 11, or SEQ ID NO: 12 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity; and
(v)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 14;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 15;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 14;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 13;
(e) a polypeptide derived from SEQ ID NO: 14, a mature polypeptide of SEQ ID NO: 14, or SEQ ID NO: 15 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity.

17. The composition of claim 7, wherein the composition comprises the alpha-xylosidase.

18. The composition of claim 7, wherein the alpha-xylosidase is a GH31 alpha-xylosidase.

19. A process for producing a fermentation product from a starch-containing material comprising the steps of:
(a) saccharifying a starch-containing material with a glucoamylase and an alpha-amylase at a temperature below the initial gelatinization temperature of the starch to produce a fermentable sugar;
(b) fermenting the sugar with a fermenting organism;
wherein a CE1polypeptide having ferulic acid esterase activity and/or acetyl xylan esterase activity or a composition comprising a CE1 polypeptide having ferulic acid esterase activity and/or acetyl xylan esterase activity is present or added during saccharifying step (a) and/or fermenting step (b).

20. The process of claim 19, wherein the CE1 polypeptide is selected from the group consisting of:
(i)
(a) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 2;
(b) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 3;
(c) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 2;
(d) a polypeptide encoded by a polynucleotide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 1;
(e) a polypeptide derived from SEQ ID NO: 2, a mature polypeptide of SEQ ID NO: 2, or SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity;
(ii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 5;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 6;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 5;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 4;
(e) a polypeptide derived from SEQ ID NO: 5, a mature polypeptide of SEQ ID NO: 5, or SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 8;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 9;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 8;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 7;
(e) a polypeptide derived from SEQ ID NO: 8, a mature polypeptide of SEQ ID NO: 8, or SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iv)
(a) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 11;
(b) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 12;
(c) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 11;
(d) a polypeptide encoded by a polynucleotide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 10;
(e) a polypeptide derived from SEQ ID NO: 11, a mature polypeptide of SEQ ID NO: 11, or SEQ ID NO: 12 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity; and
(v)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 14;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 15;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 14;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 13;
(e) a polypeptide derived from SEQ ID NO: 14, a mature polypeptide of SEQ ID NO: 14, or SEQ ID NO: 15 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity.

21. The process of claim 19, wherein the composition comprising the CE1 polypeptide comprises a carbohydrate esterase family 1 (CE1) polypeptide having ferulic acid esterase and/or acetyl xylan esterase activity, a polypeptide having arabinofuranosidase activity on disubstituted arabinose, a polypeptide having arabinofuranosidase activity on monosubstituted arabinose, a xylanase, a beta-xylosidase and, and optionally an alpha-xylosidase.

22. A process for producing a fermentation product from a starch-containing material comprising the steps of:
(a) liquefying a starch-containing material at a temperature above the initial gelatinization temperature of the starch with a thermostable alpha-amylase to produce a dextrin;
(b) saccharifying the dextrin with a glucoamylase to produce a fermentable sugar;
(c) fermenting the sugar with a fermenting organism to produce the fermentation product;
wherein a CE1 polypeptide having ferulic acid esterase activity and/or acetyl xylan esterase activity or a composition comprising the CE1 polypeptide having ferulic acid esterase activity and/or acetyl xylan esterase activity is present or added during saccharifying step (b) and/or fermenting step (c).

23. The process of claim 22, wherein the CE1 polypeptide is selected from the group consisting of:
(i)
(a) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 2;
(b) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 3;
(c) a polypeptide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 2;
(d) a polypeptide encoded by a polynucleotide having at least 83%, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 1;
(e) a polypeptide derived from SEQ ID NO: 2, a mature polypeptide of SEQ ID NO: 2, or SEQ ID NO: 3 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase and/or acetyl xylan esterase activity;
(ii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 5;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 6;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 5;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 4;
(e) a polypeptide derived from SEQ ID NO: 5, a mature polypeptide of SEQ ID NO: 5, or SEQ ID NO: 6 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iii)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 8;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 9;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 8;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 7;
(e) a polypeptide derived from SEQ ID NO: 8, a mature polypeptide of SEQ ID NO: 8, or SEQ ID NO: 9 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity;
(iv)
(a) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 11;
(b) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 12;
(c) a polypeptide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 11;
(d) a polypeptide encoded by a polynucleotide having at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 10;
(e) a polypeptide derived from SEQ ID NO: 11, a mature polypeptide of SEQ ID NO: 11, or SEQ ID NO: 12 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity; and
(v)
(a) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 14;
(b) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to SEQ ID NO: 15;
(c) a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to a mature polypeptide of SEQ ID NO: 14;
(d) a polypeptide encoded by a polynucleotide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% sequence identity to the mature polypeptide coding sequence of SEQ ID NO: 13;
(e) a polypeptide derived from SEQ ID NO: 14, a mature polypeptide of SEQ ID NO: 14, or SEQ ID NO: 15 by having 1-30 alterations (e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions);
(f) a polypeptide derived from the polypeptide of (a), (b), (c), (d), or (e), wherein the N- and/or C-terminal end has been extended by addition of one or more amino acids; and
(g) a fragment of the polypeptide of (a), (b), (c), (d), or (e);
wherein the polypeptide has ferulic acid esterase activity and/or acetyl xylan esterase activity.

24. The process of claim 22, wherein the composition comprising the CE1 polypeptide comprises a carbohydrate esterase family 1 (CE1) polypeptide having ferulic acid esterase and/or acetyl xylan esterase activity, a polypeptide having arabinofuranosidase activity on disubstituted arabinose, a polypeptide having arabinofuranosidase activity on monosubstituted arabinose, a xylanase, a beta-xylosidase and, and optionally an alpha-xylosidase.

25. A polynucleotide encoding the polypeptide of claim 1.

26. The polynucleotide of claim 25, which comprises:
(i) SEQ ID NO: 1 or nucleotides 60 to 885 of SEQ ID NO: 1;
(ii) SEQ ID NO: 4 or nucleotides 75 to 846 of SEQ ID NO: 4;
(iii) SEQ ID NO: 7 or nucleotides 63 to 830 of SEQ ID NO: 7;
(iv) SEQ ID NO: 10 or nucleotides 57 to 906 of SEQ ID NO: 10; and
(v) SEQ ID NO: 13 or nucleotides 60 to 858 of SEQ ID NO: 13.

27. A nucleic acid construct or expression vector comprising the polynucleotide of claim 25, operably linked to one or more control sequences that direct the production of the polypeptide in an expression host.

28. A recombinant host cell comprising the nucleic acid construct or expression vector of claim 27.

29. A method of producing a polypeptide having esterase activity, comprising cultivating the recombinant host cell of claim 28 under conditions conducive for production of the polypeptide.


SEQUENCE LISTING (59 sequences)

SEQ ID NO: 1 - DNA, 888 bp, Microsphaeropsis arundinis
  source 1..888: mol_type = genomic DNA
atgcagttgttgcagcatgtgttctccctctcgctctgtgcaggagcagcgctcggagccttgacccaggtcactggtttcggcaccaacccgtcgaacatccagatgcacatttccgtgcctgccaaggtcgcatccaaccctgccatcatcgtcgccctccacccttgtggaggtactgcccagcagtggtactcgggtacgaagttgccttcgtattcggaatccttgggattcattctcatctatccccagacaaccaagtattcgaactgttgggacgtgaacaacgcaggctcgttgacccatggaggcaacggtgacgcattgggcatcgtctcgatgatcaactatacattggataagtacaaaggtgacaaatccaaagtcttcgtcatgggaggatcctcgggagccatgatggccaacgtgatggtcggctcgtatcccgacgtcttcgaagcgggagcagcgtactccggagtggcattcgcatgtttcgcaggttccaaatcggaccctacccctttcggagcgaaccagacctgtgcccagggcttgtcgcatacacctgcggactgggcacgatacgtgcagaacgcctatcctggctacactggcaaacggcctcgtttcctcgtggcgcacggcctcgccgacacgttggtccgacctgccgcaggctacgaacagctcaagcagtggtcggcagtgctcggcgtcaccaacacagcaaacaagactggtgccggagtggacgagggttcgcagtacacacagatgatttacggagactccaacaagctcgtgggctatttcggccagggcgtgggacacattgcaccccctgtggaaccggtgttgttgaaattcttcggactcacttaa

SEQ ID NO: 2 - protein, 295 aa, Microsphaeropsis arundinis
MQLLQHVFSLSLCAGAALGALTQVTGFGTNPSNIQMHISVPAKVASNPAIIVALHPCGGTAQQWYSGTKLPSYSESLGFILIYPQTTKYSNCWDVNNAGSLTHGGNGDALGIVSMINYTLDKYKGDKSKVFVMGGSSGAMMANVMVGSYPDVFEAGAAYSGVAFACFAGSKSDPTPFGANQTCAQGLSHTPADWARYVQNAYPGYTGKRPRFLVAHGLADTLVRPAAGYEQLKQWSAVLGVTNTANKTGAGVDEGSQYTQMIYGDSNKLVGYFGQGVGHIAPPVEPVLLKFFGLT

SEQ ID NO: 3 - protein, 19 aa, Microsphaeropsis arundinis
MQLLQHVFSLSLCAGAALG

SEQ ID NO: 4 - DNA, 849 bp, Microsphaeropsis arundinis
  source 1..849: mol_type = genomic DNA
atgttcttcccgaaccatttcttcgcaatttccgccttggtcttgctcgccctcttcaacaacgtgcatgcagcgacagccggatgtggtaagacccctactttgacatccggtacgaagacagtgacggtcaacggtaagcagaggcgatggatcctcaggatcccggacggttacaacaagacgcagccttataggctcatcttcggtctccactggagggatggcgatatgacgtcggtcgatggcggatccgcaccctactacggcctcaaggccctcgccaaaaactccgcgatcttcgtcgcacccgacggattgaacaaaggatgggcaaaccagggaggtgaagacattacattcattgacacaatcaggcagcagatcgagaacgacctctgtgtgaacgagaagctccgcttctccctcggcttctcctacggaggagccatgtcgtactcgctcgcctgttcgagggcgaaggatttccgagcgattgcggtgctctcgggagcgctcttgtccggctgttcgggtggaaacgatccgatcgcattctacggtcagcacggcgtgcgagattccgtcttgcctatctccatgggacgagacattagggatcgattcgtgaagaacaacggctgtcagaacatgcaggccagggaacctgccaagaactcgcgtcagcacattatgaacgtctatcagggatgtaagtcggattatcccacccagtggaccgcattcgatgccgaccatgtggccctccctggcgacacgggtggcgatgcaggcgtgaactcgttcacacccgcagacgtgtggaaattcttctcgcagttccagtaa

SEQ ID NO: 5 - protein, 282 aa, Microsphaeropsis arundinis
MFFPNHFFAISALVLLALFNNVHAATAGCGKTPTLTSGTKTVTVNGKQRRWILRIPDGYNKTQPYRLIFGLHWRDGDMTSVDGGSAPYYGLKALAKNSAIFVAPDGLNKGWANQGGEDITFIDTIRQQIENDLCVNEKLRFSLGFSYGGAMSYSLACSRAKDFRAIAVLSGALLSGCSGGNDPIAFYGQHGVRDSVLPISMGRDIRDRFVKNNGCQNMQAREPAKNSRQHIMNVYQGCKSDYPTQWTAFDADHVALPGDTGGDAGVNSFTPADVWKFFSQFQ

SEQ ID NO: 6 - protein, 24 aa, Microsphaeropsis arundinis
MFFPNHFFAISALVLLALFNNVHA

SEQ ID NO: 7 - DNA, 933 bp, Microsphaeropsis arundinis
  source 1..933: mol_type = genomic DNA
atgttgtactcgacctacctcgcaggcctcctcttgtcctccgcagcctcggtcttgggtgcccctggattctatcaggactccttcgcgtccgaatccccctccgtctcgttgggccacgccgacgccttgttcaagagggccaactccgcaggttgtggcaagacgcccacgctcacctccggtgtcaagactatcaacgtcaagggcaagaaccgccagtatacgatccgagtgccttccggatatgataagtcgaagccgtacaaattgatcttcgccttccattgggtgggaggcacaatgggcgatgtctcgtcgggtggttccgatggtgcgctctgggcgtactacggtatgcagaagaaatccaacgaaggtgccatcctcgtcgcacctcagggtctcaacaacggctgggccaactcgggaggtgaggatatcgcattcgtggatgcaatgcgaaacgcaattgaggccgatctctgtgtcgatacgaccaagcgcttcgccacaggcttctcgtatggaggctccatgtcgttctcgatcgcgtgttcgagggcaaaggacttccgagccgtcgccgtgatctcgggtggacagttgtccggctgtgatggcggtaacgaccccatcgcatatctcggaatccatggcatctcggacggtacgctcaacattgccggaggccgtgcgctccgggataggttcgtgaaaaacaacggttgtacgtcccagtcccctaaggaacctgcaccgggttccaagacccacgtcacaacaaactactcgggttgtaagtccggttacccggtcacgtggctcgccttcgatggtggccatgcaccggcacctgtcgatggaggatcggattcgggagccaggtcgtacacaccggacgtcatctggggtttcttcaaccagttctaa

SEQ ID NO: 8 - protein, 310 aa, Microsphaeropsis arundinis
MLYSTYLAGLLLSSAASVLGAPGFYQDSFASESPSVSLGHADALFKRANSAGCGKTPTLTSGVKTINVKGKNRQYTIRVPSGYDKSKPYKLIFAFHWVGGTMGDVSSGGSDGALWAYYGMQKKSNEGAILVAPQGLNNGWANSGGEDIAFVDAMRNAIEADLCVDTTKRFATGFSYGGSMSFSIACSRAKDFRAVAVISGGQLSGCDGGNDPIAYLGIHGISDGTLNIAGGRALRDRFVKNNGCTSQSPKEPAPGSKTHVTTNYSGCKSGYPVTWLAFDGGHAPAPVDGGSDSGARSYTPDVIWGFFNQF

SEQ ID NO: 9 - protein, 20 aa, Microsphaeropsis arundinis
MLYSTYLAGLLLSSAASVLG

SEQ ID NO: 10 - DNA, 909 bp, Microsphaeropsis arundinis
  source 1..909: mol_type = genomic DNA
atgcatttctccactctcgtctccggtttgctcgcactctgtactgtccagggagcggtcgtcggcacagatcatcagctcatcaaaagggcctcgctcacccaggtgcagaacttcggcaacaacccttcgggcatcaaaatgttcatctacgtgcctcagaaattgcaggccaaacctcccgtggtcctcgtcctccacgcgtgtgcctggaccgcaaccaacttcttcgccacaacgaagtatggccagttggcagaccagcatggctacgtcctcatctacggccagacaccgacggacggagcgtgttgggatgtgtcctcgaagcagactctcacgcatgatggtggctccgattcgactggtctcgcaaacatggtgcggtacgccttgaagaagtataacggtgacgcatccaaggtgtacgtcacaggcgaatcctccggtgccatgatgacccaggtgatgtccgcagtctaccccgacatcttcgcagcctcctcggagttctcgggagaaccggcaggatgtttctacactggtaccgtccgaggctggaacggagattgtgccggtggtaagatccacaagacacctgcgcagtgggcagagcaggtccacgcgatgtaccctggatacaccggtcagtaccccaggatgcaggtcttccatggcgatgtggacaaaactctcaacatcgcatcgttcaacgaatcgattaaggaatggtccggtgtcttcggttactccggcaacccgacccaggcgctcaacaacaaccctggttccaaattgactaagtacatttacggtgatcgcctccagggtatttggggtcacggcttcggacacgtcgtccccaccaacgagaccgaagcattgaaatggttcggcatcacctcgtaa

SEQ ID NO: 11 - protein, 302 aa, Microsphaeropsis arundinis
MHFSTLVSGLLALCTVQGAVVGTDHQLIKRASLTQVQNFGNNPSGIKMFIYVPQKLQAKPPVVLVLHACAWTATNFFATTKYGQLADQHGYVLIYGQTPTDGACWDVSSKQTLTHDGGSDSTGLANMVRYALKKYNGDASKVYVTGESSGAMMTQVMSAVYPDIFAASSEFSGEPAGCFYTGTVRGWNGDCAGGKIHKTPAQWAEQVHAMYPGYTGQYPRMQVFHGDVDKTLNIASFNESIKEWSGVFGYSGNPTQALNNNPGSKLTKYIYGDRLQGIWGHGFGHVVPTNETEALKWFGITS

SEQ ID NO: 12 - protein, 18 aa, Microsphaeropsis arundinis
MHFSTLVSGLLALCTVQG

SEQ ID NO: 13 - DNA, 861 bp, Microsphaeropsis arundinis
  source 1..861: mol_type = genomic DNA
atgctcttgtcgaccctcttcctcggcttgacatccttgacgaccctcgcaactgcagccttgtcctccggctgtaacaaagcccctacggtgacatccggagtcaaaacaatctcggtcaacggccagtccaggcagttcacgatccgtgtccccgataactaccagaacaacaagggttacaaattggtctatggtttgcactgggtcggtggtcggatggaccaggtcgcaaacggtggcgacacaggatccaaaggttggaagtactatggcatgcagattctcgcgaacgagactgccattttcgtggcaccccagggcatcaacggcaactggggcaactccggtggctcggacgtcaaattcatcgacgccatgaacgactatatcgacaagggtctctgtgtggacactacccagcgcttctcgattggcttctcctacggtggatcgatgacttatgcggtcgcctgtgcgagggcgaaacagttcaggggagtggccgtgattgccggtggcgtcttgtcgggctgtgatggtggaaacgacccgatcgcctatttcggtatccacggaatccgagatggaactctcaacattgccggtggacgctccatgagggaccggttcgtcaagaacaacggatgtgcatccatgtccggtgcgaaggagccctcccagggttccaggacccatatcaccacagcagcgaccggctgtaaagcaggcttccccgtcaaatgggcagcacatgacggtggccacattcaggcagcagcagataaaccggcacccgaggagaacggtgcagcatcctgggtgggtcccgaagtgtggaacttctggaactcgctctaa

SEQ ID NO: 14 - protein, 286 aa, Microsphaeropsis arundinis
MLLSTLFLGLTSLTTLATAALSSGCNKAPTVTSGVKTISVNGQSRQFTIRVPDNYQNNKGYKLVYGLHWVGGRMDQVANGGDTGSKGWKYYGMQILANETAIFVAPQGINGNWGNSGGSDVKFIDAMNDYIDKGLCVDTTQRFSIGFSYGGSMTYAVACARAKQFRGVAVIAGGVLSGCDGGNDPIAYFGIHGIRDGTLNIAGGRSMRDRFVKNNGCASMSGAKEPSQGSRTHITTAATGCKAGFPVKWAAHDGGHIQAAADKPAPEENGAASWVGPEVWNFWNSL

SEQ ID NO: 15 - protein, 19 aa, Microsphaeropsis arundinis
MLLSTLFLGLTSLTTLATA

SEQ ID NO: 16 - protein, 540 aa, Humicola insolens
VPHAGNLPRQASTFTNPVLWEDHPDLEVFRVGSVFYYSSSTFAYSPGAPVLKSYDLVHWTPVTHSVPRLNFGSNYDLPSGTPGAYVKGIWASTLRYRRSNDRFYWYGCVEGRTYLWTSPGGNALANNGEVPPSAWNWQHTATIDNCYYDAGLLIDDDDTMYIAYGNPTINVAQLSPDGTRQVRVQQRVYAHPQGQTVEGARMYKIRGNYYILVTRPADAEYVLRSTTGSPFGPYEARTLVSRIQGPLANAGFAHQGGIVDAPDGTWHYVAFMDAYPGGRIPVVAPLRWTADGWPEVVTDSQGRWGTSYPIPVRGAKNATEGLASTDLDEFRGTRFSEHWEWNHNPDTSKFTLLGGNEGGLILRTATVTGDLFAARNTLTRRIAGPKASGIFRLDVRGMRDGDRAGAVLFRDRAAYIGVWKQGNEARIVMVDDLRLNEDGWRTASTGRVAANGPVIDTNAQQDIWLRIDADITPAFGTNTERTTTFYYSIDGGRTYTRLGPAFAMTNSWRYFTGYRFGVFNFSTKSLGGEVKVKGFKMNMI

SEQ ID NO: 17 - protein, 500 aa, Lasiodiplodia theobromane
DFTNPVLWEDLADLDIFRVDDTFYYSASTMAYSPGAPILRSYDLANWEFIGHSVPTLDFGDAYDLNGGQAYVQGIWASFCNYRPSNKLFYWGGCIRSDLKTHIYTASDPSGEWSKHAVIDNCYYDAGLFIDPDDDTMYVAYGSTNINVAQLSADGTTQVSNQVVYESPSNPGYIEGSRMYKINGRHYIFLTRPPDAEYVLMSNDGPFGPYEGRYLVDRVGAPVASAGAPHQGGIVDTPNGDWYYMSFIDAYPGGRMPVLAPITFDDEGWPTVELVNGAWGETYPSPNVPTPPREVESPVGIDTFSGSSLSHQWEWNHNPDNTKWSLDGGLRLQTATVTDDLYAARNTLTHRIIGPASSGTIVLDFSSMADGDRAGLSLFRDQSAWIGIVKDAGATRLAVWDDITMDSSWNTNSTGSEAATADISGTKVYLRVEADIAPAGTKQGVFSYSTDGETFTTLGPVFTMNTAWQYFIGYRFGIFNFATQALGGSVLVESFEMQLV

SEQ ID NO: 18 - protein, 512 aa, Poronia punctata
LPHEQENTSLAARQSSTFNNPVLWEDLADLDVFRVDDAYYYSSSTMHYSPGAPILRSYDLVNWEYVGHSVPTLDWGSKYNMEGGERAYVKGIWASSLRYRPSNGKWYWIGCIEFGKTYIYTSDSETGPFEKSATLDTCLYDCGILIDDDDTMYVAYGNTQISVAQLDADGLSIVKSQEVFSTPGDIGTLEGSRMYKRDGQYYILTTRPANQEWVLKSSDPFGNYDYKILADGLSPPVGGGGNPHQGGLVDTSEGDWYYMAFEDAYPGGRIPVLAPVTWGDDGFPAVTTVDGAWGQSYDMPLPEHEVPSTVGTDSFEGSSLAAAWEWNHNPDTNGFEVSDGLTLKTVSVTTDLYSARNTLTRRIHGPKSTATIVIDVSNMADGDVAGLSLLRDQSAWIGVRKSNGGYNVAMTNSITMDSNWATNNEGSVVDQADVSETTIYLRGTVDITPGSGRTGSFSYSTDGDNFTPLGDDLTLNSDWQFFMGYRWGIFNYATQATGGSVLVSSFDQTVSS

SEQ ID NO: 19 - protein, 627 aa, Meripilus giganteus
VTVTVNKNPSHTVPSTLYGLMFEDINHSGDGGLYAELLQNRAFQQVTPNTAAALAAWHPISNAKLAVIQDPSPVSNALPNSLQFSVPSGSSGRVGFTNEGFWGIKVDSTWTYKASLFFRFPTSSSFSGALTVGLQTNAGRVLAQNSTQIRGTTTKWTQINLELHPTASAPDVSNSFFVTIDGAAGAGQTINFAMFSLFPPTFKNRPNGLRADIAETLAEMGPSFFRFPGGNNLEGQTTATRWQWNATVGSLLDRPGRVGDWGYVNTDGLGLLEYLQFFEDTGMEPIMAVWAGYSLGGTSLAENQLAPYIQQAIDQINFVIGDPAKSAPAALRASLGHPEPFTLRFVEVGNEDFFAAGSYPYRWHDFVTALQAQFPQIRFIATTNAWNPVLSPVPQSYDVHVYQTPTWFYQNAFYYDGFQRNGTTYFEGEYAAISTNANDLFGTVADGRLAFPTVQSATGEAAFMTGLERNSDIVFAASYAPLLQHVNSTQWTPDLVSYDAGSVIKSTSFFAQKLFALNKGDQYLPSTLPTNGGTLHWSITRASSSGKTFIKIANAGSSAQSLTFQLTQFNSVSSTGTLQVLTGPETASNTPEAPQAIVPKTSTIGTGKTFTYNAPAFSVSVITVTTN

SEQ ID NO: 20 - protein, 698 aa, Lasiodiplodia theobromae
RIPRLPRQSSAAPFINATATNVSSVALSINTKDTSLRNTTAPYLYGLMFEDINHSGDGGIYAELLTNRAFQGSNIVPGAVDGLAGNVIISSENPVVPYGPVLTGWAPIGDVRLTLDVLHPLSEALPTVLQVDIPANATGEVGILNYGWWGMDVSPQTYNASFYVLANEPRYMNNQTTFTVSLRSNLTGETFVSEQIGPVKVSTFDYTQLNASIVNTVTAPNSNNTFALTMDASEVAGQTLYFGLFSLFPETFKDRPNGLRKDIAQAFYDMKPRFLRFPGGNNLEGVSVDKRWKWWKTIGPLKDRPGRPGNWNYFNTDGLGLLEYLEWCEDMEIEPVLGVYAGFSLDVWGQSGTDWPQERMPEVLQEAIDELEFCMGDTSTKWGAKRAEYGHPEPFNIKYVEIGNEDWFSTTYPRRFTFLYDGLKAAYPNITYISTAYNEHPDYTIDIPAGAYWDTHHYEEPSYFLRNFDFYDNWQTDTQNEDVGVLLGEFSVYQLDTPDGIVNFSNPADVHVFYPRMISAISEGVYALGGERNPNTVKLSSYAPSLQNRNWYNWTPNMISYDADPSKTVLSTSYWQQLLFAHYRGTQTLPVTNTEGDFNPVYWQSSIEEETGSIYLKVINAGNQTMPLSVTIDAAYSKVNGTIMQNDDINGFNFVNNRTAIMPRPLGANGTEVPAISSDGSFIWTVPIHSITVLQFDL

SEQ ID NO: 21 - protein, 676 aa, Acidiella bohemica
ATVSVTVANGGGNATSPYQYGLMFEDINNSGDGGVYAELIQNRAFQGNSIYPSTTDYWNALGGASIALKNLTNPLSSALPTSLQVSAGNSTSGIIGISNVGFWGFPVVAGWEYKGSFWVSGGLDGNLTVCLTSIDNTQYAEASVEVASSSEWTQYNYTFSPSVSAPNSNNTLNFTFAASDLKGSANFQLLSLFPPTYNNRPNGNRIDLMQAMDALKPKLFRAPGGNNVEGNAAPYWWNWTQTIGPLEDRPGYPGTWGYENTDGLGLIEYMEWADDLGMDVSLAVWDGHYLDGTVLAEDELQPYIQAALDELEFLMGDASTKWGAVRESLGHGPWNIPYVEIGNEDSLSGGQPSYSNYRFQAFYDAIHAAYPNITIIASFFDVGPVAPPFGAAGDFHEYAIPLQMSNQFTYFDNYTTQNPILIGEYAIVDYDRPNTTGPEWYTGAPRAFVPFWYGSVSEAIFLLGAERNSEKIISAMYAPSFQNLNRWQWIPDLIEYDAYPGHTTMSTSWHMIKLLSNNLITENLPITITEGKFGPAFFVAGRNDNTGSHMAKIANYNSTGSTTMNIKFDGVGSGAVGNLTYITAPMNATNPIGGSIVEYHSETVPASHTGMFSFQLPEYSVAILEVTAANAGGGQNYGNPGGRPGWKNWDQWGQQWGHGGQWGNGQGGWGGGHGGW

SEQ ID NO: 22 - protein, 606 aa, Bacteroides cellulosilyticus
AKLPVLHVEGRYLKNEKGEIINLHGFTQTYSPFFNNNAWGNYDVQACLRYNKSMVDGIVAAGWKFNFVRMHLDPYWSDDPSMQSVRYEGHERFSETRFRKYLEELFVPMAEYFISKGMYVVMRPPGVCPSDVPYQGIEIGDTYQQFLLKVWDIVSQHPKLKNNMDVMFELANEPVRIKGTDGTYGSSGDGHFKNLQLYFQAIVDKIRTNCHNIVWVPGLSYQSSYAGYAIHRIEGENIGFAVHCYPGWYGSDAEEDSGEGIGSSTGGGYEAFQRGWDAQVGPVVAFAPIMVTEIDWAPKKYDATWGKSITGTAGAEGFGANFKYIADNSGNVSWLFFTTRSHELAAFKDVPGEPGNYTFLNDPEACPWAMYHWFKEYADGIVVNGELEKLELMGQEGNLRLQMGGTNYLKVKAVYSDGTVRMVTAEATVNSSDTNVLKVEQAAKLVAVAPGEATVTVIYQSAAGVSKQLTLQVTVISPFSLTADVFNPSIWEKGTFDESTRTLVTGQYGFGGWQYDSGLDLSGYKTVTVELGNDNESGVSFRLFDKNNYWSEPATYDFGSSRKIVVELNNMKDKNGGKIDPSHLYIAGFWSTGGKPIVIANVSLAN

SEQ ID NO: 23 - protein, 400 aa, unidentified
  REGION 1..400: note = GH5_21 xylanase from Xanthan alkaline community S
EEIEEEFFEEEEEEVIEEENNLKLDIDWPLPTPTLKVVGRNLEDPCGNKVLLHGVALTPSPWFNGGHVGVWRWNNYDVQGCLAYNKGLMDKLTNDEEGWYLNFIRLHIDPYWSNTPGPPIPENDISRFDFERFVTAVDEVIVPLIDHARTRGMYVILRPPGVCPQRIDLDDEYHQYLMSIWNHVSQHPQLKNRDHVMFELANEPIHILGTNGEWGSDTQPHFDALKLYFQPMVDMIRANGSENIIWIPGSGYQSHYRGFPNNPIEDNNYGYAVHIYPGYWGMDNNDPTTFRLNWNRHIKPVADIAPIAITEIDWGPEQYMVWGKGGVTGSPGAYGFGANFKMLADESGNVSWNLLAPENLIDRGDLDGGIAYDADPEACAYPVFNWFREYAKTKIICSGP

SEQ ID NO: 24 - protein, 552 aa, Sphingobacterium sp-64162
EKTLTSESERSLKSNVSAQAVTSWPRPTPTLHVGGKYLKDPCDNNIVLHGVAITPSPWFNGCQYGANSGYCTWDNYNVQGALNYNKAVMDKLSSAADGWYLNYIRLHIDPYWTNDPGAPIPEDDISRFNYNRLVTYTDQVIVPLINHARSRGMYVILRPPGVCPHRIAVNDAYHTYLKTVWTFLSQHTALKNADNVMFELANEPVEILGTNGTWGMTGNEHFAALKNFFQPLVNIIRNNGANNVCWIPGTGWQSHYQGYVTNQITGGNIGYAVHIYPGYWGGVNTYQAFQNAWNTNVKPIADIAPIAITETDWAPQGYGTFGTGSTGTAGGNGFGANLKYIVDQSGNVSWNLLTPDDLLHKGDPNAGTAFNNDWEACAAPSKQWFQQYAAYNYPMSNCTVTSLVNNGIYEIEFQTDANKVLDLKSGEDANGAALRPWTRNGANAQRWVAIDAGNGYWRFVSKASASSRCIDLTSNSNVLGTAIRLWQNYGNDAQAWKVTAVANGYYKITSKVDATRGWDVPSCTMDGNSNLQLWDYYGTSCQLFKFKFIAMN

SEQ ID NO: 25 - protein, 553 aa, Sphingobacterium sp-64162
EEKTLTSESERSLKSNVSAQAVTSWPRPTPTLHVGGKYLKDPCDNNIVLHGVAITPSPWFNGCQYGANSGYCTWDNYNVQGALNYNKAVMDKLSSAADGWYLNYIRLHIDPYWTNDPGAPIPEDDISRFNYNRLVTYTDQVIVPLINHARSRGMYVILRPPGVCPHRIAVNDAYHTYLKTVWTFLSQHTALKNADNVMFELANEPVEILGTNGTWGMTGNEHFAALKNFFQPLVNIIRNNGANNVCWIPGTGWQSHYQGYVTNQITGGNIGYAVHIYPGYWGGVNTYQAFQNAWNTNVKPIADIAPIAITETDWAPQGYGTFGTGSTGTAGGNGFGANLKYIVDQSGNVSWNLLTPDDLLHKGDPNAGTAFNNDWEACAAPSKQWFQQYAAYNYPMSNCTVTSLVNNGIYEIEFQTDANKVLDLKSGEDANGAALRPWTRNGANAQRWVAIDAGNGYWRFVSKASASSRCIDLTSNSNVLGTAIRLWQNYGNDAQAWKVTAVANGYYKITSKVDATRGWDVPSCTMDGNSNLQLWDYYGTSCQLFKFKFIAMN

SEQ ID NO: 26 - protein, 397 aa, unidentified
  REGION 1..397: note = GH5_21 xylanase from Xanthan alkaline community O
MEDADDQIIDDDVAIIDEDKDNEKIEWPLATPLLKVQGRQLVDPCGNEVLLHGVAITPSPWFNGGHVGVWRWNNYDVAGCLNYNKGVMDKLTNEDEGWYLNMVRLHIDPYWSNTPGEPIPENDISRFSFDRFMTAVNDVIVPLVEHAKTRGMYIILRPPGVAPHQIAVGDEYFDYLMKIWDHLSQHPELKNKDHVMFELANEPIHILGTNGVYGSDSPAHFEALKLFFQPMVDKIRQNGSENILWIPGSGYQSHYRGFPDYPIEDDNYGYAVHIYPGYWGQENNDPITFRRNWNTHIKPVADIAPIAITEIDWGPAQYGVWGKGGVTGTAGGWGFGANFKMLADESGNVSWNLLSPENLIHDGHLDGDIAYGGDPEACAYPVHKWFKEYAIEKEICD

SEQ ID NO: 27 - protein, 625 aa, unidentified
  REGION 1..625: note = GH5_21 xylanase from bioreactor metagenome
DKTPITEEPPKENPTTPEEETTDEESALPVLTVVGRYLKNNKGEIVNLHGFAQTYSPYFNQNAWNNYDVSGCLSYNKRLIDQMQSAGWKFNFIRMHLDPYWSDDTSKESVRYEGHERFSSARFQKYLDELFIPMAKYAISKGLYIVMRPPGVAPEKIAVGDDYQKFLLTVWNIVSKHPDIKNNMDIMFELANEPIHILGPDGTYAGTGSGHFMNLKSYFQTITDKVRANGAKNIIWVPGLSYQSSYSGYATYPIEGENIGYAVHVYPGWYGSDAQSPSAELGGVMGGGYDGFQRGWDAQVMPVANFAPIMITEMDWAPEKYNSSWGKSITGEVGGTGFGANFKYIMDRTGNASWLLFTSPHLLAQFKNVAGTEGNYAFLNDPEACPWPVYHWFKEYAGEAPAEGDLNSLQVAGVTNSLSIRMGDSKNFIVKALYVDGTTRVVTSKSSVSSSNSAVLQVDNAGKMVAVKEGQAQVTISYTSAKGVTKQLVLTVNVITPFPLTATMFNPSIWEKGTFDEGTRTLVTGKYGFGGWEYANGLNLSAFKTLTIELGNDNQSSVSFRLFDKTSYWTDPATYDFGSSRKVVVDLQNMKDKNGLRIDPSHLYIIGFWSTGDKPIVIDKVLLDY

SEQ ID NO: 28 - protein, 394 aa, unidentified
  REGION 1..394: note = GH5_21 xylanase from Xanthan alkaline community T
DPEDIIEDEKPEQSEIPSDEELSWPIPISALQVQGRDLIDECGNKVLMHGVAITPSPWFNGGHVGEWRWNNYDVGACLDYNRAVMNTLSKGEDEWYLNMIRLHIDPYWTNVPGEPISESDISRFSFQRFVTAIEEVIIPLIDHAESRGMYVILRPPGVCPDRISVDDEYHQYLLRIWDHISQQPRLKNRKHLMFELANEPIHILGTNGLYGSDTQAHFDALKLFFQPMVDLIRSNGANNVLWIPGSGYQSHYRGFPNNPIEDDNYGYAVHIYPGYWGQDNNDPVTFRRNWNTHIKPVADIAPIAITEIDWGPEQYGVWGKGGITGTAGAYGFGANFKALADESGNVSWNLLMPENLIDRGNPNAGLAYGGDPDACAYPSQKWFKKYASEKIACN

SEQ ID NO: 29 - protein, 400 aa, unidentified; identical to SEQ ID NO: 23
  REGION 1..400: note = GH5_21 xylanase from Xanthan alkaline community S

SEQ ID NO: 30 - protein, 393 aa, Belliella sp-64282
EDVDEVQEEEAPIHEEFVQDFEYPLGTPILKVQGRQLLDPCGKEVLLHGVAITPSPWFNGGHVGVWRWNNYDVDGCLTYNKGVMDKLTNPEEGWFLNMVRLHIDPYWSNTLGAPIPENDISRFDFDRFVTAVDQVILPLIDHAKSRGMYIILRPPGVAPHQIAVGDEYFEYLLKIWDHISQHPQLKNRDHVMFELANEPVQILGTNGQYGSDTPAHFEALKLFFQPMVDKIRENGAQNILWIPGSGYQSHYRGFPDFPIEDDNYGYAVHIYPGYWGQQNNDPTVFRNNWNTHIKPVADIAPIAITEIDWGPEQYGVWGKGGVTGTAGGWGFGANFKVLADESGNVSWNLLSPENLFHDGHLDGGLAYNADPQACAYPVHKWFKEYAKQKVKCD

SEQ ID NO: 31 - protein, 527 aa, Chryseobacterium oncorhynchi
ATPMLHVGGRYLKDPCDNNVVLHGVAITPSPWFNGCQYGANSGYCTWDNYNVQGALNYNKAVMNKLSSAGDGWYLNYIRLHIDPYWTNDPGPAIPENDISRFNYNRLVTYTDQVIIPLINHARSLGMYVILRPPGVCPNRIAVNDAYHSYLKTVWTFLSQHPGLKNADNVMFELANEPVEILGTNGTWGTTGNEHFAALKNFFQPLVNIIRNNGANNVCWIPGTGWQSHYQGYVNNQITGGNIGYAVHIYPGYWGGVNNYQAFQNAWNINVKPIADIAPIAITETDWAPQGYGTFGIGTTGTAGGSGFGANLKYIVDQSGNVSWNVLAPDNLLHKGDPNAGTAYNNDWEACAAPVKQWFQQYASSNYPVGNCNTNSSLINNGIYEIEFQTDANKVLDLKSGEDANGAVLRPWTRNGASAQRWVAIDAGNGYWRFVSKASASNRCIDLASNSNTLGTSIRLWQNYNNDAQVWQVVAVSNGYYKILSKVDPTRGWDIPNCTMDGNSNLHLWDYYGTSCQLFKFKYIGMN

SEQ ID NO: 32 - protein, 400 aa, unidentified
  REGION 1..400: note = GH5_21 xylanase from Xanthan alkaline community T
EEIEEEFFEEEEEEVIEEENNLKLDIDWPLPTPTLKVVGRNLEDPCGNKVLLHGVALTPSPWFNGGHVGVWRWNNYDVQGCLAYNKGLMDKLTNDEEGWYLNFIRLHIDPYWSNTPGPPIPENDISRFDFERFVTAVDEVIVPLIDHAKTRGMYVILRPPGVCPQKIDLDDEYHQYLMSIWNHVSQHPQLKNRDHVMFELANEPIHILGTNGEWGSDTQPHFDALKLYFQPMVDMIRANGSENIIWIPGSGYQSHYRGFPNNPIEDNNYGYAVHIYPGYWGMDNNDPTTFRLNWNRHIKPVADIAPIAITEIDWGPEQYMVWGKGGVTGSPGAYGFGANFKMLADESGNVSWNLLAPENLIDRGDLDGGIAYDADPEACAYPVFNWFREYAKTKIICSGP

SEQ ID NO: 33 - protein, 553 aa, Sphingobacterium
EEKTLTSESERSLKSNVSAQAVTNWPRPTPTLHVGGKYLKDPCDNNIVLHGVAITPSPWFNGCQYGANSGYCTWDNYNVQGALNYNKAVIDKLTSAADGWYLNYIRLHIDPYWTNDPGAAIPENDISRFNYNRLVTYTNQVIIPLINHARSRGMYVILRPPGVCPHRIAVNDAYHTYLKTVWTFLSQHASLKNADNVMFELANEPVEILGTNGTWGMTGNEHFAALKNFFQPLVNIIRNNGANNVCWIPGTGWQSHYQGYVNNQITGGNIGYAVHIYPGYWGGVNTYQAFQNAWNTNVKPIADIAPIAITETDWAPQGYGTFGTGYTGTAGGNGFGANLKYIVDQSGNVSWNLLAPDNLLHKGDPNGGTAYNNDWEACAAPSKQWFQQYAASNYPMSNCNVTSLVNNGIYEIEFQTDANKVLDLKSGQDADGAVLRPWTRNGANAQRWVAIDAGNGYWRFVSKASASNRCIDLASNSNALGTSIRLWQNYSNDAQTWKVTTVANGYYKIVSKVDATRGWDIPSCTMDGNSNLQLWDYYGTSCQLFKFKFIAMN

SEQ ID NO: 34 - protein, 577 aa, unidentified
  REGION 1..577: note = GH5_21 xylanase from elephant dung metagenome
ETLSPLHVEGRWLCNENGEHVNLHGFGQTYSPWFNEQGNGWGWGYSVSDCMKYNKGLIDKIISAGWKMNWLRLHMDPHWSNTPGVSTSGENDISAFNESRFRTYLNSLFIPMAEYANQHGMYVVMRPPGVCPEKIEIGGAYYKYLLKVWGIVASNEKLKNNPGIMFELANEPINIKGTDGQYTSYSDGSNQNMTKFFQAIVDSIRSCGANNILWVPGLGYQSQYAGFVKYPIQGENIGYAVHCYPGWYGSDSEADGGSVEQGVVTKGAGYSEFKAGWDNQVGPVAKIAPILITEMDWAPKSYNCSWGKATTGVAGGVGFGANFKYIVDKTGNVSWMLFTGPEHLAKYVDSKADGKTFLTDPEACVRPIYRWFKEYTDPNWVFEDTLASQTMYFPQTDTWINPSIWEKGSFDATTGALVTGQYGFGGWQFGTGLDLSKWKYLVIKLKQTASSGGWSFRVFDENSYWSNCYQKSVGSSLRAIVPLNGMYKTIDGSVSNSSKVNPSHIYIAGFWSYGGTPLYIDKLYLTNQSDYSEEVGLEQVAQDDDVPIVMFDVMGRRVETPHRGGFYMSKDRKFIVR

SEQ ID NO: 35 - protein, 819 aa, unidentified
  REGION 1..819: note = GH5_21 xylanase from elephant dung metagenome
LHVNGNQFNDPYGNKVVLHGVMDTPSPYFNSWRWGYSCNDGAVSGCLNYFEKIFTAITDTKQGAYCNLFRLHLDPCWTNDPNKTATGSETGEANISRFSNDRLVKYLNTVYWPIAQKALNHGMYVIMRPPGVCPDNLRVGDAYQNYLKTVWNTVTKNQNVIKNSGVVMIELANEPIHIYNQYGQASATAMRDYFQPVIDVIRKNGYTGIILVPGTGWQSNYKDYDKYPVNDNNYGYAVHDYPGWYSTSDQSYDHNTAIRAFGDNVPVVKKKPIVITEVDWSPEKAGTGHYNEHGQWVTSNYGTWATASTTKWGYAYKAVLDYYDNISMTLSGTDTYIDVDTYLNSGKVTGAFGNIWEACGAACMYWYKLWAQKDYAHATGSSSSSTTTQPSTGGSTSTGSTTTGNGEFTNVDLSSSLIPGWSQGGVRPNGWACNDAGDYPASGEAGSGPRVMQFNGGGDFKYGFYFRQQDASKPGYIQYGTTSGYELALNSFGNYCLTFNCAAWAGSPYVKAEVITSSGNVIASTIVKCNLNLNKNTNASTAKSDQGYLSFYVLNKGNFSVKFTPCADANGGSGTWVEAVVGNINLRYMGNPLSFEKANYVNPGWKILDGGKIVETGDAGSGPRIFNFPAGGQFNYGLYIRSTSTELSENYAEFGSRWGYGLNFLPGRYTLTYNCAAWTGSPYVKCEVINENDQVVASSITNCSLNLNKNLNASTANAPQGSVSFTVSNTGSYKLRWTPVANANGDAGYWVEAVIGHIKITQNSGNKVAAYTDDNDEVTSVAEVENEANSEETFNLSGQRVENPTKGIYIKNGKKVIVR

SEQ ID NO: 36 - protein, 527 aa, Chryseobacterium sp
ATPMLHVGGRYLKDPCDNNVLLHGVAITPSPWFNGCQYGANSGYCTWDNYNVQGCLNYNKAVMNKLTSAADGWYLNYIRLHIDPYWTNDPGPSIPENDISRFNYNRLVTYTDQVIIPLINHARSRGMYVILRPPGVCPNRIAVNDAYHSYLKTVWTFLSQHPGLKNADNVMFELANEPVEILGTNGTWGQTGNEHFAALKNFFQPLVNIIRNNGANNVCWIPGTGWQSHYQGYPNNQITGGNIGYAVHIYPSYWGGLSNYQAFQNAWNINVKPIADIAPIAITETDWAPQGYGTFGIGTTGTAGGSGFGANLKYIVDQSGNVSWNVLAPDNLLHKGDPNGGTAYNNDWEACAAPVKQWFQQYAKSNYPVGNCNTTSSLVNNGIYEIEFQTDANKVVDLKSGEDANGAVLRPWTRNGAAAQRWVAIDAGNGYWRFVSKASATNRCITLASNSNTLGTSIVLWQNYGNDAQAWQVVAVSNGYYKILSKVDPTRGWDIPNCTMDGNSNLHLWDYYGTSCQLFKFKYIGMN

SEQ ID NO: 37 - protein, 536 aa, Cohnella xylanilytica
WTGMPMSKLRVSGNQLVNSSGQPVLLSGWHQPSGSYWTYQNSSYYLNRNGGNRHAATLEYLKEITDTFTTTSGKYGSSHGWYMNQVRLFIDREDMGDVAAGTYNFAGLQAVTQNVIIPYINYAKTKGVYVTLGLDFTILNNQATTQANLDKFNQIWGYLSSQPGIKSADNVMFELINEPVLSYANGGWGGNPAEPDFADHWNALKNFQNSIISTIRNNGADNVIWAAGLGWDQYYQLCATYPLTDPLNNVGYAVHWYPGYGAHDDYATLQQIWDNTIKPCADQYPINITETTWFKTLPGDSSYWELFNGSNEGFGKNTKAIFTAAGNVSIAVHMNGFLLEAGPRSSFADPTAGLKYDGNAARDGMARFLFEWYYERAQLHPWNGMWNGIQSGSTYKIVNRASGKVLEVPGGANTNALQLQQYTDNGATAQQWVVTDVGSYNNYYKLRSVSSSDGKVMDVRNGTKTNGEAIQLMSDFNNTAQQFRLFKLSNGYWSIINVNSNKAVEVAGSSTANNAPIQQNLYRGNLNQQWQLVKID

SEQ ID NO: 38 - protein, 536 aa, Bacillus hemicellulosilyticus JCM 9152
WTGMPMDKLHVSGNQLVNSSGQTVLLNGWHQPSGSYWTYQGSNYYLDRNGGNRHAANLEYLKDISDTFTDTSPKYGNDHGWYMNQIRLFIDREDMGDVAAGTYNFAGLQEVTQNVIIPYVEYAKTKGLYVTLGLDFTLLNDQATTQSNLDKFNEIWGYLASRPELRSADHVMFEIVNEPVLSYANGKWGGHPSDPDFVDHWNALRDFQNSIIATIRNQGADNVIWAAGLGWDQYYQLCASHPLTDPLNNTGYAVHWYPGYGANDDYATLQQQWDTNIKPCADHYPINITETTWYKWQPGDPEYWHLFDGTNEGFGKNTKAIFTDAGNVSIAVHMNGFLLEPGVRSTFADPTAGLKYDGDPARDAMARFIFEWYYERAQLYPWNGIWNGIHNGATYKLQNRASGKMIDVPGGQNNNGLQLQQWADNNATAQQWIIDDMGTYNNFYRLTSVSSSDNKVMDVRNGSSHNGEAIQLMSDFGNSAQQFRLIRLSNGYWSILNVNSNKAVEVTGSSSADGALLQQNMYRGDLHQQWDLIRIN

SEQ ID NO: 39 - protein, 536 aa, Paenibacillus sp-62332
WSGMPMSKLRVSGNQLVNSSNQPVLLSGWHQPTGSYWTYQNSNYYLNRNGGNRHAAILEYLKDITDTFTSTSPKYGNNHGWYMNQVRLFIDRQDMGDVAAGTYNFAGLQSVTQNVIIPYIDYARTKGLYVTLGLDFTLLNDEATTQSNLDKFNQIWGYLAAQPGIKSADNVMFEIINEPVKSYANGRWGGHPSDPDFVAFWNSLRTFQNAIISTIRNQGADNVIWAAGLGWDQYYQLCASHPLTDPLNNLGYAVHWYPGYGANDNNSVLQQQWNTNIKPCADHFPINITETTWFKWLPGDSSYWNLFNGSNEGFGKNTKAIFTAAGNVSIAVHMNGFLLEPGTRSSFADPTAGLKFDGNAARDGMARFIFEWYYERAQMYPWNGIWNGISSGTTYKIVNRHSGKVIDVPGGQNNNALQLQQWADLHTSAQRWVVDDMGSSTNVYRLRSVSSSDNKVMDVRNGTINNGEAIQLMQDFGNTAQRFRLIRLSNGYWSIININSNKAVEVSGSSTADGANLQQNPYRGDHHQQWQLIAVN

SEQ ID NO: 40 - protein, 536 aa, unidentified
  REGION 1..536: note = GH5_35 xylanase from compost metagenome
WSGMPMSKLHVSGKQLVNSSGQPVLLSGWHQPSGPYWTYQNSNYYLSQNGNNRHAAILAYLKDITDTFTDTSAKYGSTHGWYMNQVRLFIDREDMGDVAAGTYNFAGLKSVTQNVIIPYINYAKTKGLYVTLGLDFTLQDNKATTSSNLAKFNEIWGYLASQTGIKSADNVMFELINEPVLSDVNGVWGGNPSQSNFVDYWNSLRNFQNSIISTIRSKGADNVIWAAGLGWDQYYQLCASHPLTDPLNNLGYSVHWYPGYGANDNFSTLQQIWNTTIKPCADNYPINITETTWFKRKAGDSDYWNLFNGSNEGFGKNTKAIFTAAGNVSIAAHMNGFILNPGTRSSFADPTAGLQYDGDATRDGMARFLFEWYYERAQFNPWNGVWNGLTNGGTYKLVNRASGKVIDVPGGQNTNALQLQQYADNGATAQQWVITDQGTYNNFYKLTSVSSSDGKVMDVRNGTSNNGEAIQLMQSFNNTAQQFRLIKLSNGYYCVLNVNSNKAVEVANASTSNGALIQQNWYRGDLNQQWQLVKIN

SEQ ID NO: 41 - protein, 537 aa, Paenibacillus chitinolyticus
WVGMPMGKLHVNGKNLVNSNNQPVLLNGWHQPSGAYWTYQDSNYYLNLHGNNRHAATLAYLKDITDTFADTSPKYGSNHGWNMNQVRLFIDRQDMGDVAAGTYNFAGVQTVTQNVIIPYIQYAKSKGVYVVLGLDFTLKDDQATTPANLQKFNEIWGYLASRPEIKSADNVHFELINEPVKSYANGHWGGYNGENDFVDHWNDLRNFQNSMISTIRSKGADNVIWAAGLGYNQFYSLTASHPLTDPLNNYGYAVHWYPGYGAYDNFSILQDQWNTNVKAAADKYPINITEVTWFKNKPGDSAYWNLFNGSNEGFGTNTKTIFNAAGNVSIAAHMNGFILSEGPRSSFADPTAGLKWDGDASRSAMGRFLFNWYHERAQTYPGGGQGGPTTGLVSGATYKIVARHSNKVIDVPGGQNENNLQLQQWSDLGGNPQKWVLTSIGSGNYTLTSVNSPDKVIDIRNGTLTNGEAVQLMSNLNTTAQHFKVNDLGNGYWSIINVNSNKAIEVANASTSDGAKLQQNTYTGATNQQWKFVAVSN

SEQ ID NO: 42 - protein, 391 aa, Bacillus sp-18423
AASDVTVNVSAEKQVIRGFGGMNWPAWAGDLTAAQRETAFGNGQNQLGFSILRIHVDENRNNWYKEVETAKSALKLGAIVFASPWNPPSDMVETFNRNGDTSAKRLKYNKYAAYAQHLNDFVTFMKNNGVNLYAISVQNEPDYAHEWTWWTPQEMLRFMRENAGSINARVIAPESFQYLKNLSDPILNDPQALANMDILGTHLYGTQLSQFPYPLFKQKGAGKDLWMTEVYYPNSDTNSADRWPEALDVSQHIHNAMVEGDFQAYVWWYIRRSYGPMKEDGTISKRGYNMAHFSKFVRPGYVRIDATKNPNANVYVSAYKGDNKVVIVAINKSNTGVNQNFVLQNGSASNVSRWITSSSSNLQPGTNLTVSGNHFWAHLPAQSVTTFVVNR

SEQ ID NO: 43 - protein, 746 aa, Aspergillus fumigatus
LNTFPDCSSGPLSKLAVCDTSLDVTTRAQSLVNAMTFEEKVNNTQYNSPGVPRLGLPAYNWWSEALHGVAGSPGVEFADSGPFSYATSFPQPILLGATFDDDLIKQVATVVSTEGRAFGNAGRSGLDFWTPNINPFRDARWGRGQETPGEDPLHVSRYVYHLVDGLQNGIGPANPKVVATCKHFAAYDLEDWNGVVRHSFNAEVSTQDLSEFYLPPFKSCARDARVDAVMCSYNALNGVPACADSYLLQTILREHWKWDEPGRWITSDCGAIDDIYNGHNFTTTPAEAAATALNAGTDLDCGTVFPKYLGQAADEGLYSNQTLDRALVRLYSSLVKLGYFDPAEDQPYRSIGWTDVDTPAAEALAHKAAGEGIVLLKNDKTLPLKAKGTLALIGPYANATKQMQGNYEGPAKYIRTLLWAATQAGYDVKYAAGTAINTNSTAGFDAALSAAKQADVVVYAGGIDNTIEAEGRDRTTIAWPGNQVNLIDQLSKIGKPLVVVQFGGGQVDDSSLLSNPRVNALLWAGYPSQEGGSAIFDILTGKTAPAGRLPVTQYPADYVNQVPMTDMALRPGSNTPGRTYRWYDKAVLPFGFGLHYTTFKISWPRRALGPYNTAALVSRSPKNVPIDRAAFDTFHIQVTNTGKTTSDYVALLFLKTTDAGPKPYPLKTLVGYTRAKQIKPGEKRSVDIEVSLGSLARTAENGDLVLYPGRYTLEVDVGESQYPTASFTVTGKETILDSFPQPPKTR

SEQ ID NO: 44 - protein, 785 aa, Aspergillus nidulans
QANTSYTDYNVEANPDLFPLCLQHLNASFPDCASGPLSLTPVCDRSLSPKDRATALVSLFTFDELVNNTGNTGLGVSRLGLPNYQVWGEALHGVGRANFVESGNFSWATSFPMPITMMAALNKTLIHQIGTIVSTQLRAFSNAGLGGVDVYSPNINTFRHPVWGRGQETPGEDAFLTSVYGYEYITALQGGVDPETLKIIATAKHYAGYDIESWNNHSRLGNDMQITQQELSEYYTPPFIVASRDAKVRSVMCSYNAVNGVPSCANKFFLQTLLRDTFEFSEDGYVSGDCGAVYNVWNPHGYASNEAAASADSILAGTDIDCGTSYQWHSEDAFEDSLVSRSDIERGVIRLYSNLVQAGYFDGEDAPYRDITWDDVLSTDAWNIAYEAAVEGIVLLKNDETLPLSKDIKSVAVIGPWANVTEELQGNYFGPAPYLISPLTGFRDSGLDVHYALGTNLTSHSTSGFEEALTAAKQADAIIFAGGIDNTIEAEAMDRENITWPGNQLDLISKLSELGKPLVVLQMGGGQVDSSSLKDNDNVNALIWGGYPGQSGGHALADIITGKRAPAGRLVTTQYPAEYAEVFPAIDMNLRPNETSGNPGQTYMWYTGTPVYEFGHGLFYTTFEESTETTDAGSFNIQTVLTTPHSGYEHAQQKTLLNFTATVKNTGERESDYTALVYVNTTAGPAPYPKKWVVGFDRLGGLEPGDSQTLTVPVTVESVARTDEQGNRVLYPGSYELALNNERSVVVKFELKGEEAVILSWPEDTTSDFVSSIDGGLDRKQDVIA

SEQ ID NO: 45 - protein, 775 aa, Talaromyces emersonii
QDNQTYANYSSQSQPDLFPRTVATIDLSFPDCENGPLSTNLVCNKSADPWARAEALISLFTLEELINNTQNTAPGVPRLGLPQYQVWNEALHGLDRANFSHSGEYSWATSFPMPILSMASFNRTLINQIASIIATQARAFNNAGRYGLDSYAPNINGFRSPLWGRGQETPGEDAFFLSSTYAYEYITGLQGGVDPEHVKIVATAKHFAGYDLENWGNVSRLGFNAIITQQDLSEYYTPQFLASARYAKTRSIMCSYNAVNGVPSCANSFFLQTLLRENFDFVDDGYVSSDCDAVYNVFNPHGYALNQSGAAADSLLAGTDIDCGQTLPWHLNESFVEGYVSRGDIEKSLTRLYSNLVRLGYFDGNNSEYRNLNWNDVVTTDAWNISYEAAVEGITLLKNDGTLPLSKKVRSIALIGPWANATVQMQGNYYGTPPYLISPLEAAKASGFTVNYAFGTNISTDSTQWFAEAIAAAKKSDVIIYAGGIDNTIEAEGQDRTDLKWPGNQLDLIEQLSQVGKPLVVLQMGGGQVDSSSLKANKNVNALVWGGYPGQSGGAALFDILTGKRAPAGRLVSTQYPAEYATQFPANDMNLRPNGSNPGQTYIWYTGTPVYEFGHGLFYTEFQESAAAGTNKTSTFDILDLFSTPHPGYEYIEQVPFINVTVDVKNVGHTPSPYTGLLFANTTAGPKPYPNKWLVGFDWLPTIQPGETAKLTIPVPLGAIAWADENGNKVVFPGNYELALNNERSVVVSFTLTGDAATLEKWPLWEQAVPGVLQQ

SEQ ID NO: 46 - protein, 659 aa, Herbinix hemicellulosilytica
MAKITILEDKIIFERRDELTVIEAYGANCLRYRSTRNIKLSDENWTLLPPTTKADCKTFGNEREATIVNGDISATIRDGFGWTGASISYFRKGKQILRSRDEGDPVTRFTHLGGNNYQVKAIFDSNPGEHIYGLGQEQEDQFDRKGCTCNIMHYNTKSTLPVIYSSLGYGFLWNNPAPGRCETTFNHTMWVADSAYQIDYLVYVGDTPADIMKIYCDLTGYAPKFPDWAAGFWQSKLRYESQEDLLEVAREYKKRGIPLAAIVIDYFHWTEQGEWKFDPNLWPDPKAMCDELESMGVKPVVSIWPTINPKSENYHAMDNENMLVRTENGQYGIFNFYGLQTFIDPTNPKTGPFVWSKVRENYYKYGIKNFWLDEAEPEVHPQQFGNLRFYIGNGAQTALLYPYYYAKIFYDGLRSEGEEDIIILTRAAYPGSQRFGSLVWNGDIPSTFKALRQSVTSGLSMSMCGIPWWNSDIGGFHSADIESDYFRELIVRWFQFGLFSPVMRLHGARNRTKNQVDRHPGVKERSGGDNEIWSFGERNYPILKNIIELRERLKPYILKYMDIASKTGSPIMRPMFYDFYDDEICYRLDDQYMFGEDILFAPILEQGVTQRSVYLPEGKWVNVNDKKTYEGRQMITARAELHEFIAFVRSGSEVLEVFS

SEQ ID NO: 47 - protein, 407 aa, Bacillus sp. KK-1
AVHSKTPDILGTTGKNNLNQAYKKYFDTKGDGKGGSLFHYMKDGSAYIASTTDDNLLGNGYYSVKTEGMSYGMMITLQMNDEYKFQKLWDFVRKYMRHSDRNDSLYGYHSWHMKTNGSDVQTIDQNVASDGEVWFAAALMMASGRWGDKKYPYDYKARAQDMLDALAGDGEYANTGKESRVFIKNSKDQRYAMVRFGPYVNWTDPSYHVPAFFELFAKSAKSSQQYFWKDAANKSRTYLSETTFKSVLNNGSTVTNAATGLFPDEAGFDGVSDAAHSSTETDRNFSYDAWRTVSHIAMDHTLWSSADNAYRASEQKAVNKFLTFMKRENYGRTAHEYTLNGTAVKKGSPVGLIAANAGGATAASDASLRTGFANAFNSTYIPEDYYGSCLYMLNSLVANGKFAMYLP

SEQ ID NO: 48 - protein, 384 aa, Aspergillus aculeatus
VGLDQAAVAKGLQYFGTATDNPELTDIPYVTQLNNTADFGQITPGNSMKWDATEPSQGTFTFTKGDVIADLAEGNGQYLRCHTLVWYNQLPSWVTSGTWTNATLTAALKNHITNVVSHYKGKCLHWDVVNEALNDDGTYRTNIFYTTIGEAYIPIAFAAAAAADPDAKLFYNDYNLEYGGAKAASARAIVQLVKNAGAKIDGVGLQAHFSVGTVPSTSSLVSVLQSFTALGVEVAYTEADVRILLPTTATTLAQQSSDFQALVQSCVQTTGCVGFTIWDWTDKYSWVPSTFSGYGAALPWDENLVKKPAYNGLLAGMGVTVTTTTTTTTATATGKTTTTTTGATSTGTTAAHWGQCGGLNWSGPTACATGYTCTYVNDYYSQCL

SEQ ID NO: 49 - protein, 206 aa, Thermomyces lanuginosus
FPAGNATELEKRQTTPNSEGWHDGYYYSWWSDGGAQATYTNLEGGTYEISWGDGGNLVGGKGWNPGLNARAIHFEGVYQPNGNSYLAVYGWTRNPLVAYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVG

SEQ ID NO: 50 - protein, 491 aa, Bacillus stearothermophilus
AAPFNGTMMQYFEWYLPDDGTLWTKVANEANNLSSLGITALWLPPAYKGTSRSDVGYGAYDLYDLGEFNQKGTVRTKYGTKAQYLQAIRAAHAAGMQVYADVVFDHKGGADGTEWVDAVEVNPSDRNQVISGTYQIQAWTKFDFPGRGNTYSSFKWRWYHFDGVDWDESRKLSRIYLFEGKAWDWEVDTEFGNYDYLMYADLDMDHPEVVTELKNWGKWYVNTTNIDGFRLDAVKHIKFSFFPDWLSYVRSSTGKPLFTVGEYWSYDINKLHNYITKTDGTVSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARP

SEQ ID NO: 51 - protein, 491 aa, Bacillus stearothermophilus
AAPFNGTMMQYFEWYLPDDGTLWTKVANEANNLSSLGITALWLPPAYKGTSRSDVGYGAYDLYDLGEFNQKGTVRTKYGTKAQYLQAIQAAHAAGMQVYADVVFDHKGGADGTEWVDAVEVNPSDRNQVISGTYQIQAWTKFDFPGRGNTYSSFKWRWYHFDGVDWDESRKLSRIYLFSGKQWDWPVDNEFGNYDYLMYADLDMDHPEVTTELKNWGKWYVNTTNIDGFRLDAVKHIKFYFFPDWLSYVRSSTGKPLFTVGEYWSGDINKLHNYITITDGTVSLFDAPLHYKFYNASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARP

SEQ ID NO: 52 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYGFEKVGYYNPTDGTWTIKVVSYSGSANYQVDVVSDGSLSQPGSS

SEQ ID NO: 53 - protein, 328 aa, Thermotoga maritima
MQNVSLRELAEKLNIYIGFAAINNFWSLSDAEKYMEVARREFNILTPENQMKWDTIHPERDRYNFTPAEKHVEFAEENDMIVHGHTLVWHNQLPGWITGREWTKEELLNVLEDHIKTVVSHFKGRVKIWDVVNEAVSDSGTYRESVWYKTIGPEYIEKAFRWAKEADPDAILIYNDYSIEEINAKSNFVYNMIKELKEKGVPVDGIGFQMHIDYRGLNYDSFRRNLERFAKLGLQIYITEMDVRIPLSGSEEYYLKKQAEVCAKIFDICLDNPAVKAIQFWGFTDKYSWVPGFFKGYGKALLFDENYNPKPCYYAIKEVLEKKIEERK

SEQ ID NO: 54 - protein, 556 aa, Gloeophyllum sepiarium
QSVDSYVSSEGPIAKAGMLANIGPNGSKASGASAGVVVASPSKSDPDYWYTWTRDSSLVFKSLIDQYTTGIDSTSSLRTLIDDFVTAEANLQQVPNPSGTLTTGGLGEPKFNVDETAFTGPWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYYGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTKIEYKYIRKMNGAVTWESDPNNSITTPASGSWTENDTWR

SEQ ID NO: 55 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFDDASLYHPKCTIDYNNQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 56 - protein, 1020 aa, Talaromyces funiculosus
LPFNERVDQVLRSYEVTSKLDSRSTKPSKHGHTYQTQFLGVTWDQRNWRLQSTVLDQGHYESRGSIANGYIGLNVAGAGPLFELDSPVDGDVINGWPLFSRRQTFAGLAGFYDLQPRTNGTNFPWLSQYGDDSAISGVPHWGGMVLDLGDGEYLDATVDNSTISDYTTTYDYKAGVLSWDYKWTPKNANGSFGISYKIFANKLDVNQAVVQLSITPSTNGSASVVNVIDGYAAVRTDFVSSGNESDVVYTAVKPNGVTNVTAWIYTALDGDDAFDISSAALVNDKPYVHQNDSSIAQSVNVTFTAGTTITINKFVGAASTDAFPDPQSTAREAALSARRRGFDDLFRSHISEWAQVMPDDSVDDFTLANGTLPNDTFIIESAVMAVVNPYYLLQNTVGPNALRRVNNAPVNDWSIPVGGLTSDSYAGQIFWDADVWMQPGLVAAFPESAKRITNYRAAKYSQALENAKTAYTSSQNQTWFSPDAAIYSWTSGRVGNCTATGPCWDYEYHLNGDIGISLVNEWVVSGDNETFKNKHFPIYNSIATLYGDLLKKNGSYYTLTNMTDPDEYANNVDAGGYTMTLISQTLSNANAFRKQFGMNENTTWTEMADNILLIRENDVTLEYTTMNNSVAVKQADVILSTFPLDYTKNYTTSAALNDLDYYALKQSPDGPGMTYAIFSIVANDVSPSGCSAYTYAQYSYDPYIRGPFFQFSEQLLDDYTINGGTHPAFPFLTGHGGANQVVLYGYLGLRLLPDDMLHIDPNLPPQIPSIKYRTFYWRGWPIQAASNYTHTTIQRATTVAPLSTADPTYANKSIHVSVGHNTVNSTTYSLSANGSALVVPNRQIGSINTVAGNVVQCKSVLSTDAYQKGQYPISAVDGAASTKWQPEFAANISSLTVDLTGSNVSSVSGFYFDWAQAPPTNITVLLHNSSSAALASSGDKPGSSAVTLNITISNPYNASTYNANIIALPSSNSTNYTFPAPVPKPRYATLFVQGNQALDETDTKSGNGTGATVAEWAILS

SEQ ID NO: 57 - protein, 844 aa, Aspergillus fumigatus
QELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 58 - protein, 506 aa, Aspergillus fumigatus
QQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 59 - protein, 437 aa, Trichoderma reesei
QQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL